(19) United States
(12) Patent Application Publication
(10) Pub. No.: US 2026/0297622 A1
(43) Pub. Date: Oct. 1, 2026

(54) CELL

(71) Applicant: ANTION BIOSCIENCES SA, Plan-les-Quates (CH)

(72) Inventors: Marco ALESSANDRINI, Plan-les-Quates (CH); Antonija ŠAKIC, Plan-les-Quates (CH); Audrey ROUSSEL-GERVAIS, Plan-les-Quates (CH); Inès LANZ, Plan-les-Quates (CH); Sten ILMJÄRV, Plan-les-Quates (CH)

(73) Assignee: ANTION BIOSCIENCES SA, Plan-les-Quates (CH)

(21) Appl. No.: 19/481,129

(22) PCT Filed: May 3, 2024
(86) PCT No.: PCT/EP2024/062349

§ 371 Date: Nov. 3, 2025

(30) Foreign Application Priority Data
May 4, 2023 (GB) 2306619.4

(51) Int. Cl. C12N 15/861; A61K 31/711; A61K 35/12; A61K 35/28; A61K 35/30; A61K 35/39; A61K 35/545; A61P 35/00; C07K 14/705; C12N 5/071; C12N 5/0735; C12N 5/074; C12N 5/077; C12N 5/0775; C12N 5/0783; C12N 5/0789; C12N 5/0797; C12N 15/864; C12N 15/867

(52) U.S. Cl. CPC C12N 15/861; A61K 31/711; A61K 35/28; A61K 35/30; A61K 35/39; A61K 35/545; A61P 35/00; C07K 14/7051; C07K 14/70539; C12N 5/0606; C12N 5/0623; C12N 5/0636; C12N 5/0647; C12N 5/0659; C12N 5/0662; C12N 5/0676; C12N 5/0696; C12N 15/8645; C12N 15/867; A61K 2035/124; C12N 2310/141; C12N 2310/531; C12N 2510/00; C12N 2800/10; C12N 2830/002; C12N 2830/008; C12N 2830/34; C12N 2840/203

(57) ABSTRACT

The present invention relates to donor cells that are engineered for use in universal donor cell therapies, for allogeneic, off-the-shelf administration, and that possess improved persistence in the immune system of a host. The present invention also relates to the use of miRNA expression constructs in such engineered donor cells.

### BACKGROUND OF THE INVENTION

### Field of the Invention

The present invention relates generally to the field of cell biology. More particularly, it concerns the use of miRNA gene constructs to create universal donor cell (UDC) therapies for allogeneic, off-the-shelf administration, particularly where cells can persist for longer by avoiding rejection by a patient's immune system.

### Description of Related Art

Autologous cell therapies, such as chimeric antigen receptor (CAR) T-cell therapy, have been established as an effective treatment for conditions such as haematological malignancy. Such cell-therapy based approaches are however challenged by high manufacturing costs and complicated logistics, which limit widespread adoption. The ability to use allogeneic cells, i.e. cells that are genetically dissimilar to the patient, would be a promising solution to overcome these challenges, since they would allow for manufacturing of numerous therapeutic doses from a single healthy donor unit, significant cost reductions and off-the-shelf provision.

However, to fully utilise allogeneic cell-based strategies, it will be necessary to avoid immune rejection and enhance the capability for allogenic cell persistence in the context of the immune system of the host/patient. This will be needed in order to obtain a more durable clinical response. Thus, there is a need for strategies to persistently, e.g. permanently, modify donor cells to provide engineered allogeneic cells with improved persistence in vivo.

The major histocompatibility complex (MHC) is a term used to describe a group of genes in animals and humans that encode a variety of cell surface markers, antigen-presenting molecules, and other proteins involved in immune function. The human leukocyte antigen (HLA) complex is synonymous with the human MHC (Viatte S, Scur PH, Seo P. Human leukocyte antigens (HLA): A roadmap. In: UpToDate, Post TW (Ed), UpToDate, Waltham, MA.).

HLA/MHC class I deficiency, also referred to as Bare Lymphocyte Syndrome I (BLS I), is a form of severe combined immunodeficiency syndrome (SCID) characterized by defects in the expression of molecules involved in cell surface expression of HLA-I molecules. Most commonly BLS I is accounted to genetic mutations in TAP proteins, namely TAP1, TAP2 and TAPBP (Online Mendelian Inheritance in Man (OMIM), 604571). Cases of beta2-microglobulin (B2M) deficiency have also been reported, with patients presenting with similar features of typical HLA I deficiency, but with more extensive immunological defects (Ardeniz et al., 2015, PMID: 25702838).

HLA/MHC class II deficiency, also referred to as Bare Lymphocyte Syndrome I (BLS II), is another form of SCID characterized by defects in the expression of molecules involved in cell surface expression of HLA-II molecules. Manifestation of BLS II is most commonly due to genetic defects in transcription factors involved in the expression of HLA-II, namely CIITA, RFXANK, RFX5 and RFXAP (Online Mendelian Inheritance in Man (OMIM), 209920).

Allogenic infusion of engineered donor cells, such as CAR T-cells, will further necessitate abrogation of endogenous T cell receptor (TCR) expression to mitigate alloreactivity of donor-derived T-cells and graft vs host disease (GvHD). This can be achieved by various gene engineering approaches, most typically using gene editing techniques such as CRISPR knockout. However, there is still a need for further alternative and improved approaches to provide allogeneic TCR-deficient T-cells for this purpose.

In short, the present invention addresses each of these problems and addresses the need in the art to provide improved and effective allogeneic engineered donor cells.

### SUMMARY OF THE INVENTION

The present invention is defined in the accompanying claims. Statements in the description are to illustrate and further aid the understanding of the invention as claimed.

In a first aspect, the present invention provides an engineered donor cell with reduced rejection by the immune system of a host, wherein one or more cell surface-expressed polypeptides involved in immune signalling are functionally modulated.

In a second aspect, the present invention provides a miRNA expression construct comprising one or more miRNA hairpins targeting B2M, NLRC5, TAP1, TAP2, TAPBP, RFX5, RFXANK, RFXAP, CIITA, TCRa, TCRb, CD3d, CD3g, CD3e and/or CD3z. In an embodiment, the construct further comprises an expressed transcript.

In a third aspect, the present invention provides a DNA molecule comprising the miRNA expression construct of the invention.

In a fourth aspect, the present invention provides a plasmid comprising the miRNA expression construct or DNA molecule of the invention.

In a fifth aspect, the present invention provides a vector comprising the miRNA expression construct, DNA molecule or plasmid of the invention.

In a sixth aspect, the present invention provides an engineered donor cell comprising the miRNA expression construct, DNA molecule, plasmid or vector of the invention.

In an seventh aspect, the present invention provides a method for down-regulating a polypeptide in a cell comprising expressing the miRNA expression construct, DNA molecule, plasmid or vector of the invention in the cell.

In an eighth aspect, the present invention provides a method for preparing an engineered donor cell comprising transfecting or transducing a cell with the miRNA expression construct, DNA molecule, plasmid or vector of the invention.

In a ninth aspect, the present invention provides a method for preparing an engineered donor cell from a patient donor or healthy donor comprising:
(a) collecting a cell from the patient; and
(b) transfecting or transducing the cell with the miRNA expression construct, DNA molecule, plasmid or vector of the invention; and
(c) expressing the miRNA expression construct.

In a tenth aspect, the present invention provides an engineered effector cell obtainable or obtained by the method of the invention.

In an eleventh aspect, the present invention provides a composition comprising the engineered donor cell of the invention.

In a twelfth aspect, the present invention provides the engineered donor cell, miRNA expression construct, DNA molecule, plasmid, vector or composition of the invention, for use in therapy.

In a thirteenth aspect, the present invention provides the engineered donor cell, miRNA expression construct, DNA molecule, plasmid, vector or composition of the invention, for use in a method of treating cancer, an infectious disease, an auto-immune disease or an inherited disorder.

In a fourteenth aspect, the present invention provides a method of treating cancer, an infectious disease, an auto-immune disease or an inherited disorder, comprising administering the engineered donor cell, miRNA expression construct, DNA molecule, plasmid, vector or composition of the invention.

In a fifteenth aspect, the present invention provides the engineered donor cell, miRNA expression construct, DNA molecule, plasmid, vector or composition of the invention, for use in the manufacture a medicament for the treatment of cancer, an infectious disease, an auto-immune disease or an inherited disorder.

In a sixteenth aspect, the present invention provides the engineered donor cell, miRNA expression construct, DNA molecule, plasmid, vector or composition of the invention, for use in a method of stem cell therapy.

In a seventeenth aspect, the present invention provides a method of stem cell therapy, comprising administering the engineered donor cell, miRNA expression construct, DNA molecule, plasmid, vector or composition of the invention.

In a eighteenth aspect, the present invention provides the engineered donor cell, miRNA expression construct, DNA molecule, plasmid, vector or composition of the invention, for use in the manufacture a medicament for stem cell therapy.

### BRIEF DESCRIPTION OF THE DRAWINGS

FIG. 1: Downregulation of HLA-ABC expression in gene-modified primary T-cells using target sequences for B2M miRNAs, presented as normalized values of HLA-ABC median fluorescence intensity (MFI), relative to the expression derived from T-cell modified with a scrambled control miRNA.

FIG. 2: Gene-engineered T-cells with varied HLA class I expression. Flow cytometric histograms of HLA-ABC expression and accompanying descriptive statistics. The Comp-FL7 channel reflects the HLA-ABC median fluorescence intensity (MFI, right-hand column of table). Percentages on the left of the histograms indicate normalized HLA-ABC expression, relative to control-transduced T-cells called Scrambled-mCherry (mCherry only).

FIG. 3: Downregulation of HLA class I expression. Flow cytometric histograms and normalized values for the HLA-ABC expression. Histograms are based on gating on gene modified cells, i.e., those positive for mCherry reporter gene.

FIG. 4: Flow cytometry gating strategy using B2M_T5 as an example. Gating was first based on T-cell selection, doublet and dead cell exclusion. Thereafter, mCherry vs HLA-ABC was plotted into overlayed histograms to assess silencing of HLA-ABC expression in gene-modified (mCherry positive) and unmodified cells.

FIG. 5: Multi-hairpin miRNA constructs against HLA class I using B2M_T5. Histograms demonstrate partial improvements in HLA-ABC silencing when increasing B2M_T5 to two and three hairpin (hp) miRNA constructs. Normalized expression levels reflect >90% silencing of HLA class I (n=3 donors).

FIG. 6: Gene engineering HEK293 cells with varied HLA class I expression. Flow cytometric histograms of HLA-ABC expression and accompanying descriptive statistics. The YL2 channel reflects mCherry reporter gene expression, while HLA-ABC median values are listed under VL1 channel (final column in the table). Percentages on the left of the histograms indicate normalized HLA-ABC gene silencing, relative to control-transduced HEK293 cells (mCherry only).

FIG. 7: Downregulation of TCR a/b expression. Flow cytometric histograms and normalized values for the percentage cells expressing TCR a/b and MFI expression. Histograms are based on gating on gene modified cells, i.e., those positive for mCherry reporter gene.

FIG. 8: Flow cytometry gating strategy using TRAC_T1 as an example. Gating was first based on T-cell selection, doublet exclusion and gating on viable cells. Thereafter, mCherry vs TCR a/b and CD3e were plotted to assess silencing of TCR expression in gene-modified (mCherry positive) and unmodified cells.

FIG. 9: Downregulation of TCR a/b expression. Flow cytometric histograms and normalized values for the percentage of cells expressing TCR a/b and MFI levels. Histograms are based on gating on gene modified cells, i.e., those positive for mCherry reporter gene.

FIG. 10: Flow cytometry gating strategy using CD3z_T1 as an example. Gating was first based on T-cell selection, doublet exclusion and gating on viable cells. Thereafter, we plotted mCherry vs TCR a/b and CD3e to assess silencing of TCR expression in gene-modified (mCherry positive) and unmodified cells.

FIG. 11: Efficiency of TCR silencing in Jurkat cells. Single hairpin CD3z_T2 constructs resulted in complete silencing of TCR a/b expression (unstained controls in black). Similarly, >90% reduction on CD3e on the surface of Jurkat cells was shown.

FIG. 12: Multi-hairpin miRNA constructs against TCR using CD3z_T2. Histograms demonstrate minor improvements in TCR silencing when increasing CD3z_T2 to two and three hairpin (hp) constructs. Normalized expression levels reflect >95% silencing of the TCR.

FIG. 13: Histograms of multi-targeting constructs to silence TCR expression. Except untransduced (UTD) condition, all histograms reflect cell surface expression in gene-modified T-cells (gated on mCherry positive cells). “Freq. of Parent” column indicates the percentage of cells gated for creation of the histograms, i.e. the percentage of transduced T-cells. TCR a/b reported in the BL1 channel, CD3e in RL2. *=Higher copy number transduction (MOI=2.0).

FIG. 14: Mixed lymphocyte reaction (MLR) using CD3z_T2 and TRAC_T1 modified T-cells. (A) PBMCs from the same donor were transduced with a miRNA against either CD3z_T2 or TRAC_T1 target sequences. With transduction rates of 65-80%, T-cells with 95% and 30% TCR silencing were created. Control transduced T-cells carried only the mCherry reporter gene and expressed TCR levels equivalent to untransduced T-cells (UTD). (B) CD137 expression in unstimulated, MLR (1:1 ratio with irradiated, unmatched PBMCs), and CD3/CD28 microbead activated cells (positive controls). No CD137 expression was observed in all unstimulated PBMC populations. In the MLR, a notable decrease in CD137 expression was observed in CD8+ T-cells transduced with our TRAC_T1 construct (see upper quadrants relative to the lower unmodified cells in the lower quadrants). With CD3z_T2 transduced cells, a significant loss of CD137 expression was observed (lower panel, upper quadrants). When normalized to the CD137 expression in control (mCherry only) transduced CD8+ T-cells, there was a less than 5% activation of cells transduced with CD3z_T2 constructs. Even in the CD3/CD28 microbead activated condition, a remarkable decrease in CD137 expression when using the CD3z_T2 construct was observed.

FIG. 15: Screening miRNAs for silencing of HLA class II expression. A. Summary of transduction rates and silencing of HLA-II expression in primary T-cells, demonstrating equivalent gene modification rates and construct pATN504 achieving most efficient silencing of HLA-CP/DQ/DR cell surface expression. B. Flow cytometric histograms of primary T-cells modified with CIITA targeting miRNA constructs. (2nd screen). Histograms are based on gating on gene modified cells, i.e., those positive for mCherry reporter gene.

FIG. 16: Engineering of allogeneic and hypoimmunogenic CAR19 T-cells with tuned silencing of HLA-I expression. All bimodal gene constructs were designed to silence cell surface expression of the TCR via the use of optimized miRNAs targeting CD3z, and to co-express an anti-CD19 CAR (CAR19) the RQR8 reporter gene. Constructs pATN292 and pATN293 were designed to additionally silence HLA class I expression using miRNA targeting B2M_T2 and B2M_T5, respectively. Finally, pATN294 expresses dual miRNAs targeting B2M_T5 to achieve maximal silencing of HLA-I.

FIG. 17: Production and immunophenotypic characterization of allogeneic and hypoimmunogenic miCAR19 T-cells. A. Schematic representation of miCAR T-cell production process. B. Flow cytometry dot plots of miCAR 19 T-cells (pATN293, with silencing of TCR α/β and HLA-ABC) before and after depletion of TCR α/β positive cells. CAR positive cells were detected based on CD34 positivity (RQR8). Remaining cells after TCR α/β depletion are devoid of TCR α/β, silenced for HLA-ABC, and fully express RQR8 reporter gene (CD34). C. Flow cytometry histograms representing cell surface expression of HLA-ABC in engineered miCAR19 T-cells. Cells modified with pATN296 express similar levels of HLA-ABC as untransduced T-cells. Cells modified with constructs carrying B2M_T2 and B2M_T5 targeting miRNA (pATN292 and pATN293) have downregulated expression of HLA-ABC (80 and 90%, respectively), while cells modified with construct carrying dual-miRNAs of B2M_T5 have most efficient downregulation HLA-ABC (95%).

FIG. 18: Extended immunophenotypic characterization of allogeneic and hypoimmunogenic miCAR19 T-cells. All cell products were analyzed using flow cytometry at 24 hours post thawing. For positive controls, untransduced and CAR19 T-cells were activated with CD3/CD28 microbeads on the day of thawing. UTD: Untransduced T-Cells UTD act: Untransduced T-Cells; CAR19: CAR19 T-cells activated with CD3/CD28 microbeads; CAR19 act: CAR19 T-cells activated with CD3/CD28 microbeads; 296: CAR19 T-cells with TCR silencing; 294: CAR19 T-cells with TCR and HLA-I silencing (B2M_T5_T5), 5% remaining HLA-I; 293: CAR19 T-cells with TCR and HLA-I silencing (B2M_T5), 10% remaining HLA-I; 292: CAR19 T-cells with TCR and HLA-I silencing (B2M_T2), 20% remaining HLA-I. A. Ratio of CD4+/CD8+ T-cells indicates no differences across all manufactured T-cell products. B. PD1, TIGIT and TIM3 expression levels were shown to be lowly expressed in both CD4 and CD8 T-cell populations after manufacturing. Positive controls (CD3/CD28 microbead activated) highly expressed PD1, TIGIT and TIM3. C. Memory phenotypes, based on CD45RA and CD62L expression, indicate that CD4+ T-cells were mostly central memory (TCM), while CD8+ T-cells were predominantly of naïve/stem cell memory (TSCM) phenotype. Minor proportions of effector memory (TEM) and effector memory T-cells re-expressing CD45RA (TEMRA) were present, except in activated T-cell populations. D. Activation status, as measured by the presence of CD69 and CD25 positive cells, indicates negligible activation of all manufactured T-cell products.

FIG. 19: Specific cytotoxicity of tumor cells by allogeneic and hypoimmunogenic miCAR 19 T-cells. UTD: Untransduced T-Cells; 296: CAR 19 with TCR silencing; 294: CAR 19 with TCR and HLA-I silencing (B2M_T5_T5), 5% remaining HLA-I; 293: CAR 19 with TCR and HLA-I silencing (B2M_T5), 10% remaining HLA-I; 292: CAR 19 with TCR and HLA-I silencing (B2M_T2), 20% remaining HLA-I. A. In short-term cytotoxicity assays, miCAR19 T-cells with varied silencing of HLA-I perform equally well in terms of functional activity against CD19-expressing tumor cells. Engineered miCAR19 T-cells (effector, E) and JeKo-1 cells (target, T) were co-cultured at E:T ratios of 1:1 and 3:1 over 24, 48 and 72 hours. Target cell survival (JeKo-1 cells expressing GFP) was assessed by detection of GFP positivity on flow cytometry after the indicated time points. Data point on the graphs represent the mean and standard deviation from n=3 donors. Dotted line on each representative graph indicates the % of JeKo-1 cells initially plated in co-culture. B & C show that allogeneic and hypoimmunogenic miCAR19 T-cells maintain efficient cytotoxicity of CD19-expressing tumor cells. B. In short-term cytotoxicity assays, miCAR19 T-cells with varied silencing of HLA-I perform equally well in terms of functional activity against CD19-expressing tumor cells. Engineered miCAR 19 T-cells (effector, E) and JeKo-1 cells (target, T) were co-cultured at E: T ratios of 1:9, 1:3, 1:1 and 3:1. Target cell survival (JeKo-1 cells expressing GFP) was assessed by detection of GFP positivity on flow cytometry after the indicated time points. Data point on the graphs represent the mean and standard deviation from n=3 donors. C. In a long-term assay, miCAR19 T-cells with 80% silencing of HLA-I (engineered from construct 292) proved to efficiently deplete JeKo-1 tumor cells over four rounds of re-stimulation with the same number of target cells. D. Allogeneic and hypoimmunogenic miCAR19 T-cells maintain efficient cytotoxicity of CD19-expressing tumor cells. In recursive killing assays, all miCAR19 T-cells with varied silencing of HLA-I perform equally well in terms of lysing target cells over four rounds of JeKo-1 tumor cell exposure, with tumor cells added to the culture at 1, 3, 7 and 10 days, while untransduced T-cells were unable to control tumor growth.

FIG. 20: Mixed lymphocyte reactions with unmatched CD8+ T-cells and NK cells using allogeneic and hypoimmunogenic miCAR19 T-cells. UTD: Untransduced T-Cells, 296: CAR19 with TCR silencing; 294: CAR 19 with TCR and HLA-I silencing (B2M_T5_T5), 5% remaining HLA-I; 293: CAR19 with TCR and HLA-I silencing (B2M_T5), 10% remaining HLA-I; 292: CAR19 with TCR and HLA-I silencing (B2M_T2), 20% remaining HLA-I. A. Host PBMCs were primed with mitomycin treated graft donor cells (CAR19 T-cells), after which CD8 positive T-cells were isolated and labelled with CellTrace Violet (CTV) dye. Primed CD8+ T-cells (effector, E) were co-cultured with graft miCAR19 T-cells (target cells, T) at an E: T ratio of 1:1. Six days post plating the co-culture, cells were analyzed by means of flow cytometry. While untransduced (UTD) and allogeneic miCAR 19 T-cells (296, with no silencing of HLA-I) were mostly depleted by primed CD8+ T-cells, all miCAR19 T-cell populations with HLA-I silencing (292, 293, and 294) remained equally protected, irrespective of whether they had 80%, 90% or 95% HLA-I silencing. B. Primed CD8+ T-cells were activated and proliferated only in co-culture with UTD and 296 miCAR T-cells, as illustrated by the dilution of CTV signal. Corresponding with the lack of cytotoxicity of HLA-I silence miCAR T-cells, an equal signal of CTV was observed for primed CD8+ T-cells exposed to the same miCAR19 T-cell populations. C. Host NK cells (effector cells, E) were co-cultured with graft miCAR19 T-cells (target cells, T) at an E:T ratio of 5:1. After 24 hours, cells were analyzed by means of flow cytometry to assess for the proportions of NK cells and T-cells based on CD56 and CD5 expression, respectively. Graft UTD and miCAR19 T-cells with full expression of HLA-I (pATN296) remained equally protected, while miCAR19 T-cells with highest silencing of HLA-I (294) were the most sensitive to NK cell mediated cytotoxicity. Graft miCAR 19 T-cells with 80% (292) and 90% (293) HLA-I silencing were correspondingly protected from NK cell mediated cytotoxicity. C & D show that tuned silencing of HLA-I protects miCAR19 T-cells from NK cell rejection according to HLA-I expression. D. Host NK cells (effector cells, E) were co-cultured with graft miCAR19 T-cells (target cells, T) over a range of E: T ratios. Untransduced graft T-cells and miCAR 19-allo T-cells with full expression of HLA-I were not lysed, while CAR19-allo T-cells with 95% silencing of HLA-I were the most sensitive to NK cell mediated cytotoxicity at all E: T ratios. Notably, graft miCAR19 T-cells with tuned HLA-I silencing of 80-90% remained largely protected from NK cell mediated cytotoxicity. E. Host NK cells (effector cells, E) were co-cultured with graft miCAR19 T-cells (target cells, T) over a range of E: T ratios. Host NK cells most prominently rejected graft T-cells with HLA-I silencing over the first 24 hours of co-culture (E:T ratio of 5:1). All assays were performed with n=3 NK cell donors. F. Rejection of CD19 CAR T-cells by primed CD8 T or NK cells correlates with HLA-ABC levels, in vitro. CD8+ T-cells, from 3 different donors, were first primed with CD19 CAR T-cells and then plated with CD19 CAR T-cells expressing different levels of HLA-ABC (graft cells) in a 1:1 ratio. The number of remaining graft cells were analyzed after 6 days of co-culture. G. Rejection of CD19 CAR T-cells by primed CD8 T or NK cells correlates with HLA-ABC levels, in vitro. NK cells, from 3 different donors, were plated with CD19 CAR T-cells expressing different levels of HLA-ABC (graft cells) in a 5:1 ratio. The number of remaining graft cells were analyzed after 48 h of co-culture.

FIG. 21: TCR-silenced CAR T-cells are not activated in CD3 stimulation assays. T-cells were stimulated with anti-CD3 antibody (OKT3) over a concentration range of 0-17.5 ug/mL, and 24 hours later assessed for expression levels of the CD137/CD69 activation markers. Untransduced T-cells and control CAR T-cells (278) were shown to be activated by OKT3 from 0.54 ug/mL and increased equivalently. Notably, activation marker expression remained unchanged over this same concentration range for TCR-silenced CAR T-cell populations, confirming the loss of TCR functionality upon silencing of the receptor. UTD: Untransduced T-Cells; 278: Scrambled CAR T-Cells; 296: CAR 19 with TCR silencing; 294: CAR19 with TCR and HLA-I silencing (B2M_T5_T5), 5% remaining HLA-I; 293: CAR 19 with TCR and HLA-I silencing (B2M_T5), 10% remaining HLA-I; 292: CAR 19 with TCR and HLA-I silencing (B2M_T2), 20% remaining HLA-I. A. Activation marker expression on the total viable cells (CD8+ CD4+ CAR T-Cells). B. Activation marker expression on CD8+ CAR T-Cell population. C. No cytokine-independent outgrowth of multiplex-engineered miCAR 19 T-cells. Engineered T-cells were cultured either with or without IL-7 and IL-15 over a period of 13 days. Cell survival was observed in all cell populations in the presence of cytokines, but no outgrowth was reported for cells in their absence.

FIG. 22: Proof of principle demonstration of simultaneous HLA-ABC receptor silencing and co-expression of an HLA-E-B2M fusion protein from a single bimodal gene construct. A. All bimodal gene constructs were designed to silence cell surface expression of the TCR via the use of optimized miRNAs targeting CD3z, and to co-express an anti-CD19 CAR (CAR19), HLA-E-B2M fusion protein and the RQR8 reporter gene. Construct pATN302 was designed to additionally silence HLA class I expression using miRNA targeting B2M_T2, while pATN304 included dual miRNAs targeting B2M_T5. B. HEK293 cells were gene modified via lentiviral vector transduction and were analyzed by means of flow cytometry to assess for cell surface expression of HLA class I molecules. Histograms represent membrane expression of HLA-ABC and HLA-E proteins within gene-modified (based on gating of CAR19 positive cells) and unmodified cells. Cells modified with pATN306 express similar levels of HLA-ABC as untransduced cells, but with co-expression of HLA-E-B2M fusion protein. Cells modified with constructs carrying B2M targeting miRNA (pATN302 and pATN304) have downregulated expression of HLA-ABC, while also over-expressing HLA-E. Approximately 60% and 90% silencing of HLA-ABC is shown for cells modified with pATN302 (carrying miRNA targeting B2M_T2) and pATN304 (B2M_T5 dual miRNA), respectively.

FIG. 23: Definitive summary and details of sequences used herein.

FIG. 24: A variety of cell types, including stem cells, progenitor cells or fully differentiated cells, can be used for the creation of universal donor cells according to the present invention. Engineering of these cells can be performed in single gene modification step when using the shown bimodal gene construct. By doing so, universal donor cells are endowed with properties to improve their persistence when infused as a cell therapy product (graft) to patients (host). On the one hand, an optimized miRNA gene silencing cassette is designed to functionally silence the expression of HLA class I and HLA class II molecules. Thus, in a particularly preferred embodiment of the invention, both HLA-I and HLA-II are down-regulated. On the other hand, the same gene construct will allow to express immunomodulatory receptors on the surface of the universal donor cell. Thus, in a preferred embodiment of the invention, CD47 and/or PD-L1 are also up-regulated, preferably both CD47 and PD-L1. In an embodiment of the invention where HLA-I expression is reduced by 50-90%, a non-classical HLA may not be up-regulated. However, in an embodiment where HLA-I expression is reduced by more than 90%, preferably functionally silenced, preferably to a negligible level of expression, a non-classical HLA may be up-regulated, most preferably a modified non-classical HLA sequence such as HLA-E, G or F sequence that is fused to a B2M protein, most preferably wherein the gene encoding the B2M protein is codon-optimised. In this embodiment, the function of the non-classical HLA is to reduce NK-mediated cytotoxicity. In unison, the multiplex engineering of universal cells of the invention aim to limit rejection of these graft cells by the host's immune system.

FIG. 25: Illustration and results of Example 7. A. The three constructs that were used, namely (i) a single hairpin (1 hp) miRNA targeting CD3z (T2), (ii) a dual-hairpin (2 hp) miRNA targeting two different regions of the CD3z transcript (T1_T2), and (iii) a non-targeting miRNA (with scrambled guide strand sequence). B. A pure population of TCR-mCh+ cells was obtained. C. T-cell expansion over production. D. Notably, although equivalent numbers of cells were harvested for each condition, the yield of TCR-silenced T-cells (engineered from the dual-miRNA CD3z_T1_T2) was nearly double when compared to TCR-silenced cells engineered from the single miRNA gene construct targeting CD3z_T2 (Unpaired T-test, p=0.0136). Thus, in a particularly preferred embodiment of the present invention, two miRNA hairpins inhibit CD3z expression, wherein each of the two hairpins targets different regions of the CD3z transcript, preferably wherein the hairpins are CD3z_T1 and CD3z_T2 respectively.

FIG. 26: Induced pluripotent stem cells (iPSCs) transduced with UDC constructs shown in Table 4 of Example 8. UTD: Untransduced iPSCs; 1689: iPSCs transduced with construct silencing 2M_T5 and CIITA_T19, and over-expressing CD47, PDL1, RQR8 and B2M-HLAE; 1690: iPSCs transduced with construct silencing B2M_T5 and CIITA_T19, and over-expressing CD47, PDL1 and RQR8; 1692: iPSCs transduced with construct silencing scr48, and over-expressing CD47, PDL1, RQR8 and B2M-HLAE. HLA-DPDQDR silencing is not assessable in this cell type as these cells do not express these molecules. A. The UDC-transduced cells all express >90% OCT4, indicating that these cells present an undifferentiated phenotype. B. UDC-transduced iPSCs show overexpression of CD47, PD-L1 and HLA-E. C. UDC-transduced iPSCs show overexpression of CD47, PD-L1 and HLA-E, as well as HLA-ABC silencing. D. UDC-transduced iPSCs show overexpression of CD47 and PD-L1, as well as HLA-ABC silencing.

FIG. 27: T-cells transduced with the UDC construct shown in Table 4 of Example 8. UTD: Untransduced T-Cells; Transduced: T-Cells transduced with the UDC. Transduced T-cells showed overexpression of CD47 and PD-L1, as well of HLA-ABC and HLA-DPDQDR silencing.

FIG. 28: Illustration and results of Example 9. A. Overall study design. B. Main study design. C. Flow cytometric analysis to confirm engraftment of modified cells D. Survival curves illustrating complete survival of mice receiving TCR silenced T-cells through 100 days E. Changes in relative body weight percentages, showing that mice receiving the control expanded T-cells trended downwards, while mice receiving TCR silenced T-cells continued to gain weight over the 100 day study period.

FIG. 29: Illustration and results of Example 10, showing sustained and “tuned” silencing of TCR/CD3 and HLA-I in an in vivo model of T-cell engrafted mice. A. In vivo study design in NSG mice. B. Flow cytometric tracking of miCAR19 T-cells from in vivo sampling. Dot plot shows definitive identification of miCAR19 T-cells based on CAR positivity and CD3 silencing. Representative histograms indicate the expression of HLA-I based on sampling from Day 4 and Day 32. Notably, “tuned” silencing of HLA-I was clearly detectable to its varying levels on Day 4 (one day post CAR T-cell infusion), and was sustained until study termination on Day 32 in all sampled tissues.

### DETAILED DESCRIPTION

The present invention relates to allogeneic cells, allogeneic engineered donor cell creation, the use of miRNA gene constructs therein, and universal donor cell (UDC) therapies. In particular, the present invention utilises miRNA-based gene constructs to create allogeneic engineered donor cells in which the expression of one or more cell surface-expressed polypeptides is modulated (e.g. down-regulated and/or up-regulated).

In particular, the present invention relates to engineered donor cells in which the expression of certain combinations of cell surface polypeptides is modulated. In particular, the expression of one or more HLA polypeptides is/are down-regulated by miRNA targeting a HLA polypeptide, such as B2M, or a transcription factor that up-regulates HLA expression. Without wishing to be bound by theory, the present inventors believe that the use of miRNA in this manner is capable of reducing the level of HLA expression to avoid rejection by CD8+ T-cells, but also maintains a level of HLA expression to avoid rejection by NK cells. Thus, engineered donor cells according to the present invention are optimally not rejected by the host immune system, and are hypoimmunogenic. This allows the engineered cells to persist longer, and therefore be more effective in vivo.

In particular, the present invention also relates to engineered donor cells in which the expression of one or more TCR polypeptides is/are down-regulated by miRNA targeting a TCR complex polypeptide, such as CD3z. Without wishing to be bound by theory, the present inventors believe that the use of miRNA in this manner is capable of efficiently and persistently reducing TCR expression to negligible levels, e.g. 0%. Thus, the engineered donor cells of the present invention are allogeneic and can be used as allogeneic CAR T-cells.

### Engineered Donor Cells

In a first aspect, the present invention provides an engineered donor cell with reduced rejection by the immune system of a host, wherein one or more cell surface-expressed polypeptides involved in immune signalling are functionally modulated.

In an embodiment, an engineered donor cell is a cell derived from a donor for use as a therapeutic cell in a cell-based therapy. In an embodiment, the engineered donor cell has been extracted from a donor. In an embodiment, an engineered donor cell is not a cell that occurs in nature. In an embodiment, an engineered donor cell has been modified. In an embodiment, the engineered donor cell comprises a miRNA expression construct. In an embodiment, the engineered donor cell comprises a miRNA expression construct that is not endogenous to the donor cell.

In an embodiment, an engineered donor cell with reduced rejection by the immune system of a host means that the immune response against the engineered donor cell by the immune system of a host is reduced. In an embodiment, the host is a human. In an embodiment, the host is a patient. In an embodiment, the engineered donor cell is allogeneic to the host. In an embodiment, the host CD8+ T-cell response against the engineered donor cell is reduced. In an embodiment, the host NK response against the engineered donor cell is reduced. In an embodiment, the host CD8+ T-cell and NK response against the engineered donor cell is reduced. In an embodiment, the engineered donor cell does not cause GvHD in a host. In an embodiment, the capacity of the engineered donor cell to cause GvHD in a host is reduced. In embodiment, the rejection by the immune system of a host is reduced relative to the rejection of the equivalent unmodified donor cell. In an embodiment, the engineered donor cell is hypoimmunogenic to the host. In an embodiment, the engineered donor cell has improved persistence in the immune system of a host, such as improved persistence relative to the equivalent unmodified donor cell. In an embodiment, the “equivalent unmodified donor cell” is a cell derived from the same source/donor as the engineered donor cell, but has not been modified to comprise a miRNA construct according to the present invention.

In an embodiment, the cell surface-expressed polypeptides involved in immune signalling are polypeptides encoded by the donor cell, which would be expressed on the cell surface of the donor cell. In an embodiment, the encoded polypeptides are targeted for cell surface expression. In particular, in any embodiments wherein polypeptides are up-regulated, the up-regulated polypeptides may be polypeptides that are expressed on the surface of the engineered donor cell. In particular, in any embodiment wherein polypeptides are functionally down-regulated, the down-regulated polypeptides may be polypeptides that are expressed on the surface of the equivalent unmodified donor cell.

In an embodiment, the cell surface-expressed polypeptides involved in immune signalling elicit an immune response from the immune system of a host. In an embodiment, the polypeptides increase the magnitude of an immune response. In an embodiment, the immune response is directed against the engineered donor cell.

In an embodiment, functional modulation comprises modulating the formation of functional complexes that comprise the polypeptides on the surface of the cell. In an embodiment, functional modulation comprises directly modulating the polypeptides. In an embodiment, functional modulation comprises modulating the expression of the polypeptides. In an embodiment, functional modulation comprises modulating the transcription or translation of the genes encoding the polypeptides. Thus, in an embodiment, functional modulation comprises targeting the polypeptide, the mRNA encoding the polypeptide, or the gene encoding the polypeptide. In an embodiment, the modulation is relative to an equivalent donor cell that has not been modified or engineered. In an embodiment, modulation is relative to the equivalent donor cell that does not comprise a non-endogenous miRNA construct. In an embodiment, modulation is relative to the equivalent donor cell that does not comprise a miRNA construct of the invention. In an embodiment, the modulation in the engineered donor cell is permanent.

In an embodiment, one or more cell surface-expressed polypeptides involved in immune signalling are down-regulated. In an embodiment, the polypeptides are functionally down-regulated. In an embodiment, the localisation of the polypeptides to the cell surface is inhibited. In an embodiment, the number of polypeptides on the cell surface is reduced. In an embodiment, the formation of functional complexes on the cell surface that comprise one or more of the polypeptides is inhibited. In an embodiment, the number of functional complexes that comprise one or more of the polypeptides on the cell surface is reduced. In an embodiment, the functional complexes are HLA and/or TCR complexes. In an embodiment, the formation of HLA and/or TCR complexes is inhibited.

In an embodiment, the down-regulation of the one or more cell-surface expressed polypeptides involved in immune signalling is achieved by inhibiting the expression of a target gene. In an embodiment, the down-regulation of the one or more cell-surface expressed polypeptides involved in immune signalling is achieved by silencing a target gene. In an embodiment, the expression of the target gene is reduced relative to the equivalent unmodified donor cell. It will be understood that, in the context of the present invention, all reductions in e.g. expression or inhibition of polypeptides and genes within an engineered donor cell can be considered as relative to the equivalent unmodified donor cell. In a general embodiment, the expression of target genes is reduced to 70% or less, 60% or less, 50% or less, 40% or less, 30% or less, 20% or less, 10% or less, or 0% or a negligible amount. In a preferred embodiment, the down-regulation involves a tuning that maintains a reduced level of expression capable of improving the persistence of the engineered donor cell in the immune system of a host, such as by inhibiting NK cell killing of the engineered donor cell. Accordingly, in an embodiment the expression of target genes is not reduced to less than 30%, less than 20%, or less than 10%.

In an embodiment, the down-regulation of the one or more cell-surface expressed polypeptides involved in immune signalling is achieved by miRNA inhibition of a target gene. In an embodiment, the miRNA comprises a sequence that binds complementary to the mRNA transcript of the target gene, thereby inhibiting the expression of the target gene. In an embodiment, the down-regulation is achieved by miRNA inhibiting the expression of a gene encoding the cell surface-expressed polypeptide. In an embodiment, the down-regulation is achieved by miRNA inhibiting the expression of a transcription factor that induces the expression of a gene encoding the cell surface-expressed polypeptide.

In an embodiment, the down-regulation of a cell-surface expressed polypeptide involved in immune signalling is achieved by a miRNA construct comprising a single miRNA hairpin targeting a gene encoding the polypeptide. In an embodiment, the down-regulation is achieved by a miRNA construct comprising two miRNA hairpins targeting a gene encoding the polypeptide. In an embodiment, the down-regulation is achieved by a miRNA construct comprising three miRNA hairpins targeting a gene encoding the polypeptide. In an embodiment where there are more than one miRNA hairpin targeting the same gene, the miRNA hairpins may target different transcript sequences comprised in the mRNA transcript that is encoded by that gene. In an embodiment, inhibition by miRNA is permanent in the engineered donor cells.

In an embodiment, one or more of the down-regulated surface-expressed polypeptides are selected from the group consisting of HLA class I (HLA-I) polypeptides and HLA class II (HLA-II) polypeptides. In an embodiment, one or more of the down-regulated surface-expressed polypeptides are polypeptides required for functional HLA class I and/or HLA class II formation. Thus, in an embodiment, a HLA is down-regulated. In an embodiment, HLA-I is down-regulated. In an embodiment, HLA-II is down-regulated. In an embodiment, both HLA-I and HLA-II are down-regulated.

In an embodiment, the down-regulation of HLA class I polypeptides is achieved by miRNA inhibiting the expression of one or more of B2M (beta-2-microglobulin), NLRC5 (NLR family CARD domain containing 5), TAP1, TAP2, TAPBP, RFX5 (regulatory factor X5), RFXANK (regulatory factor X associated ankyrin containing protein), and/or RFXAP (regulatory factor X associated protein).

In an embodiment, the down-regulation of HLA class I polypeptides is achieved by miRNA inhibiting the expression of B2M. In an embodiment, the miRNA targets a sequence having at least 80%, 85%, 90%, 95%, 96%, 97%, 98%, 99% or 100% identity to TTGACTTTCCATTCTCTGCTGG (SEQ ID NO: 1; B2M_T2). In an embodiment, the miRNA comprises a sequence complementary to a sequence having at least 80%, 85%, 90%, 95%, 96%, 97%, 98%, 99% or 100% identity to TTGACTTTCCATTCTCTGCTGG (SEQ ID NO: 1; B2M_T2). In an embodiment, the miRNA targets a sequence having at least 80%, 85%, 90%, 95%, 96%, 97%, 98%, 99% or 100% identity to TTATGCACGCTTAACTATCTTA (SEQ ID NO: 2; B2M_T3). In an embodiment, the miRNA comprises a sequence complementary to a sequence having at least 80%, 85%, 90%, 95%, 96%, 97%, 98%, 99% or 100% identity to TTATGCACGCTTAACTATCTTA (SEQ ID NO: 2; B2M_T3). In an embodiment, the miRNA targets a sequence having at least 80%, 85%, 90%, 95%, 96%, 97%, 98%, 99% or 100% identity to TAAACCTGAATCTTTGGAGTAC (SEQ ID NO: 3; B2M_T5). In an embodiment, the miRNA comprises a sequence complementary to a sequence having at least 80%, 85%, 90%, 95%, 96%, 97%, 98%, 99% or 100% identity to TAAACCTGAATCTTTGGAGTAC (SEQ ID NO: 3; B2M_T5). In an embodiment, the miRNA targets a sequence having at least 80%, 85%, 90%, 95%, 96%, 97%, 98%, 99% or 100% identity to CCAGCAGAGAATGGAAAGTCAA (SEQ ID NO: 30; B2M_T2). In an embodiment, the miRNA comprises a sequence complementary to a sequence having at least 80%, 85%, 90%, 95%, 96%, 97%, 98%, 99% or 100% identity to CCAGCAGAGAATGGAAAGTCAA (SEQ ID NO: 30; B2M_T2). In an embodiment, the miRNA targets a sequence having at least 80%, 85%, 90%, 95%, 96%, 97%, 98%, 99% or 100% identity to GTACTCCAAAGATTCAGGTTTA (SEQ ID NO: 31; B2M_T5). In an embodiment, the miRNA comprises a sequence complementary to a sequence having at least 80%, 85%, 90%, 95%, 96%, 97%, 98%, 99% or 100% identity to GTACTCCAAAGATTCAGGTTTA (SEQ ID NO: 31; B2M_T5). In an embodiment, the miRNA targets a sequence having at least 80%, 85%, 90%, 95%, 96%, 97%, 98%, 99% or 100% identity to CGTGCATAAGTTAACTTCCAAT (SEQ ID NO: 32; B2M_T6). In an embodiment, the miRNA comprises a sequence complementary to a sequence having at least 80%, 85%, 90%, 95%, 96%, 97%, 98%, 99% or 100% identity to CGTGCATAAGTTAACTTCCAAT (SEQ ID NO: 32; B2M_T6). In an embodiment, the miRNA targets a sequence having at least 80%, 85%, 90%, 95%, 96%, 97%, 98%, 99% or 100% identity to GCTGTCTCCATGTTTGATGTAT (SEQ ID NO: 33; B2M_T7). In an embodiment, the miRNA comprises a sequence complementary to a sequence having at least 80%, 85%, 90%, 95%, 96%, 97%, 98%, 99% or 100% identity to GCTGTCTCCATGTTTGATGTAT (SEQ ID NO: 33; B2M_T7). In an embodiment, the miRNA targets a sequence having at least 80%, 85%, 90%, 95%, 96%, 97%, 98%, 99% or 100% identity to GGTTGTGGTTAATCTGGTTTAT (SEQ ID NO: 34; B2M_T8). In an embodiment, the miRNA comprises a sequence complementary to a sequence having at least 80%, 85%, 90%, 95%, 96%, 97%, 98%, 99% or 100% identity to GGTTGTGGTTAATCTGGTTTAT (SEQ ID NO: 34; B2M_T8). In an embodiment, the miRNA targets a sequence having at least 80%, 85%, 90%, 95%, 96%, 97%, 98%, 99% or 100% identity to CTCTGCTTAGAATTTGGGGGAA (SEQ ID NO: 35; B2M_T9). In an embodiment, the miRNA comprises a sequence complementary to a sequence having at least 80%, 85%, 90%, 95%, 96%, 97%, 98%, 99% or 100% identity to CTCTGCTTAGAATTTGGGGGAA (SEQ ID NO: 35; B2M_T9). In an embodiment, the miRNA targets a sequence having at least 80%, 85%, 90%, 95%, 96%, 97%, 98%, 99% or 100% identity to CATCCGACATTGAAGTTGACTT (SEQ ID NO: 36; B2M_T10). In an embodiment, the miRNA comprises a sequence complementary to a sequence having at least 80%, 85%, 90%, 95%, 96%, 97%, 98%, 99% or 100% identity to CATCCGACATTGAAGTTGACTT (SEQ ID NO: 36; B2M_T10). In an embodiment, the miRNA targets a sequence having at least 80%, 85%, 90%, 95%, 96%, 97%, 98%, 99% or 100% identity to CCAGCGTACTCCAAAGATTCAG (SEQ ID NO: 37; B2M_T11). In an embodiment, the miRNA comprises a sequence complementary to a sequence having at least 80%, 85%, 90%, 95%, 96%, 97%, 98%, 99% or 100% identity to CCAGCGTACTCCAAAGATTCAG (SEQ ID NO: 37; B2M_T11). In an embodiment, the miRNA targets a sequence having at least 80%, 85%, 90%, 95%, 96%, 97%, 98%, 99% or 100% identity to CCCCACTGAAAAAGATGAGTAT (SEQ ID NO: 38; B2M_T12). In an embodiment, the miRNA comprises a sequence complementary to a sequence having at least 80%, 85%, 90%, 95%, 96%, 97%, 98%, 99% or 100% identity to CCCCACTGAAAAAGATGAGTAT (SEQ ID NO: 38; B2M_T12). In an embodiment, the miRNA targets a sequence having at least 80%, 85%, 90%, 95%, 96%, 97%, 98%, 99% or 100% identity to CGTACTCCAAAGATTCAGGTTT (SEQ ID NO: 39; B2M_T13). In an embodiment, the miRNA comprises a sequence complementary to a sequence having at least 80%, 85%, 90%, 95%, 96%, 97%, 98%, 99% or 100% identity to CGTACTCCAAAGATTCAGGTTT (SEQ ID NO: 39; B2M_T13). In an embodiment, the miRNA targets a sequence having at least 80%, 85%, 90%, 95%, 96%, 97%, 98%, 99% or 100% identity to AAGGCATGGTTGTGGTTAATCT (SEQ ID NO: 40; B2M_T14). In an embodiment, the miRNA comprises a sequence complementary to a sequence having at least 80%, 85%, 90%, 95%, 96%, 97%, 98%, 99% or 100% identity to AAGGCATGGTTGTGGTTAATCT (SEQ ID NO: 40; B2M_T14). In an embodiment, the miRNA targets a sequence having at least 80%, 85%, 90%, 95%, 96%, 97%, 98%, 99% or 100% identity to GACTGGTCTTTCTATCTCTTGT (SEQ ID NO: 41; B2M_T15). In an embodiment, the miRNA comprises a sequence complementary to a sequence having at least 80%, 85%, 90%, 95%, 96%, 97%, 98%, 99% or 100% identity to GACTGGTCTTTCTATCTCTTGT (SEQ ID NO: 41; B2M_T15). In an embodiment, the miRNA targets a sequence having at least 80%, 85%, 90%, 95%, 96%, 97%, 98%, 99% or 100% identity to GATCGAGACATGTAAGCAGCAT (SEQ ID NO: 42; B2M_T16). In an embodiment, the miRNA comprises a sequence complementary to a sequence having at least 80%, 85%, 90%, 95%, 96%, 97%, 98%, 99% or 100% identity to GATCGAGACATGTAAGCAGCAT (SEQ ID NO: 42; B2M_T16). In an embodiment, the miRNA targets a sequence having at least 80%, 85%, 90%, 95%, 96%, 97%, 98%, 99% or 100% identity to TTGCTATGTGTCTGGGTTTCAT (SEQ ID NO: 43; B2M_T17). In an embodiment, the miRNA comprises a sequence complementary to a sequence having at least 80%, 85%, 90%, 95%, 96%, 97%, 98%, 99% or 100% identity to TTGCTATGTGTCTGGGTTTCAT (SEQ ID NO: 43; B2M_T17). In an embodiment, the miRNA targets a sequence having at least 80%, 85%, 90%, 95%, 96%, 97%, 98%, 99% or 100% identity to TAAGATAGTTAAGCGTGCATAA (SEQ ID NO: 44; B2M_T3). In an embodiment, the miRNA comprises a sequence complementary to a sequence having at least 80%, 85%, 90%, 95%, 96%, 97%, 98%, 99% or 100% identity to TAAGATAGTTAAGCGTGCATAA (SEQ ID NO: 44; B2M_T3).

NLRC5, TAP1, TAP2, TAPBP, RFX5, RFXANK, and RFXAP are transcription factors that induce the expression of HLA-I. In an embodiment, the down-regulation of HLA-I is achieved by miRNA inhibiting the expression of a transcription factor that induces the expression of HLA-I.

In an embodiment, the down-regulation of HLA class I polypeptides is achieved by miRNA inhibiting the expression of NLRC5. In an embodiment, the miRNA targets a sequence having at least 80%, 85%, 90%, 95%, 96%, 97%, 98%, 99% or 100% identity to TTTGCATAGAAGATAACCTTCC (SEQ ID NO: 4; NLRC_T4). In an embodiment, the miRNA comprises a sequence complementary to a sequence having at least 80%, 85%, 90%, 95%, 96%, 97%, 98%, 99% or 100% identity to TTTGCATAGAAGATAACCTTCC (SEQ ID NO: 4; NLRC_4). In an embodiment, the miRNA targets a sequence having at least 80%, 85%, 90%, 95%, 96%, 97%, 98%, 99% or 100% identity to TTAGTCTGTGAGTAAGCAAGGC (SEQ ID NO: 5; NLRC_T9). In an embodiment, the miRNA comprises a sequence complementary to a sequence having at least 80%, 85%, 90%, 95%, 96%, 97%, 98%, 99% or 100% identity to TTAGTCTGTGAGTAAGCAAGGC (SEQ ID NO: 5; NLRC_T9). In an embodiment, the miRNA targets a sequence having at least 80%, 85%, 90%, 95%, 96%, 97%, 98%, 99% or 100% identity to TATAGACCAACAATCATGTATC (SEQ ID NO: 6; NLRC_T11). In an embodiment, the miRNA comprises a sequence complementary to a sequence having at least 80%, 85%, 90%, 95%, 96%, 97%, 98%, 99% or 100% identity to TATAGACCAACAATCATGTATC (SEQ ID NO: 6; NLRC_T11). In an embodiment, the miRNA targets a sequence having at least 80%, 85%, 90%, 95%, 96%, 97%, 98%, 99% or 100% identity to TGAAAGCATAGCCTGTCTGCTG (SEQ ID NO: 7; NLRC_T16). In an embodiment, the miRNA comprises a sequence complementary to a sequence having at least 80%, 85%, 90%, 95%, 96%, 97%, 98%, 99% or 100% identity to TGAAAGCATAGCCTGTCTGCTG (SEQ ID NO: 7; NLRC_T16). In an embodiment, the miRNA targets a sequence having at least 80%, 85%, 90%, 95%, 96%, 97%, 98%, 99% or 100% identity to GGAAGGTTATCTTCTATGCAAA (SEQ ID NO: 45; NLRC_T4). In an embodiment, the miRNA comprises a sequence complementary to a sequence having at least 80%, 85%, 90%, 95%, 96%, 97%, 98%, 99% or 100% identity to GGAAGGTTATCTTCTATGCAAA (SEQ ID NO: 45; NLRC_T4). In an embodiment, the miRNA targets a sequence having at least 80%, 85%, 90%, 95%, 96%, 97%, 98%, 99% or 100% identity to GCCTTGCTTACTCACAGACTAA (SEQ ID NO: 46; NLRC_T9). In an embodiment, the miRNA comprises a sequence complementary to a sequence having at least 80%, 85%, 90%, 95%, 96%, 97%, 98%, 99% or 100% identity to GCCTTGCTTACTCACAGACTAA (SEQ ID NO: 46; NLRC_T9). In an embodiment, the miRNA targets a sequence having at least 80%, 85%, 90%, 95%, 96%, 97%, 98%, 99% or 100% identity to GATACATGATTGTTGGTCTATA (SEQ ID NO: 47; NLRC_T11). In an embodiment, the miRNA comprises a sequence complementary to a sequence having at least 80%, 85%, 90%, 95%, 96%, 97%, 98%, 99% or 100% identity to GATACATGATTGTTGGTCTATA (SEQ ID NO: 47; NLRC_T11). In an embodiment, the miRNA targets a sequence having at least 80%, 85%, 90%, 95%, 96%, 97%, 98%, 99% or 100% identity to CAGCAGACAGGCTATGCTTTCA (SEQ ID NO: 48; NLRC_T16). In an embodiment, the miRNA comprises a sequence complementary to a sequence having at least 80%, 85%, 90%, 95%, 96%, 97%, 98%, 99% or 100% identity to CAGCAGACAGGCTATGCTTTCA (SEQ ID NO: 48; NLRC_T16).

In an embodiment, the down-regulation of HLA class I polypeptides is achieved by miRNA inhibiting the expression of TAP1. In an embodiment, the down-regulation of HLA class I polypeptides is achieved by miRNA inhibiting the expression of TAP2. In an embodiment, the down-regulation of HLA class I polypeptides is achieved by miRNA inhibiting the expression of TAPBP. In an embodiment, the down-regulation of HLA class I polypeptides is achieved by miRNA inhibiting the expression of RFX5. In an embodiment, the down-regulation of HLA class I polypeptides is achieved by miRNA inhibiting the expression of RFXANK. In an embodiment, the down-regulation of HLA class I polypeptides is achieved by miRNA inhibiting the expression of RFXAP.

In a preferred embodiment, the endogenous HLA Class I of the donor cell is down-regulated. Accordingly, in an embodiment, the engineered donor cell has reduced immunogenicity. In an embodiment, HLA Class I expression is reduced to 50% or less, 40% or less, 30% or less, 20% or less, 10% or less, 5% or less, 0% or a negligible level of expression. In an embodiment, reduced immunogenicity means that the engineered donor cell elicits a reduced level of rejection by the immune system of a subject or patient. In an embodiment, the engineered donor cell elicits a reduced level of rejection or killing by CD8+ T-cells in the immune system of a subject or patient. In an embodiment, reduced immunogenicity means that the engineered donor cells are more persistent in an unmatched host. In the context of the present invention “killing” refers to the killing of the engineered donor cells.

However, the present inventors have also surprisingly found that miRNA can be used to down-regulate HLA Class I polypeptides such as B2M in donor cells (for the advantages of reduced immunogenicity noted above), whilst still advantageously maintaining a (reduced) level of HLA Class I expression. This may be referred to as B2M “tuning”. Thus, in an embodiment, the level of HLA Class I expression maintained in the engineered donor cells is sufficient to inhibit killing by NK cells. In an embodiment, the reduction in HLA Class I expression in the engineered donor cells is sufficient to inhibit killing by CD8+ cells, but the level of HLA Class I expression maintained in the engineered donor cells is still sufficient to inhibit killing by NK cells. In an embodiment, the engineered donor cells of the invention avoid killing by both CD8+ T-cells and NK cells. In an embodiment, the CD8+ T-cells and NK cells are those of the host/patient immune system. In an embodiment, the engineered donor cells of the invention elicit reduced CD8+ T-cell mediated cytotoxicity and reduced NK cell mediated cytotoxicity. Accordingly, in an embodiment, HLA Class I expression is reduced by an amount that is between 50% and 95% inclusive, preferably between 50% and 90% inclusive, preferably between 70% and 95% inclusive, most preferably between 70% and 90% inclusive. In a preferred form of such an embodiment, the engineered donor cell does not comprise an up-regulated genetically modified non-classical HLA polypeptide.

In an embodiment, the down-regulation of HLA class II polypeptides is achieved by miRNA inhibiting the expression of one or more of CIITA (class II major histocompatibility complex transactivator), RFX5, RFXANK, and/or RFXAP.

CIITA, RFX5, RFXANK, and RFXAP are transcription factors that induce the expression of HLA-II. In an embodiment, the down-regulation of HLA-II is achieved by miRNA inhibiting the expression of a transcription factor that induces the expression of HLA-II.

In an embodiment, the down-regulation of HLA class II polypeptides is achieved by miRNA inhibiting the expression of CIITA. In an embodiment, the miRNA targets a sequence having at least 80%, 85%, 90%, 95%, 96%, 97%, 98%, 99% or 100% identity to TTTCCAAGGACTTCAGCTGGGG (SEQ ID NO: 8; CIITA_T13). In an embodiment, the miRNA comprises a sequence complementary to a sequence having at least 80%, 85%, 90%, 95%, 96%, 97%, 98%, 99% or 100% identity to TTTCCAAGGACTTCAGCTGGGG (SEQ ID NO: 8; CIITA_T13). In an embodiment, the miRNA targets a sequence having at least 80%, 85%, 90%, 95%, 96%, 97%, 98%, 99% or 100% identity to TTAGTGTCCTCAGAGAACATGC (SEQ ID NO: 9; CIITA_T16). In an embodiment, the miRNA comprises a sequence complementary to a sequence having at least 80%, 85%, 90%, 95%, 96%, 97%, 98%, 99% or 100% identity to TTAGTGTCCTCAGAGAACATGC (SEQ ID NO: 9; CIITA_T16). In an embodiment, the miRNA targets a sequence having at least 80%, 85%, 90%, 95%, 96%, 97%, 98%, 99% or 100% identity to TATTGTACAAGCTTAGCCTGAG (SEQ ID NO: 10; CIITA_T19). In an embodiment, the miRNA comprises a sequence complementary to a sequence having at least 80%, 85%, 90%, 95%, 96%, 97%, 98%, 99% or 100% identity to TATTGTACAAGCTTAGCCTGAG (SEQ ID NO: 10; CIITA_T19). In an embodiment, the miRNA targets a sequence having at least 80%, 85%, 90%, 95%, 96%, 97%, 98%, 99% or 100% identity to CCCCAGCTGAAGTCCTTGGAAA (SEQ ID NO: 49; CIITA_T13). In an embodiment, the miRNA comprises a sequence complementary to a sequence having at least 80%, 85%, 90%, 95%, 96%, 97%, 98%, 99% or 100% identity to CCCCAGCTGAAGTCCTTGGAAA (SEQ ID NO: 49; CIITA_T13). In an embodiment, the miRNA targets a sequence having at least 80%, 85%, 90%, 95%, 96%, 97%, 98%, 99% or 100% identity to GCATGTTCTCTGAGGACACTAA (SEQ ID NO: 50; CIITA_T16). In an embodiment, the miRNA comprises a sequence complementary to a sequence having at least 80%, 85%, 90%, 95%, 96%, 97%, 98%, 99% or 100% identity to GCATGTTCTCTGAGGACACTAA (SEQ ID NO: 50; CIITA_T16). In an embodiment, the miRNA targets a sequence having at least 80%, 85%, 90%, 95%, 96%, 97%, 98%, 99% or 100% identity to CTCAGGCTAAGCTTGTACAATA (SEQ ID NO: 51; CIITA_T19). In an embodiment, the miRNA comprises a sequence complementary to a sequence having at least 80%, 85%, 90%, 95%, 96%, 97%, 98%, 99% or 100% identity to CTCAGGCTAAGCTTGTACAATA (SEQ ID NO: 51; CIITA_T19).

In an embodiment, the down-regulation of HLA class II polypeptides is achieved by miRNA inhibiting the expression of RFX5. In an embodiment, the down-regulation of HLA class II polypeptides is achieved by miRNA inhibiting the expression of RFXANK. In an embodiment, the down-regulation of HLA class II polypeptides is achieved by miRNA inhibiting the expression of RFXAP.

In a preferred embodiment, the endogenous HLA Class II of the donor cell is down-regulated. Accordingly, in an embodiment, the engineered donor cell has reduced immunogenicity. In an embodiment, HLA Class II expression is reduced to 50% or less, 40% or less, 30% or less, 20% or less, 10% or less, 5% or less, 0% or a negligible level of expression. In an embodiment, reduced immunogenicity means that the engineered donor cell elicits a reduced level of rejection by the immune system of a subject or patient. In an embodiment, the engineered donor cell elicits a reduced level of rejection or killing by CD4+ T-cells in the immune system of a subject or patient. In an embodiment, reduced immunogenicity means that the engineered donor cells are more persistent in an unmatched host. In the context of the present invention “killing” refers to the killing of the engineered donor cells.

In an embodiment, the reduction in HLA Class II expression in the engineered donor cells is sufficient to inhibit killing by CD4+ cells. In an embodiment, the CD4+ T-cells are those of the host/patient immune system. In an embodiment, the engineered donor cells of the invention elicit reduced CD4+ T-cell mediated cytotoxicity. In a preferred embodiment, down-regulation of HLA Class II expression to reduce CD4+ killing is combined with down-regulation of HLA class I expression to reduce CD8+ killing. In a preferred embodiment, this is further combined with using miRNA to reduce HLA class I expression while also maintaining a low level of HLA class I expression to reduce NK killing. In an alternative preferred embodiment, this is further combined with effective functional silencing of HLA class I expression, i.e. to a negligible level of expression/function and up-regulation of a non-classical HLA polypeptide, preferably a HLA-E, F or G polypeptide fused to a B2M polypeptide according to the present invention.

In an embodiment of the engineered donor cell of the invention, one or more of the down-regulated surface-expressed polypeptides are T-cell receptor (TCR) polypeptides. In an embodiment, one or more of the polypeptides form part of the TCR-CD3 complex. In an embodiment, one or more of the polypeptides are associated with the TCR-CD3 complex. In an embodiment, one or more of the polypeptides are required for formation of a functional TCR complex. Accordingly, in an embodiment, the TCR-CD3 complex is down-regulated.

In an embodiment, the down-regulation of a TCR polypeptide is achieved by miRNA inhibiting the expression of TCRa (TCRα, TRAC), TCRb (TCRβ), CD3g (CD3γ), CD3d (CD3δ), CD3e (CD3ε), and/or CD3z (CD3ζ).

In a most preferred embodiment, the down-regulation of a TCR polypeptide is achieved by miRNA inhibiting the expression of CD3z. In an embodiment, the miRNA targets a sequence having at least 80%, 85%, 90%, 95%, 96%, 97%, 98%, 99% or 100% identity to TTTGGAGCTAAATATAACCAAA (SEQ ID NO: 11; CD3z_T1). In an embodiment, the miRNA comprises a sequence complementary to a sequence having at least 80%, 85%, 90%, 95%, 96%, 97%, 98%, 99% or 100% identity to TTTGGAGCTAAATATAACCAAA (SEQ ID NO: 11; CD3z_T1). In an embodiment, the miRNA targets a sequence having at least 80%, 85%, 90%, 95%, 96%, 97%, 98%, 99% or 100% identity to TATCCTAGTACATTGACGGGTT (SEQ ID NO: 12; CD3z_T2). In an embodiment, the miRNA comprises a sequence complementary to a sequence having at least 80%, 85%, 90%, 95%, 96%, 97%, 98%, 99% or 100% identity to TATCCTAGTACATTGACGGGTT (SEQ ID NO: 12; CD3z_T2). In an embodiment, the miRNA targets a sequence having at least 80%, 85%, 90%, 95%, 96%, 97%, 98%, 99% or 100% identity to TTCCACTTCATCTTGTCCTTTC (SEQ ID NO: 13; CD3z_T3). In an embodiment, the miRNA comprises a sequence complementary to a sequence having at least 80%, 85%, 90%, 95%, 96%, 97%, 98%, 99% or 100% identity to TTCCACTTCATCTTGTCCTTTC (SEQ ID NO: 13; CD3z_T3). In an embodiment, the miRNA targets a sequence having at least 80%, 85%, 90%, 95%, 96%, 97%, 98%, 99% or 100% identity to TTTGGTTATATTTAGCTCCAAA (SEQ ID NO: 52; CD3z_T1). In an embodiment, the miRNA comprises a sequence complementary to a sequence having at least 80%, 85%, 90%, 95%, 96%, 97%, 98%, 99% or 100% identity to TTTGGTTATATTTAGCTCCAAA (SEQ ID NO: 52; CD3z_T1). In an embodiment, the miRNA targets a sequence having at least 80%, 85%, 90%, 95%, 96%, 97%, 98%, 99% or 100% identity to AACCCGTCAATGTACTAGGATA (SEQ ID NO: 53; CD3z_T2). In an embodiment, the miRNA comprises a sequence complementary to a sequence having at least 80%, 85%, 90%, 95%, 96%, 97%, 98%, 99% or 100% identity to AACCCGTCAATGTACTAGGATA (SEQ ID NO: 53; CD3z_T2). In an embodiment, the miRNA targets a sequence having at least 80%, 85%, 90%, 95%, 96%, 97%, 98%, 99% or 100% identity to GAAAGGACAAGATGAAGTGGAA (SEQ ID NO: 54; CD3z_T3). In an embodiment, the miRNA comprises a sequence complementary to a sequence having at least 80%, 85%, 90%, 95%, 96%, 97%, 98%, 99% or 100% identity to GAAAGGACAAGATGAAGTGGAA (SEQ ID NO: 54; CD3z_T3).

In an embodiment, the down-regulation of a TCR polypeptide is achieved by miRNA inhibiting the expression of TCRa. In an embodiment, the miRNA targets a sequence having at least 80%, 85%, 90%, 95%, 96%, 97%, 98%, 99% or 100% identity to TCATGAGCAGATTAAACCCGGC (SEQ ID NO: 14; TRAC_T1). In an embodiment, the miRNA comprises a sequence complementary to a sequence having at least 80%, 85%, 90%, 95%, 96%, 97%, 98%, 99% or 100% identity to TCATGAGCAGATTAAACCCGGC (SEQ ID NO: 14; TRAC_T1). In an embodiment, the miRNA targets a sequence having at least 80%, 85%, 90%, 95%, 96%, 97%, 98%, 99% or 100% identity to TTAGGTTCGTATCTGTTTCAAA (SEQ ID NO: 15; TRAC_T4). In an embodiment, the miRNA comprises a sequence complementary to a sequence having at least 80%, 85%, 90%, 95%, 96%, 97%, 98%, 99% or 100% identity to TTAGGTTCGTATCTGTTTCAAA (SEQ ID NO: 15; TRAC_T4). In an embodiment, the miRNA targets a sequence having at least 80%, 85%, 90%, 95%, 96%, 97%, 98%, 99% or 100% identity to TCAGATTTGTTGCTCCAGGCCA (SEQ ID NO: 15; TRAC_T5). In an embodiment, the miRNA comprises a sequence complementary to a sequence having at least 80%, 85%, 90%, 95%, 96%, 97%, 98%, 99% or 100% identity to TCAGATTTGTTGCTCCAGGCCA (SEQ ID NO: 15; TRAC_T5). In an embodiment, the miRNA targets a sequence having at least 80%, 85%, 90%, 95%, 96%, 97%, 98%, 99% or 100% identity to GCCGGGTTTAATCTGCTCATGA (SEQ ID NO: 55; TRAC_T1). In an embodiment, the miRNA comprises a sequence complementary to a sequence having at least 80%, 85%, 90%, 95%, 96%, 97%, 98%, 99% or 100% identity to GCCGGGTTTAATCTGCTCATGA (SEQ ID NO: 55; TRAC_T1). In an embodiment, the miRNA targets a sequence having at least 80%, 85%, 90%, 95%, 96%, 97%, 98%, 99% or 100% identity to TTTGAAACAGATACGAACCTAA (SEQ ID NO: 56; TRAC_T4). In an embodiment, the miRNA comprises a sequence complementary to a sequence having at least 80%, 85%, 90%, 95%, 96%, 97%, 98%, 99% or 100% identity to TTTGAAACAGATACGAACCTAA (SEQ ID NO: 56; TRAC_T4). In an embodiment, the miRNA targets a sequence having at least 80%, 85%, 90%, 95%, 96%, 97%, 98%, 99% or 100% identity to TGGCCTGGAGCAACAAATCTGA (SEQ ID NO: 57; TRAC_T5). In an embodiment, the miRNA comprises a sequence complementary to a sequence having at least 80%, 85%, 90%, 95%, 96%, 97%, 98%, 99% or 100% identity to TGGCCTGGAGCAACAAATCTGA (SEQ ID NO: 57; TRAC_T5).

In an embodiment, the down-regulation of a TCR polypeptide is achieved by miRNA inhibiting the expression of TCRb. In an embodiment, the down-regulation of a TCR polypeptide is achieved by miRNA inhibiting the expression of CD3g. In an embodiment, the down-regulation of a TCR polypeptide is achieved by miRNA inhibiting the expression of CD3d. In an embodiment, the down-regulation of a TCR polypeptide is achieved by miRNA inhibiting the expression of CD3e.

In a preferred embodiment, the endogenous TCR of the engineered donor cell is down-regulated. In an embodiment, this cell is a T-cell. In an embodiment, this cell is a CAR T-cell. Accordingly, in an embodiment, the engineered donor cell is allogeneic, such as an allogeneic CAR T-cell. In an embodiment, TCR expression is reduced to 70% or less, 60% or less, 50% or less, 40% or less, 30% or less, 20% or less, 10% or less, 5% or less, 0% or a negligible level of expression. In an embodiment, the endogenous TCR of the engineered donor cell is not functionally expressed. In an embodiment, the engineered donor cells elicit a reduced level of alloreactivity in mixed lymphocyte reactions when cultured with unmatched peripheral blood mononuclear cells (PBMCs). In an embodiment, unmatched means that the PBMCs are derived from a different host to the engineered donor cell. In an embodiment, unmatched means that the PBMCs are allogeneic to the engineered donor cells. In an embodiment, the engineered donor cells elicit a reduced level of CD137 expression in CD8+ CAR T-cells when cultured with unmatched PBMCs. In an embodiment, the level of CD137 expression is reduced to as little as 40%, 30%, 20%, 10%, 5%, 0% or a negligible level of expression. In an embodiment, the engineered donor cell is non-alloreactive. In an embodiment, the reduction is relative to the mixed lymphocyte reactions elicited when the equivalent unmodified donor cells are cultured with unmatched PBMCs. In an embodiment, the engineered donor cell is suitable for allogeneic administration. In an embodiment, the engineered donor cell is suitable for use in allogeneic CAR T-cell therapy.

In a particularly preferred embodiment of the engineered donor cell of the present invention, a classical HLA polypeptide is down-regulated and a TCR polypeptide is down-regulated. In preferred embodiments of this type, the HLA polypeptide is an endogenous HLA class I and/or II polypeptide. Accordingly, in an embodiment, the resulting engineered donor cell has reduced immunogenicity as defined above, and is suitable for allogeneic administration as defined above.

In an embodiment of the engineered donor cell of the present invention, one or more of the down-regulated surface-expressed polypeptides are a CD58 polypeptide. In an embodiment, the down-regulation of a CD58 polypeptide is achieved by miRNA targeting the expression of CD58. In an embodiment, the down-regulation of a CD58 polypeptide is achieved by miRNA inhibiting the expression of CD58. In an embodiment, CD58 is down-regulated to reduce bystander T-cell activation. In an embodiment, CD58 is down-regulated to limit co-activation of bystander T-cells. In an embodiment, CD58 is down-regulated to reduce rejection by host CD4+ and CD8+ T-cells.

In any embodiment of the invention wherein the engineered donor cell comprises a miRNA expression construct targeting one or more of the B2M, CIITA, NLRC5, CD3z and TRAC genes, the transcripts of these respective genes that are targeted by the miRNA expression construct can be any of the transcripts as defined in Table 1 below.

TABLE 1
Target transcripts

| | |
|---|---|
| Target gene | Transcript sequence (NCBI, ENSEMBL) |

| | | |
|---|---|---|
| B2M | NM_004048 | ENST00000648006 |
| | | ENST00000559916 |
| CIITA | NM_000246 | ENST00000324288 |
| | NM_001286402 | ENST00000618327 |
| | NM_001286403 | ENST00000381835 |
| | NM_001379330 | ENST00000646979 |
| | NM_001379331 | ENST00000637439 |
| | NM_001379332 | ENST00000576601 |
| | NM_001379333 | ENST00000636238 |
| | NM_001379334 | ENST00000324288 |
| | | ENST00000618327 |
| | | ENST00000381835 |
| | | ENST00000646979 |
| | | ENST00000637439 |
| | | ENST00000576601 |
| | | ENST00000636238 |
| NLRC5 | NM_001384950 | ENST00000688547 |
| | NM_001330552 | ENST00000262510 |
| | NM_001384951 | ENST00000539144 |
| | NM_001384952 | ENST00000538805 |
| | NM_001384953 | ENST00000538110 |
| | NM_001384954 | ENST00000543030 |
| | NM_001384955 | ENST00000544641 |
| | NM_001384956 | ENST00000399221 |
| | NM_001384957 | ENST00000538059 |
| | NM_001384958 | |
| | NM_001384959 | |
| | NM_001384960 | |
| | NM_001384961 | |
| | NM_001384962 | |
| | NM_001384963 | |
| | NM_001384964 | |
| | NM_001384966 | |
| | NM_001384967 | |
| | NM_001384969 | |
| | NM_001384970 | |
| | NM_001384971 | |
| | NM_032206 | |
| CD3z | NM_198053 | ENST00000362089 |
| | NM_001378515 | ENST00000392122 |
| | NM_001378516 | |
| TRAC | N/A | ENST00000611116 |

In Table 1, the first column indicates the target protein/gene in question, and the second and third columns indicate the transcript of that gene which is preferably to be targeted by any miRNA expression construct of the present invention which targets the expression of the gene indicated in the first column. The second column provides a transcript by reference to the NCBI database, and the third column defines a transcript by reference to the ENSEMBL database. These database references correspond to the published main versions of the databases accessible online on 3 May 2023. Thus, it is understood for example that, for any miRNA expression construct of the present invention that includes a miRNA hairpin targeting CD3z, the miRNA hairpin targeting CD3z can preferably target any of the NM_198053, NM_001378515, NM_001378516, ENST00000362089 and ENST00000392122 transcripts. Thus, the hairpin targeting CD3z preferably targets a sequence comprised within one of the listed transcripts. This applies for every gene listed in Table 1 and the associated transcripts. In an embodiment, the miRNA expression construct of the present invention targeting a gene listed in Table 1 targets a transcript with 80%, 81%, 82%, 83%, 84%, 85%, 86%, 87%, 88%, 89%, 90%, 91%, 92%, 93%, 94%, 95%, 96%, 97%, 98%, 99% or 100% identity to a transcript listed in Table 1. The transcript in question is that which corresponds to the gene that is targeted. Preferably with the miRNA expression construct of the present invention, this can be the case for all of the named genes which are targeted, such as all two, three, four, five or six of the named genes which are targeted.

In an embodiment, one or more cell surface-expressed polypeptides involved in immune signalling are up-regulated. It is to be understood that up-regulation merely refers to increasing the expression of the polypeptides, which can be from zero expression, and does not necessarily imply that the polypeptides were previously expressed. For example, up-regulation in respect of CAR, a polypeptide that is not naturally expressed, may involve expressing CAR. For example, up-regulation of CD47 and/or PD-L1, if one or more of these polypeptides is already expressed in a donor cell, may involve increasing the expression of CD47 and/or PD-L1. In an embodiment, up-regulation comprises expressing the polypeptides. In an embodiment, the polypeptides were not expressed by the equivalent unmodified donor cell.

In an embodiment, the up-regulation is achieved by an expressed transcript. In an embodiment, the expressed transcript encodes one or more of the cell surface-expressed polypeptides involved in immune signalling. In an embodiment, the expressed transcript is a sequence encoding a protein. In an embodiment, the expressed transcript is a nucleic acid. In an embodiment, the expressed transcript is a non-endogenous nucleic acid. In an embodiment, the expressed transcript comprises RNA. In an embodiment, the expressed transcript comprises mRNA encoding the polypeptide.

In an embodiment, the up-regulated surface-expressed polypeptide involved in immune signalling is selected from the group consisting of a non-classical HLA class I polypeptide, CD47, PD-L1 and a CAR.

In an embodiment, the up-regulated surface-expressed polypeptide involved in immune signalling is a non-classical HLA-I polypeptide. In an embodiment, the non-classical HLA class I polypeptide is a HLA-E, HLA-G, or HLA-F polypeptide. In an embodiment, the non-classical HLA class I polypeptide is a genetically modified HLA-E, HLA-G, or HLA-F polypeptide. In an embodiment, the genetically modified HLA-E, HLA-G, and/or HLA-F polypeptide is a B2M fusion protein. In an embodiment, the non-classical HLA class I molecule is a HLA-E-B2M fusion protein, HLA-G-B2M fusion protein, or HLA-F-B2M fusion protein. In an embodiment, a B2M fusion protein comprises the non-classical HLA polypeptide in question, or a functional fragment thereof, and a B2M polypeptide, or a functional fragment thereof. In an embodiment, the expression of the HLA-E, HLA-G, and/or HLA-F polypeptide is not inhibited by miRNA of the present invention that targets endogenous HLA I and/or II.

In an embodiment, the non-classical HLA class I molecule is a HLA-B2M fusion protein comprising the B2M polypeptide encoded by SEQ ID NO: 27, or a sequence having at least 80%, 85%, 90%, 95%, 96%, 97%, 98%, 99% or 100% identity thereto. In an embodiment, the non-classical HLA class I molecule is a HLA-E-B2M fusion protein comprising the B2M polypeptide encoded by SEQ ID NO: 27, or a sequence having at least 80%, 85%, 90%, 95%, 96%, 97%, 98%, 99% or 100% identity thereto. In an embodiment, the non-classical HLA class I molecule is a HLA-F-B2M fusion protein comprising the B2M polypeptide encoded by SEQ ID NO: 27, or a sequence having at least 80%, 85%, 90%, 95%, 96%, 97%, 98%, 99% or 100% identity thereto. In an embodiment, the non-classical HLA class I molecule is a HLA-G-B2M fusion protein comprising the B2M polypeptide encoded by SEQ ID NO: 27, or a sequence having at least 80%, 85%, 90%, 95%, 96%, 97%, 98%, 99% or 100% identity thereto. In a preferred embodiment of these types, the up-regulation of the non-classical HLA class I molecule is achieved by a codon-optimised sequence. In a preferred embodiment of these types, the up-regulation of the non-classical HLA class I molecule is achieved by the expression of a sequence comprising SEQ ID NO: 27, or a sequence having at least 80%, 85%, 90%, 95%, 96%, 97%, 98%, 99% or 100% identity thereto. In an embodiment, the sequence is the RNA, such as mRNA, equivalent of SEQ ID NO: 27, or a sequence having at least 80%, 85%, 90%, 95%, 96%, 97%, 98%, 99% or 100% identity thereto.

In an embodiment, the non-classical HLA class I molecule is a HLA-E-B2M fusion protein encoded by SEQ ID NO: 19, or a sequence having at least 80%, 85%, 90%, 95%, 96%, 97%, 98%, 99% or 100% identity thereto. In an embodiment, the non-classical HLA class I molecule is a HLA-E-B2M fusion protein comprising the sequence of SEQ ID NO: 20 or a sequence having at least 80%, 85%, 90%, 95%, 96%, 97%, 98%, 99% or 100% identity thereto.

In a preferred embodiment, the non-classical HLA class I molecule is a HLA-E-B2M fusion protein encoded by a codon-optimised nucleotide sequence. In a preferred embodiment, the non-classical HLA class I molecule is encoded by SEQ ID NO: 21, or a sequence having at least 80%, 85%, 90%, 95%, 96%, 97%, 98%, 99% or 100% identity thereto. In an embodiment, the non-classical HLA class I molecule is a HLA-E-B2M fusion protein comprising the sequence of SEQ ID NO: 22 or a sequence having at least 80%, 85%, 90%, 95%, 96%, 97%, 98%, 99% or 100% identity thereto.

In an embodiment, the up-regulated surface-expressed polypeptide involved in immune signalling is CD47. Herein, CD47 includes full-length and truncated polypeptides such as functional fragments of CD47. In an embodiment, CD47 is up-regulated to inhibit phagocytosis of the engineered donor cells by macrophages. In an embodiment, the polypeptide is a functional fragment of CD47. In an embodiment, the polypeptide is a truncated CD47 polypeptide. In an embodiment, the truncated CD47 polypeptide is encoded by the sequence of SEQ ID NO: 23 or a sequence having at least 80%, 85%, 90%, 95%, 96%, 97%, 98%, 99% or 100% identity thereto. In an embodiment, the truncated CD47 polypeptide comprises the sequence of SEQ ID NO: 24 or a sequence having at least 80%, 85%, 90%, 95%, 96%, 97%, 98%, 99% or 100% identity thereto.

In an embodiment, the up-regulated surface-expressed polypeptide involved in immune signalling is PD-L1. Herein, PD-L1 includes full-length and truncated polypeptides such as functional fragments of PD-L1. In an embodiment, PD-L1 is up-regulated to induce anergy/exhaustion of bystander T-cells. In an embodiment, the polypeptide is a functional fragment of PD-L1. In an embodiment, PD-L1 is encoded by the sequence of SEQ ID NO: 25 or a sequence having at least 80%, 85%, 90%, 95%, 96%, 97%, 98%, 99% or 100% identity thereto. In an embodiment, the truncated PD-L1 polypeptide comprises the sequence of SEQ ID NO: 26 or a sequence having at least 80%, 85%, 90%, 95%, 96%, 97%, 98%, 99% or 100% identity thereto.

In an embodiment, the up-regulated surface-expressed polypeptide involved in immune signalling is a CAR. In an embodiment, the CAR is an anti-CD19 CAR. In an embodiment, the CAR is encoded by SEQ ID NO: 17, or a sequence having at least 80%, 85%, 90%, 95%, 96%, 97%, 98%, 99% or 100% identity thereto. In an embodiment, the CAR comprises the sequence of SEQ ID NO: 18 or a sequence having at least 80%, 85%, 90%, 95%, 96%, 97%, 98%, 99% or 100% identity thereto.

In an embodiment, a surface-expressed non-classical HLA class I is up-regulated and a surface-expressed HLA class I is down-regulated. In an embodiment, a surface-expressed non-classical HLA class I is up-regulated and a surface-expressed classical HLA class I is down-regulated. In an embodiment, a surface-expressed non-classical HLA class I is up-regulated and a surface-expressed endogenous HLA class I is down-regulated. In preferred embodiments of this type, the down-regulated HLA class I is effectively silenced. Thus, in such embodiments, the down-regulated HLA class I inhibits CD8+ T-cell killing, and the up-regulated non-classical HLA class I inhibits NK killing.

In an embodiment, a surface expressed CAR is up-regulated and a surface expressed TCR is down-regulated. In an embodiment, the TCR is down-regulated by miRNA targeting a sequence that is comprised in the endogenous TCR sequence, and that is not comprised in the CAR sequence. In an embodiment, down-regulation of the TCR is achieved by miRNA that does not target the CD3z activation domain. This is particularly preferred in any embodiment of the present invention wherein miRNA inhibits the expression of CD3z.

In an embodiment, the engineered donor cell of the present invention further expresses a safety switch gene or suicide gene. In an embodiment, the safety switch or suicide gene facilitates the inducible depletion of the engineered donor cells. In an embodiment, depletion is induced if the engineered donor cells become tumorigenic and/or if engineered donor cells cause an adverse event such as cytokine storm (CRS).

In an embodiment, the suicide gene or safety switch gene is selected from the group consisting of herpes simplex virus thymidine kinase (HSV-tk), inducible caspase 9 (iCasp9), truncated endothelial growth factor receptor (tEGFR), RQR8, dihydrofolate reductase (DHFR), CD20 or a truncated CD20 (tCD20) and thymidylate synthase (TYMS).

In an embodiment, the engineered donor cell of the present invention further expresses a selection gene. In an embodiment, the selection gene is LNGFR, truncated endothelial growth factor receptor (tEGFR), tCD19, CD20 or a truncated CD20 (tCD20), tCD34 or a derivative thereof.

miRNA Expression Constructs for Creating Allogeneic Cells

The second aspect of the present invention relates to the miRNA expression constructs that are present in and can be used to produce the engineered donor cells of the present invention.

In a second aspect, the present invention provides a miRNA expression construct comprising one or more miRNA hairpins targeting B2M, NLRC5, TAP1, TAP2, TAPBP, RFX5, RFXANK, RFXAP, CIITA, TCRa, TCRb, CD3d, CD3g, CD3e and/or CD3z.

In an embodiment, the construct further comprises an expressed transcript. In an embodiment, the expressed transcript is a sequence encoding a protein.

In an embodiment, the miRNA expression construct comprises at least a first and a second miRNA hairpin, wherein the first miRNA hairpin and the second miRNA hairpin target a combination of two sequences independently selected from sequences having at least 80%, 85%, 90%, 95%, 96%, 97%, 98%, 99% or 100% to any one of SEQ ID NOs: 1 to 16.

In an embodiment, the miRNA expression construct comprises at least a first and a second miRNA hairpin, wherein the first miRNA hairpin and the second miRNA hairpin target a combination selected from SEQ ID NO: 1 and 2, SEQ ID NO: 1 and 3, SEQ ID NO: 1 and 4, SEQ ID NO: 1 and 5, SEQ ID NO: 1 and 6, SEQ ID NO: 1 and 7, SEQ ID NO: 1 and 8, SEQ ID NO: 1 and 9, SEQ ID NO: 1 and 10, SEQ ID NO: 1 and 11, SEQ ID NO: 1 and 12, SEQ ID NO: 1 and 13, SEQ ID NO: 1 and 14, SEQ ID NO: 1 and 15, SEQ ID NO: 1 and 16, SEQ ID NO: 2 and 2, SEQ ID NO: 2 and 3, SEQ ID NO: 2 and 4, SEQ ID NO: 2 and 5, SEQ ID NO: 2 and 6, SEQ ID NO: 2 and 7, SEQ ID NO: 2 and 8, SEQ ID NO: 2 and 9, SEQ ID NO: 2 and 10, SEQ ID NO: 2 and 11, SEQ ID NO: 2 and 12, SEQ ID NO: 2 and 13, SEQ ID NO: 2 and 14, SEQ ID NO: 2 and 15, SEQ ID NO: 2 and 16, SEQ ID NO: 3 and 3, SEQ ID NO: 3 and 4, SEQ ID NO: 3 and 5, SEQ ID NO: 3 and 6, SEQ ID NO: 3 and 7, SEQ ID NO: 3 and 8, SEQ ID NO: 3 and 9, SEQ ID NO: 3 and 10, SEQ ID NO: 3 and 11, SEQ ID NO: 3 and 12, SEQ ID NO: 3 and 13, SEQ ID NO: 3 and 14, SEQ ID NO: 3 and 15, SEQ ID NO: 3 and 16, SEQ ID NO: 4 and 4, SEQ ID NO: 4 and 5, SEQ ID NO: 4 and 6, SEQ ID NO: 4 and 7, SEQ ID NO: 4 and 8, SEQ ID NO: 4 and 9, SEQ ID NO: 4 and 10, SEQ ID NO: 4 and 11, SEQ ID NO: 4 and 12, SEQ ID NO: 4 and 13, SEQ ID NO: 4 and 14, SEQ ID NO: 4 and 15, SEQ ID NO: 4 and 16, SEQ ID NO: 5 and 5, SEQ ID NO: 5 and 6, SEQ ID NO: 5 and 7, SEQ ID NO: 5 and 8, SEQ ID NO: 5 and 9, SEQ ID NO: 5 and 10, SEQ ID NO: 5 and 11, SEQ ID NO: 5 and 12, SEQ ID NO: 5 and 13, SEQ ID NO: 5 and 14, SEQ ID NO: 5 and 15, SEQ ID NO: 5 and 16, SEQ ID NO: 6 and 6, SEQ ID NO: 6 and 7, SEQ ID NO: 6 and 8, SEQ ID NO: 6 and 9, SEQ ID NO: 6 and 10, SEQ ID NO: 6 and 11, SEQ ID NO: 6 and 12, SEQ ID NO: 6 and 13, SEQ ID NO: 6 and 14, SEQ ID NO: 6 and 15, SEQ ID NO: 6 and 16, SEQ ID NO: 7 and 7, SEQ ID NO: 7 and 8, SEQ ID NO: 7 and 9, SEQ ID NO: 7 and 10, SEQ ID NO: 7 and 11, SEQ ID NO: 7 and 12, SEQ ID NO: 7 and 13, SEQ ID NO: 7 and 14, SEQ ID NO: 7 and 15, SEQ ID NO: 7 and 16, SEQ ID NO: 8 and 8, SEQ ID NO: 8 and 9, SEQ ID NO: 8 and 10, SEQ ID NO: 8 and 11, SEQ ID NO: 8 and 12, SEQ ID NO: 8 and 13, SEQ ID NO: 8 and 14, SEQ ID NO: 8 and 15, SEQ ID NO: 8 and 16, SEQ ID NO: 9 and 9, SEQ ID NO: 9 and 10, SEQ ID NO: 9 and 11, SEQ ID NO: 9 and 12, SEQ ID NO: 9 and 13, SEQ ID NO: 9 and 14, SEQ ID NO: 9 and 15, SEQ ID NO: 9 and 16, SEQ ID NO: 10 and 10, SEQ ID NO: 10 and 11, SEQ ID NO: 10 and 12, SEQ ID NO: 10 and 13, SEQ ID NO: 10 and 14, SEQ ID NO: 10 and 15, SEQ ID NO: 10 and 16, SEQ ID NO: 11 and 11, SEQ ID NO: 11 and 12, SEQ ID NO: 11 and 13, SEQ ID NO: 11 and 14, SEQ ID NO: 11 and 15, SEQ ID NO: 11 and 16, SEQ ID NO: 12 and 12, SEQ ID NO: 12 and 13, SEQ ID NO: 12 and 14, SEQ ID NO: 12 and 15, SEQ ID NO: 12 and 16, SEQ ID NO: 13 and 13, SEQ ID NO: 13 and 14, SEQ ID NO: 13 and 15, SEQ ID NO: 13 and 16, SEQ ID NO: 14 and 14, SEQ ID NO: 14 and 15, SEQ ID NO: 14 and 16, SEQ ID NO: 15 and 15, SEQ ID NO: 15 and 16, and SEQ ID NO: 16 and 16 respectively.

In an embodiment, the miRNA expression construct comprises at least a first and a second miRNA hairpin, wherein the first miRNA hairpin and the second miRNA hairpin target a combination selected from SEQ ID NO: 30 and 31, SEQ ID NO: 30 and 32, SEQ ID NO: 30 and 33, SEQ ID NO: 30 and 34, SEQ ID NO: 30 and 35, SEQ ID NO: 30 and 36, SEQ ID NO: 30 and 37, SEQ ID NO: 30 and 38, SEQ ID NO: 30 and 39, SEQ ID NO: 30 and 40, SEQ ID NO: 30 and 41, SEQ ID NO: 30 and 42, SEQ ID NO: 30 and 43, SEQ ID NO: 30 and 44, SEQ ID NO: 31 and 31, SEQ ID NO: 31 and 32, SEQ ID NO: 31 and 33, SEQ ID NO: 31 and 34, SEQ ID NO: 31 and 35, SEQ ID NO: 31 and 36, SEQ ID NO: 31 and 37, SEQ ID NO: 31 and 38, SEQ ID NO: 31 and 39, SEQ ID NO: 31 and 40, SEQ ID NO: 31 and 41, SEQ ID NO: 31 and 42, SEQ ID NO: 31 and 43, SEQ ID NO: 32 and 32, SEQ ID NO: 32 and 33, SEQ ID NO: 32 and 34, SEQ ID NO: 32 and 35, SEQ ID NO: 32 and 36, SEQ ID NO: 32 and 37, SEQ ID NO: 32 and 38, SEQ ID NO: 32 and 39, SEQ ID NO: 32 and 40, SEQ ID NO: 32 and 41, SEQ ID NO: 32 and 42, SEQ ID NO: 32 and 43, SEQ ID NO: 33 and 33, SEQ ID NO: 33 and 34, SEQ ID NO: 33 and 35, SEQ ID NO: 33 and 36, SEQ ID NO: 33 and 37, SEQ ID NO: 33 and 38, SEQ ID NO: 33 and 39, SEQ ID NO: 33 and 40, SEQ ID NO: 33 and 41, SEQ ID NO: 33 and 42, SEQ ID NO: 33 and 43, SEQ ID NO: 34 and 34, SEQ ID NO: 34 and 35, SEQ ID NO: 34 and 36, SEQ ID NO: 34 and 37, SEQ ID NO: 34 and 38, SEQ ID NO: 34 and 39, SEQ ID NO: 34 and 40, SEQ ID NO: 34 and 41, SEQ ID NO: 34 and 42, SEQ ID NO: 34 and 43, SEQ ID NO: 35 and 35, SEQ ID NO: 35 and 36, SEQ ID NO: 35 and 37, SEQ ID NO: 35 and 38, SEQ ID NO: 35 and 39, SEQ ID NO: 35 and 40, SEQ ID NO: 35 and 41, SEQ ID NO: 35 and 42, SEQ ID NO: 35 and 43, SEQ ID NO: 36 and 36, SEQ ID NO: 36 and 37, SEQ ID NO: 36 and 38, SEQ ID NO: 36 and 39, SEQ ID NO: 36 and 40, SEQ ID NO: 36 and 41, SEQ ID NO: 36 and 42, SEQ ID NO: 36 and 43, SEQ ID NO: 37 and 37, SEQ ID NO: 37 and 38, SEQ ID NO: 37 and 39, SEQ ID NO: 37 and 40, SEQ ID NO: 37 and 41, SEQ ID NO: 37 and 42, SEQ ID NO: 37 and 43, SEQ ID NO: 38 and 38, SEQ ID NO: 38 and 39, SEQ ID NO: 38 and 40, SEQ ID NO: 38 and 41, SEQ ID NO: 38 and 42, SEQ ID NO: 38 and 43, SEQ ID NO: 39 and 39, SEQ ID NO: 39 and 40, SEQ ID NO: 39 and 41, SEQ ID NO: 39 and 42, SEQ ID NO: 39 and 43, SEQ ID NO: 40 and 40, SEQ ID NO: 40 and 41, SEQ ID NO: 40 and 42, SEQ ID NO: 40 and 43, SEQ ID NO: 41 and 41, SEQ ID NO: 41 and 42, SEQ ID NO: 41 and 43, SEQ ID NO: 42 and 42, SEQ ID NO: 42 and 43 and SEQ ID NO: 43 and 43 respectively.

In an embodiment, the miRNA expression construct comprises at least a first and a second miRNA hairpin, wherein the first miRNA hairpin and the second miRNA hairpin target a combination selected from SEQ ID NO: 30 and 44, SEQ ID NO: 30 and 45, SEQ ID NO: 30 and 45, SEQ ID NO: 30 and 46, SEQ ID NO: 30 and 47, SEQ ID NO: 30 and 48, SEQ ID NO: 30 and 49, SEQ ID NO: 30 and 50, SEQ ID NO: 30 and 51, SEQ ID NO: 30 and 52, SEQ ID NO: 30 and 53, SEQ ID NO: 30 and 54, SEQ ID NO: 30 and 55, SEQ ID NO: 30 and 56, SEQ ID NO: 30 and 57, SEQ ID NO: 31 and 44, SEQ ID NO: 31 and 45, SEQ ID NO: 31 and 46, SEQ ID NO: 31 and 47, SEQ ID NO: 31 and 48, SEQ ID NO: 31 and 49, SEQ ID NO: 31 and 50, SEQ ID NO: 31 and 51, SEQ ID NO: 31 and 52, SEQ ID NO: 31 and 53, SEQ ID NO: 31 and 54, SEQ ID NO: 31 and 55, SEQ ID NO: 31 and 56, SEQ ID NO: 31 and 57, SEQ ID NO: 44 and 44, SEQ ID NO: 44 and 45, SEQ ID NO: 44 and 46, SEQ ID NO: 44 and 47, SEQ ID NO: 44 and 48, SEQ ID NO: 44 and 49, SEQ ID NO: 44 and 50, SEQ ID NO: 44 and 51, SEQ ID NO: 44 and 52, SEQ ID NO: 44 and 53, SEQ ID NO: 44 and 54, SEQ ID NO: 44 and 55, SEQ ID NO: 44 and 56, SEQ ID NO: 44 and 57, SEQ ID NO: 45 and 45, SEQ ID NO: 45 and 46, SEQ ID NO: 45 and 47, SEQ ID NO: 45 and 48, SEQ ID NO: 45 and 49, SEQ ID NO: 45 and 50, SEQ ID NO: 45 and 51, SEQ ID NO: 45 and 52, SEQ ID NO: 45 and 53, SEQ ID NO: 45 and 54, SEQ ID NO: 45 and 55, SEQ ID NO: 45 and 56, SEQ ID NO: 45 and 57, SEQ ID NO: 46 and 46, SEQ ID NO: 46 and 47, SEQ ID NO: 46 and 48, SEQ ID NO: 46 and 49, SEQ ID NO: 46 and 50, SEQ ID NO: 46 and 51, SEQ ID NO: 46 and 52, SEQ ID NO: 46 and 53, SEQ ID NO: 46 and 54, SEQ ID NO: 46 and 55, SEQ ID NO: 46 and 56, SEQ ID NO: 46 and 57, SEQ ID NO: 47 and 47, SEQ ID NO: 47 and 48, SEQ ID NO: 47 and 49, SEQ ID NO: 47 and 50, SEQ ID NO: 47 and 51, SEQ ID NO: 47 and 52, SEQ ID NO: 47 and 53, SEQ ID NO: 47 and 54, SEQ ID NO: 47 and 55, SEQ ID NO: 47 and 56, SEQ ID NO: 47 and 57, SEQ ID NO: 48 and 48, SEQ ID NO: 48 and 49, SEQ ID NO: 48 and 50, SEQ ID NO: 48 and 51, SEQ ID NO: 48 and 52, SEQ ID NO: 48 and 53, SEQ ID NO: 48 and 54, SEQ ID NO: 48 and 55, SEQ ID NO: 48 and 56, SEQ ID NO: 48 and 57, SEQ ID NO: 49 and 49, SEQ ID NO: 49 and 50, SEQ ID NO: 49 and 51, SEQ ID NO: 49 and 52, SEQ ID NO: 49 and 53, SEQ ID NO: 49 and 54, SEQ ID NO: 49 and 55, SEQ ID NO: 49 and 56, SEQ ID NO: 49 and 57, SEQ ID NO: 50 and 50, SEQ ID NO: 50 and 51, SEQ ID NO: 50 and 52, SEQ ID NO: 50 and 53, SEQ ID NO: 50 and 54, SEQ ID NO: 50 and 55, SEQ ID NO: 50 and 56, SEQ ID NO: 50 and 57, SEQ ID NO: 51 and 51, SEQ ID NO: 51 and 52, SEQ ID NO: 51 and 53, SEQ ID NO: 51 and 54, SEQ ID NO: 51 and 55, SEQ ID NO: 51 and 56, SEQ ID NO: 51 and 57, SEQ ID NO: 52 and 52, SEQ ID NO: 52 and 53, SEQ ID NO: 52 and 54, SEQ ID NO: 52 and 55, SEQ ID NO: 52 and 56, SEQ ID NO: 52 and 57, SEQ ID NO: 53 and 53, SEQ ID NO: 53 and 54, SEQ ID NO: 53 and 55, SEQ ID NO: 53 and 56, SEQ ID NO: 53 and 57, SEQ ID NO: 54 and 54, SEQ ID NO: 54 and 55, SEQ ID NO: 54 and 56, SEQ ID NO: 54 and 57, SEQ ID NO: 55 and 55, SEQ ID NO: 55 and 56, SEQ ID NO: 55 and 57, SEQ ID NO: 56 and 56, SEQ ID NO: 56 and 57 and SEQ ID NO: 57 and 57.

The terms “first” and “second” etc. miRNA hairpin herein are understood as not necessarily referring to the genetic order of the miRNA hairpin, but are merely used to define a number of separate miRNA hairpin elements.

In an embodiment, there are two copies of the first miRNA hairpin and/or two copies of the second miRNA hairpin.

In an embodiment, there are three copies of the first miRNA hairpin and/or three copies of the second miRNA hairpin.

In an embodiment, the miRNA expression construct comprises at least two different miRNA hairpins which target different regions of the same transcript.

In an embodiment, the miRNA expression construct comprises at least two different miRNA hairpins which target different transcripts of the same gene.

In an embodiment, the miRNA expression construct comprises at least two different miRNA hairpins which target different splice variants of the same gene.

In an embodiment, the miRNA expression construct further comprises a promoter element. In an embodiment, the promoter element is a promoter. In an embodiment, the promoter is a eukaryotic promoter. In an embodiment, the eukaryotic promoter is a Pol II or Pol III promoter. In an embodiment, the promoter is an inducible promoter, a tissue-specific promoter, a cell lineage-specific promoter or a synthetic promoter. In an embodiment, the promoter element is selected from the promoter elements of Table 2. In an embodiment, the promoter is a UBI promoter. In an embodiment, the promoter is an EF1α promoter, a derivative of an EF1α promoter or an EF1 short promoter.

In an embodiment, the miRNA expression construct further comprises a spacer. In an embodiment, the spacer comprises an enhancer. In an embodiment, the spacer is an enhancer. In an embodiment, the spacer is at least 50 nucleotides in length. In an embodiment, the spacer is between 50 and 1,000 nucleotides in length. In an embodiment, the spacer is between 50 and 900, 50 and 800, 100 and 800, or 50 and 800 nucleotides in length. In an embodiment, the spacer is at least 60, 70, 80, 90, 100, 110, 120, 130, 140, 150, 160, 170, 180 or 200 nucleotides in length. In an embodiment, the spacer is a GFP sequence. In a preferred embodiment, the spacer is a GFP1 sequence. In a most preferred embodiment, the spacer is a GFP1 sequence encoded by a sequence comprising SEQ ID NO: 28 or a sequence having 80%, 85%, 90%, 95%, 96%, 97%, 98%, 99% or 100% identity thereto. In a preferred embodiment, the spacer is a GFP2 sequence. In a most preferred embodiment, the spacer is a GFP2 sequence encoded by a sequence comprising SEQ ID NO: 29 or a sequence having 80%, 85%, 90%, 95%, 96%, 97%, 98%, 99% or 100% identity thereto.

In an embodiment, the spacer is positioned between the promoter and the miRNA hairpins. In an embodiment, the spacer is heterologous with respect to the promoter element. In an embodiment, the spacer comprises an encoded open reading frame.

In an embodiment, at least two of the miRNA hairpins are separated by an intervening sequence.

In an embodiment, the expressed transcript is at least one gene selected from the group consisting of non-classical HLA class I, Chimeric antigen receptors, CD47 and PD-L1.

In an embodiment, expressed transcript comprises a sequence having at least 80%, 85%, 90%, 95%, 96%, 97%, 98%, 99% or 100% sequence identity to SEQ ID NO: 17, 19 or 21. In an embodiment, the expressed transcript encodes a protein having at least 80%, 85%, 90%, 95%, 96%, 97%, 98%, 99% or 100% sequence identity to SEQ ID NO: 18, 20 or 22.

In an embodiment, the expressed transcript encodes any non-classical HLA as defined in respect of the up-regulated non-classical HLA that is comprised in the engineered donor cell of the present invention.

In an embodiment, the miRNA expression construct comprises a sequence encoding a moiety for re-directing immune effector cell function. In an embodiment, the miRNA expression construct comprises a sequence encoding an engineered T-cell receptor. In an embodiment, the miRNA expression construct comprises a sequence encoding a CAR. In an embodiment, the CAR is a CAR that targets HIV infected cells or tumor cells, optionally wherein the CAR is an anti-CD19 CAR, optionally wherein the CAR is FMC63. In an embodiment, the chimeric antigen receptor is a bispecific chimeric antigen receptor or a dual chimeric antigen receptor.

In an embodiment, the miRNA hairpins of the miRNA expression construct are under the control of a first promoter and the sequence encoding the T-cell receptor or chimeric antigen receptor is under the control of a second promoter, or wherein the miRNA hairpins and the sequence encoding the T-cell receptor or chimeric antigen receptor are under the control of a single promoter. In an embodiment, the miRNA hairpins are under the control of a first promoter and the sequence encoding the chimeric antigen receptor is under the control of a second promoter. In another embodiment, the miRNA hairpins and chimeric antigen receptor are under the control of the same promoter.

In an embodiment, miRNA expression construct further comprises a T cell receptor sequence.

In an embodiment, the miRNA expression construct further comprises a selection gene. In an embodiment, the selection gene is LNGFR, truncated endothelial growth factor receptor (tEGFR), tCD19, CD20 or a truncated CD20 (tCD20), tCD34 or a derivative thereof.

In an embodiment, the construct further comprises a sequence encoding a suicide gene or safety switch gene. In an embodiment, the suicide gene or safety switch gene is selected from the group consisting of herpes simplex virus thymidine kinase (HSV-tk), inducible caspase 9 (iCasp9), truncated endothelial growth factor receptor (tEGFR), RQR8, dihydrofolate reductase (DHFR), CD20 or a truncated CD20 (tCD20) and thymidylate synthase (TYMS).

In an embodiment, the miRNA expression construct further comprises an internal ribosome entry site (IRES). In an embodiment, the construct further comprises a peptide cleavage site. In an embodiment, the peptide cleavage site is a 2A peptide. In an embodiment, the 2A peptide is selected from the group comprising: 2A, P2A, T2A, E2A, F2A, BmCPV 2A, and BmIFV 2A.

In an embodiment, the miRNA expression construct is an isolated nucleic acid.

In an embodiment, the first nucleotide in the miRNA target sequence in one or more or all of the hairpins in the miRNA expression construct is a thymidine nucleotide.

In a particularly preferred embodiment, the miRNA expression construct comprises an EF1s promoter and a spacer comprising an enhancer.

Specific miRNA Expression Constructs

In an embodiment, the miRNA expression construct of the present invention comprises a single miRNA hairpin targeting B2M. In an embodiment, the miRNA expression construct of the present invention comprises two miRNA hairpins targeting B2M. In an embodiment, the miRNA expression construct of the present invention comprises three miRNA hairpins targeting B2M.

In an embodiment, the miRNA expression construct of the present invention comprises a single miRNA hairpin targeting TRAC. In an embodiment, the miRNA expression construct of the present invention comprises two miRNA hairpins targeting TRAC. In an embodiment, the miRNA expression construct of the present invention comprises three miRNA hairpins targeting TRAC.

In an embodiment, the miRNA expression construct of the present invention that targets TRAC further comprises miRNA hairpins targeting one or more other TCR complex polypeptides. In an embodiment, the miRNA expression construct of the present invention that targets TRAC further comprises one or more miRNA hairpins targeting TCRB, CD3d, CD3g, CD3e and/or CD3z. In an embodiment, the miRNA expression construct of the present invention that targets TRAC further comprises one or more miRNA hairpins targeting CD3z. In an embodiment, the miRNA expression construct of the present invention that targets TRAC further comprises a single miRNA hairpin targeting CD3z.

In an embodiment, the miRNA expression construct of the present invention comprises a single miRNA hairpin targeting CD3z. In an embodiment, the miRNA expression construct of the present invention comprises two miRNA hairpins targeting CD3z. In an embodiment, the miRNA expression construct of the present invention comprises three miRNA hairpins targeting CD3z.

In an embodiment, the miRNA expression construct of the present invention comprises a first miRNA hairpin targeting a first CD3z transcript region and a second miRNA hairpin targeting a second different CD3z transcript region. In an embodiment, the miRNA expression construct of the present invention comprises no more than two miRNA hairpins targeting CD3z, a first miRNA hairpin targeting a first CD3z transcript region and a second miRNA hairpin targeting a second different CD3z transcript region. In an embodiment, the miRNA expression construct targeting more than one different CD3z transcript region provides increased silencing over the equivalent miRNA expression construct comprising miRNA hairpins, such as two miRNA hairpins, that only target the same CD3z transcript sequence.

In an embodiment, the miRNA expression construct of the present invention comprises a single miRNA hairpin targeting CIITA. In an embodiment, the miRNA expression construct of the present invention comprises two miRNA hairpins targeting CIITA. In an embodiment, the miRNA expression construct of the present invention comprises three miRNA hairpins targeting CIITA.

In an embodiment, the miRNA expression construct of the present invention comprises a first miRNA hairpin targeting a first CD3z transcript sequence and a second miRNA hairpin targeting a second, different CD3z transcript sequence. In an embodiment, the miRNA expression construct further comprises a third miRNA hairpin targeting a B2M transcript sequence. In an embodiment, the miRNA expression construct further comprises a fourth miRNA hairpin targeting the same B2M transcript sequence. In an embodiment, the miRNA expression construct further comprises a sequence encoding a CAR. In an embodiment, the CAR is an anti-CD19 CAR. In an embodiment, the CAR is encoded by SEQ ID NO: 17, or a sequence having at least 80%, 85%, 90%, 95%, 96%, 97%, 98%, 99% or 100% identity thereto. In an embodiment, the CAR comprises the sequence of SEQ ID NO: 18 or a sequence having at least 80%, 85%, 90%, 95%, 96%, 97%, 98%, 99% or 100% identity thereto. In an embodiment, the CAR is expressed from a PGK promoter.

In an embodiment, the miRNA expression construct of the present invention comprises a first miRNA hairpin targeting CD3z_T1 or a sequence having at least 80%, 85%, 90%, 95%, 96%, 97%, 98%, 99% or 100% identity thereto and a second miRNA hairpin targeting CD3z_T2 or a sequence having at least 80%, 85%, 90%, 95%, 96%, 97%, 98%, 99% or 100% identity thereto. In a first embodiment, the miRNA expression construct further comprises a third miRNA hairpin targeting B2M_T2 or a sequence having at least 80%, 85%, 90%, 95%, 96%, 97%, 98%, 99% or 100% identity thereto, or a third miRNA hairpin targeting B2M_T5 or a sequence having at least 80%, 85%, 90%, 95%, 96%, 97%, 98%, 99% or 100% identity thereto. In a second, different embodiment, the miRNA expression construct comprises a third and fourth miRNA hairpin each targeting B2M_T5 or the same sequence having at least 80%, 85%, 90%, 95%, 96%, 97%, 98%, 99% or 100% identity thereto. In an embodiment, the miRNA expression construct further comprises a sequence encoding a CAR. In an embodiment, the CAR is an anti-CD19 CAR. In an embodiment, the CAR is encoded by SEQ ID NO: 17, or a sequence having at least 80%, 85%, 90%, 95%, 96%, 97%, 98%, 99% or 100% identity thereto. In an embodiment, the CAR comprises the sequence of SEQ ID NO: 18 or a sequence having at least 80%, 85%, 90%, 95%, 96%, 97%, 98%, 99% or 100% identity thereto. In an embodiment, the CAR is expressed from a PGK promoter.

### DNA, Plasmids, Vectors and Associated Cells

In a third aspect, the present invention provides a DNA molecule comprising the miRNA expression construct of the invention.

In a fourth aspect, the present invention provides a plasmid comprising the miRNA expression construct or DNA molecule of the invention.

In a fifth aspect, the present invention provides a vector comprising the miRNA expression construct, DNA molecule or plasmid of the invention.

In an embodiment, the vector is an expression vector. In an embodiment, the expression vector is an adenovirus, an adeno-associated virus, a retrovirus or a lentivirus vector. In an embodiment, the expression vector of the present invention further comprises at least one drug resistance marker.

In an embodiment, the DNA, plasmid or vector of the invention is isolated.

In a sixth aspect, the present invention provides an engineered donor cell comprising the miRNA expression construct, DNA molecule, plasmid or vector of the invention.

### Methods of Manufacture

In an seventh aspect, the present invention provides a method for down-regulating a polypeptide in a cell comprising expressing the miRNA expression construct, DNA molecule, plasmid or vector of the invention in the cell.

In an embodiment, the present invention provides a method for down-regulating HLA-I by providing a miRNA expression construct that inhibits B2M expression and expressing the miRNA expression construct in a cell, wherein the level of HLA-I expression is reduced by between about 50% and about 90% inclusive, wherein the reduction in the level of HLA-I expression is pre-determined by:
a) providing one, two or three miRNA hairpins targeting B2M in the miRNA expression construct;
b) providing B2M hairpins that target different or the same regions of the B2M transcript in the miRNA expression construct; and
c) providing a transduction efficiency of the miRNA expression construct that results in a copy number of one, two or three miRNA expression constructs in the cell.

In an eighth aspect, the present invention provides a method for preparing an engineered donor cell comprising transfecting or transducing a cell with the miRNA expression construct, DNA molecule, plasmid or vector of the invention.

In a ninth aspect, the present invention provides a method for preparing an engineered donor cell from a patient donor or healthy donor comprising:
(a) collecting a cell from the patient; and
(b) transfecting or transducing the cell with the miRNA expression construct, DNA molecule, plasmid or vector of the invention; and
(c) expressing the miRNA expression construct.

In an embodiment, the engineered donor cell is a T-cell. In an embodiment, the miRNA expression construct, DNA molecule, plasmid or vector down-regulates a TCR polypeptide and up-regulates a CAR polypeptide, wherein the engineered donor cell is a CAR T-cell.

In an embodiment, the chimeric antigen receptor targets HIV infected cells or tumour cells, optionally wherein the chimeric antigen receptor is an anti-CD19 chimeric antigen receptor, optionally wherein the chimeric antigen receptor is FMC63.

In an embodiment, the above methods are in vitro or ex vivo methods.

In a tenth aspect, the present invention provides an engineered effector cell obtainable or obtained by the method of the invention.

### Specific Forms of Engineered Donor Cell

Specific forms of the engineered donor cell of the invention are outlined herein.

In an embodiment, the engineered donor cell is a eukaryotic cell. In an embodiment, the engineered donor cell is a mammalian cell.

In an embodiment, the engineered donor cell is an immune effector cell. In an embodiment, immune effector cell is selected from the group comprising: alpha-beta T-cells, gamma-delta T-cells, tumour infiltrating lymphocytes (TILS), TCR-engineered T-cells, CAR T-cells, NK cells, NK/T-cells, T regulatory cells, monocytes and macrophages. In an embodiment, immune effector cell is a CAR T-cell. In particular, in an embodiment wherein the expression of one or more TCR polypeptides is down-regulated and the expression of a CAR is up-regulated, the engineered donor cell is a CAR T-cell.

In an embodiment, the engineered donor cell is a stem cell or a progenitor cell.

In an embodiment, the engineered donor cell is a pluripotent stem cell, such as an embryonic or an induced pluripotent stem cell.

In an embodiment, the engineered donor cell is a multipotent stem cell, such as a haematopoietic stem cell, mesenchymal stem cell, neural stem cell or a muscle stem cell (satellite cell).

In an embodiment, the stem cell is not a human embryonic stem cell. In an embodiment, the stem cell is obtainable without the destruction of human embryonic stem cells. In an embodiment, the stem cell is not an animal embryonic stem cell. In an embodiment, the stem cell is obtainable without the destruction of animal embryonic stem cells.

In an embodiment, the engineered donor cell is a differentiated cell. In an embodiment, the engineered donor cell is a transplant cell. In an embodiment, the engineered donor cell is for use in transplantation or cell therapy applications. In an embodiment, the engineered donor cell is the cell of a transplant tissue or organ that has been excised from a donor. In an embodiment, the engineered donor cell is the cell of an ex vivo tissue or organ. In an embodiment, the engineered donor cell is a pancreatic cell, optionally a pancreatic islet cell or a pancreatic β cell.

In an embodiment, the engineered donor cell is an in vitro cell. In an embodiment, the engineered donor cell is an ex vivo cell. In an embodiment, the engineered donor cell is an isolated engineered donor cell. In an embodiment, the engineered donor cell is not found in nature. In an embodiment, the engineered donor cell comprises non-endogenous miRNA. In an embodiment, the engineered donor cell comprises synthetic miRNA. In an embodiment, the engineered donor cell comprises miRNA produced via recombinant techniques.

### Methods, Uses and Therapies

In an eleventh aspect, the present invention provides a composition comprising the engineered donor cell of the invention.

In a twelfth aspect, the present invention provides the engineered donor cell, miRNA expression construct, DNA molecule, plasmid, vector or composition of the invention, for use in therapy.

In a thirteenth aspect, the present invention provides the engineered donor cell, miRNA expression construct, DNA molecule, plasmid, vector or composition of the invention, for use in a method of treating cancer, an infectious disease, an auto-immune disease or an inherited disorder.

In a fourteenth aspect, the present invention provides a method of treating cancer, an infectious disease, an auto-immune disease or an inherited disorder, comprising administering the engineered donor cell, miRNA expression construct, DNA molecule, plasmid, vector or composition of the invention.

In a fifteenth aspect, the present invention provides the engineered donor cell, miRNA expression construct, DNA molecule, plasmid, vector or composition of the invention, for use in the manufacture a medicament for the treatment of cancer, an infectious disease, an auto-immune disease or an inherited disorder.

In a sixteenth aspect, the present invention provides the engineered donor cell, miRNA expression construct, DNA molecule, plasmid, vector or composition of the invention, for use in a method of stem cell therapy.

In a seventeenth aspect, the present invention provides a method of stem cell therapy, comprising administering the engineered donor cell, miRNA expression construct, DNA molecule, plasmid, vector or composition of the invention.

In a eighteenth aspect, the present invention provides the engineered donor cell, miRNA expression construct, DNA molecule, plasmid, vector or composition of the invention, for use in the manufacture a medicament for stem cell therapy.

### RNA Inhibition

An inhibitory nucleic acid may inhibit the transcript of a gene or prevent the translation of a gene transcript in a cell. An inhibitory nucleic acid may be from 16 to 1000 nucleotides long, and in certain embodiments from 18 to 100 nucleotides long. In certain embodiments, the inhibitory nucleic acid is an isolated nucleic acid that binds or hybridizes to a gene of interest. Inhibitory nucleic acids are well known in the art. For example, siRNA, shRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Since the discovery of RNAi by Fire and colleagues in 1998, the biochemical mechanisms have been rapidly characterized. Double stranded RNA (dsRNA) is cleaved by Dicer, which is an RNAase III family ribonuclease. This process yields miRNAs of ~21 nucleotides in length. These miRNAs are incorporated into a multiprotein RNA-induced silencing complex (RISC) that is guided to target mRNA. RISC cleaves the target mRNA in the middle of the complementary region. In mammalian cells, the related miRNAs are found that are short RNA fragments (~22 nucleotides). miRNAs are generated after Dicer-mediated cleavage of longer (~70 nucleotide) precursors with imperfect hairpin RNA structures. The miRNA is incorporated into a miRNA-protein complex (miRNP), which leads to translational repression of target mRNA.

In designing RNAi there are several factors that may be considered such as the nature of the siRNA, the durability of the silencing effect, and the choice of delivery system. To produce an RNAi effect, the miRNA that is introduced into the organism may typically contain exonic sequences. Furthermore, the RNAi process is homology dependent, so the sequences are often carefully selected so as to maximize gene specificity, while minimizing the possibility of cross-interference between homologous, but not gene-specific sequences. Particularly, the miRNA often exhibits greater than 80, 85, 90, 95, 98% or even 100% identity between the sequence of the miRNA and a portion of the nucleotide sequence of a target gene. Sequences less than about 80% identical to the target gene may be substantially less effective. Thus, the greater identity between the miRNA and the target gene to be inhibited, the less likely expression of unrelated genes will be affected.

In addition, the size of the miRNA is an important consideration. In some embodiments, the present invention relates to miRNA molecules that include at least about 19-25 nucleotides, and are able to modulate target gene expression. In the context of the present invention, the miRNA is particularly less than 500, 200, 100, 50, 25, 24, 23 or 22 nucleotides in length. In some embodiments, the miRNA is from about 25 nucleotides to about 35 nucleotides or from about 19 nucleotides to about 25 nucleotides in length.

To improve the effectiveness of miRNA-mediated gene silencing, guidelines for selection of target sites on mRNA have been developed for optimal design of miRNA (Soutschek et al., 2004; Wadhwa et al., 2004). These strategies may allow for rational approaches for selecting siRNA sequences to achieve maximal gene knockdown. To facilitate the entry of miRNA into cells and tissues, a variety of vectors including plasmids and viral vectors such as adenovirus, lentivirus, and retrovirus have been used (Wadhwa et al., 2004).

Within an inhibitory nucleic acid, the components of a nucleic acid need not be of the same type or homogenous throughout (e.g., an inhibitory nucleic acid may comprise a nucleotide and a nucleic acid or nucleotide analog). Typically, an inhibitory nucleic acid forms a double-stranded structure; the double-stranded structure may result from two separate nucleic acids that are partially or completely complementary. In certain embodiments of the present invention, the inhibitory nucleic acid may comprise only a single nucleic acid (polynucleotide) or nucleic acid analog and form a double-stranded structure by complementing with itself (e.g., forming a hairpin loop). The double-stranded structure of the inhibitory nucleic acid may comprise 16-500 or more contiguous nucleobases, including all ranges therebetween. The inhibitory nucleic acid may comprise 17 to 35 contiguous nucleobases, more particularly 18 to 30 contiguous nucleobases, more particularly 19 to 25 nucleobases, more particularly 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

miRNA can be obtained from commercial sources, natural sources, or can be synthesized using any of a number of techniques well-known to those of ordinary skill in the art. For example, commercial sources of predesigned miRNA include Invitrogen's Stealth Select technology (Carlsbad, CA), Ambion (Austin, TX), and Qiagen (Valencia, CA). An inhibitory nucleic acid that can be applied in the compositions and methods of the present invention may be any nucleic acid sequence that has been found by any source to be a validated down-regulator of a target gene.

In some embodiments, the miRNA molecule is at least 75, 80, 85, or 90% homologous, particularly at least 95%, 99%, or 100% similar or identical, or any percentages in between the foregoing (e.g., the invention contemplates 75% and greater, 80% and greater, 85% and greater, and so on, and said ranges are intended to include all whole numbers in between), to at least 6 contiguous nucleotides of any of the nucleic acid sequences comprised in the transcript, that can encompass the protein coding sequence region as well the non-coding or untranslated regions.

The miRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Publication 2004/0019001 and U.S. Pat. No. 6,673,611 (each of which is incorporated by reference in its entirety). Collectively, all such altered nucleic acids or RNAs described above are referred to as modified miRNAs.

In a most preferred embodiment, the miRNA hairpins comprised in the multiplexed miRNA expression construct of the present invention are constructed according to WO2019186274, in respect of the miRNA architecture and design described therein, which is incorporated by reference herein in its entirety. The skilled person understands how to apply the principles of miRNA design in the art and this reference to achieve optimal results with the multiplexed miRNA expression constructs of the present invention.

### Vectors for Cloning, Gene Transfer and Expression

Within certain aspects expression vectors are employed to express a nucleic acid of interest, such as a nucleic acid that inhibits the expression of a particular gene. Expression requires that appropriate signals be provided in the vectors, and which include various regulatory elements, such as enhancers/promoters from both viral and mammalian sources that drive expression of the genes of interest in host cells. Elements designed to optimize RNA stability in host cells also are defined. The conditions for the use of a number of dominant drug selection markers for establishing permanent, stable cell clones expressing the products are also provided, as is an element that links expression of the drug selection markers to expression of the polypeptide.

### Regulatory Elements

Throughout this application, the term “expression construct” or “expression vector” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a protein product. In other embodiments, expression only includes transcription of the nucleic acid encoding a gene of interest i.e., as is the case with RNA molecules of the embodiments.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for eukaryotic RNA polymerase (Pol) I, II or III. Much of the thinking about how promoters are organized derives from analyses of several viral Pol II promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins. At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In some embodiments, the promoter comprises an Elongation Factor 1 short (EF1s) promoter. In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof. In some aspects, a promoter for use according to the instant embodiments is a non-tissue specific promoter, such as a constitutive promoter.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that can be used in combination with the nucleic acid encoding a gene or miRNA of interest in an expression construct (Table 2 and Table 3). Additionally, any promoter/enhancer combination (as per the Eukaryotic Promoter Data Base EPDB) could also be used to drive expression of the gene or miRNA of interest. Truncated promoters may also be used to drive expression. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Elongation Factor 1 alpha (EF1α) | Kim et al., 1990 |
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al., 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., |
| | 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et |
| | | al., 1987, Karin et al., |
| | | 1987; Angel et al., 1987b; |
| | | McNeall et al., 1989 |
| MMTV (mouse mammary | Glucocorticoids | Huang et al., 1981; Lee et |
| tumor virus) | | al., 1981; Majors et al., |
| | | 1983; Chandler et al., |
| | | 1983; Ponta et al., 1985; |
| | | Sakai et al., 1988 |
| β-Interferon | poly(rl)x | Tavernier et al., 1983 |
| | poly(rc) | |
| Adenovirus 5 E2 | EIA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | EIA, SV40 Large T Antigen | Taylor et al., 1989, 1990a, |
| | | 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

Where any cDNA insert is employed, one will typically include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. In some aspects, however, a polyadenylation signal sequence is not included in a vector of the embodiments. For example, incorporation of such a signal sequence in lentiviral vectors (before a 3′ LTR) can reduce resulting lentiviral titers.

A spacer sequence may be included in the nucleic acid construct. The presence of a spacer appears to enhance knockdown efficiency of miRNA (Stegmeier et al., 2005). Spacers may be any nucleotide sequence. In some aspects, the spacer is GFP.

Also contemplated as an element of the expression cassette is a terminator. These elements can serve to enhance message levels and to minimize read through from the cassette into other sequences.

### Selectable Markers

In certain embodiments of the invention, the cells contain nucleic acid constructs of the present invention, a cell may be identified in vitro, ex vivo or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art. In an embodiment, the selectable marker is RQR8, tEGFR or TCD20.

### Delivery of Nucleic Acid Molecules and Expression Vectors

In certain aspects, vectors for delivery of nucleic acids of the embodiments could be constructed to express these factors in cells. In a particular aspect, the following systems and methods may be used in delivery of nucleic acids to desired cell types.

### Homologous Recombination

In certain aspects of the embodiments, the vectors encoding nucleic acid molecules of the embodiments may be introduced into cells in a specific manner, for example, via homologous recombination. Current approaches to express genes in stem cells have involved the use of viral vectors (e.g., lentiviral and gamma-retroviral vectors) or transgenes that integrate randomly in the genome. Some of these approaches, particularly the use of gamma-retroviral vectors, have been compromised in part due in part due to the random integration of the vectors that can activate or suppress endogenous gene expression, and/or the silencing of transgene expression. The problems associated with random integration could be partially overcome by homologous recombination to a specific locus in the target genome.

Homologous recombination (HR), also known as general recombination, is a type of genetic recombination used in all forms of life in which nucleotide sequences are exchanged between two similar or identical strands of DNA. The technique has been the standard method for genome engineering in mammalian cells since the mid-1980s. The process involves several steps of physical breaking and the eventual rejoining of DNA. This process is most widely used in nature to repair potentially lethal double-strand breaks in DNA. In addition, homologous recombination produces new combinations of DNA sequences during meiosis, the process by which eukaryotes make germ cells like sperm and ova. These new combinations of DNA represent genetic variation in offspring which allow populations to evolutionarily adapt to changing environmental conditions over time. Homologous recombination is also used in horizontal gene transfer to exchange genetic material between different strains and species of bacteria and viruses. Homologous recombination is also used as a technique in molecular biology for introducing genetic changes into target organisms.

Homologous recombination can be used as targeted genome modification. The efficiency of standard HR in mammalian cells is only 10-6 to 10-9 of cells treated (Capecchi, 1990). The use of meganucleases, or homing endonucleases, such as I-SceI have been used to increase the efficiency of HR. Both natural meganucleases as well as engineered meganucleases with modified targeting specificities have been utilized to increase HR efficiency (Pingoud and Silva, 2007; Chevalier et al., 2002). Another path toward increasing the efficiency of HR has been to engineer chimeric endonucleases with programmable DNA specificity domains (Silva et al., 2011). Zinc-finger nucleases (ZFN) are one example of such a chimeric molecule in which Zinc-finger DNA binding domains are fused with the catalytic domain of a Type IIS endonuclease such as FokI (as reviewed in Durai et al., 2005; PCT/US2004/030606). Another class of such specificity molecules includes Transcription Activator Like Effector (TALE) DNA binding domains fused to the catalytic domain of a Type IIS restriction endonuclease such as FokI (Miller et al., 2011: PCT/IB2010/000154).

### Nucleic Acid Delivery Systems

One of skill in the art would be well equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV etc), lentiviral vectors (e.g., derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

### Episomal Vectors

The use of plasmid- or liposome-based extra-chromosomal (i.e., episomal) vectors may be also provided in certain aspects of the invention, for example, for reprogramming of somatic cells. Such episomal vectors may include, e.g., oriP-based vectors, and/or vectors encoding a derivative of EBV-protein EBNA-1. These vectors may permit large fragments of DNA to be introduced to a cell and maintained extra-chromosomally, replicated once per cell cycle, partitioned to daughter cells efficiently, and elicit substantially no immune response.

In particular, EBNA-1, the only viral protein required for the replication of the oriP-based expression vector, does not elicit a cellular immune response because it has developed an efficient mechanism to bypass the processing required for presentation of its antigens on MHC class I molecules (Levitskaya et al., 1997). Further, EBNA-1 can act in trans to enhance expression of the cloned gene, inducing expression of a cloned gene up to 100-fold in some cell lines (Langle-Rouault et al., 1998; Evans et al., 1997). Finally, the manufacture of such oriP-based expression vectors is inexpensive.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi's sarcoma herpes virus (KSHV); Herpes virus saimiri (HS) and Marek's disease virus (MDV). Also other sources of episome-based vectors are contemplated, such as yeast ARS, adenovirus, SV40, or BPV.

One of skill in the art would be well equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

### Transposon-Based System

According to a particular embodiment the introduction of nucleic acids may use a transposon-transposase system. The used transposon-transposase system could be the well-known Sleeping Beauty, the Frog Prince transposon-transposase system (for the description of the latter see e.g., EP1507865), or the TTAA-specific transposon piggyback system.

Transposons are sequences of DNA that can move around to different positions within the genome of a single cell, a process called transposition. In the process, they can cause mutations and change the amount of DNA in the genome. Transposons were also once called jumping genes, and are examples of mobile genetic elements.

There are a variety of mobile genetic elements, and they can be grouped based on their mechanism of transposition. Class I mobile genetic elements, or retrotransposons, copy themselves by first being transcribed to RNA, then reverse transcribed back to DNA by reverse transcriptase, and then being inserted at another position in the genome. Class II mobile genetic elements move directly from one position to another using a transposase to “cut and paste” them within the genome.

### Viral Vectors

In generating recombinant viral vectors, non-essential genes are typically replaced with a gene or coding sequence for a heterologous (or non-native) protein or nucleic acid. Viral vectors are a kind of expression construct that utilizes viral sequences to introduce nucleic acid and possibly proteins into a cell. The ability of certain viruses to infect cells or enter cells via pH-dependent or pH-independent mechanisms, to integrate their genetic cargo into a host cell genome and to express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Retroviruses have promise as gene delivery vectors due to their ability to integrate their genes into the host genome, transferring a large amount of foreign genetic material, infecting a broad spectrum of species and cell types and of being packaged in special cell lines (Miller, 1992). In order to construct a retroviral vector, a nucleic acid is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid (i.e., the vector genome) to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Depending on the tropism of the envelope protein used to cover the vector particles surface, retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; Giry-Laterriere et al., 2011; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference.

### Nucleic Acid Delivery

Introduction of a nucleic acid, such as DNA or RNA, into cells to be programmed with the current invention may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by desiccation/inhibition mediated DNA uptake (Potrykus et al., 1985), and any combination of such methods. Through the application of techniques such as these, organelle(s), cell(s), tissue(s) or organism(s) may be stably or transiently transformed.

### Liposome Mediated Transfection

In a certain embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers (Ghosh and Bachhawat, 1991). Also contemplated is a nucleic acid complexed with Lipofectamine (Gibco BRL) or Superfect (Qiagen). The amount of liposomes used may vary upon the nature of the liposome as well as the cell used, for example, about 5 to about 20 μg vector DNA per 1 to 10 million of cells may be contemplated. Liposome mediated nucleic acid delivery and expression of foreign DNA in vitro has been very successful (Nicolau and Sene, 1982; Fraley et al., 1979; Nicolau et al., 1987). The feasibility of liposome mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

### Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L (A9), mouse C127, CHO, CV-1, BHK, NIH3T3 and Hela cells were transfected with a neomycin marker gene (Chen and Okayama, 1987), and rat hepatocytes were transfected with a variety of marker genes (Rippe et al., 1990).

### DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

### Cell Culturing

Generally, cells of the present invention are cultured in a culture medium, which is a nutrient-rich buffered solution capable of sustaining cell growth.

Culture media suitable for isolating, expanding and differentiating stem cells according to the method described herein include but are not limited to high glucose Dulbecco's Modified Eagle's Medium (DMEM), DMEM/F-12, Liebovitz L-15, RPMI 1640, Iscove's modified Dubelcco's media (IMDM), and Opti-MEM SFM (Invitrogen Inc.). Chemically Defined Medium comprises a minimum essential medium such as Iscove's Modified Dulbecco's Medium (IMDM) (Gibco), supplemented with human serum albumin, human Ex Cyte lipoprotein, transferrin, insulin, vitamins, essential and non-essential amino acids, sodium pyruvate, glutamine and a mitogen is also suitable. As used herein, a mitogen refers to an agent that stimulates cell division of a cell. An agent can be a chemical, usually some form of a protein that encourages a cell to commence cell division, triggering mitosis. In one embodiment, serum free media such as those described in U.S. Ser. No. 08/464,599 and WO96/39487, and the “complete media” as described in U.S. Pat. No. 5,486,359 are contemplated for use with the method described herein. In some embodiments, the culture medium is supplemented with 10% Fetal Bovine Serum (FBS), human autologous serum, human AB serum or platelet rich plasma supplemented with heparin (2 U/ml). Cell cultures may be maintained in a CO₂ atmosphere, e.g., 5% to 12%, to maintain pH of the culture fluid, incubated at 37° C. in a humid atmosphere and passaged to maintain a confluence below 85%.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

Other objects, features and advantages of the present invention are apparent from the Examples. It should be understood, however, that the detailed description and the specific examples, while indicating certain embodiments of the invention, do not limit the present invention and are given merely by way of illustration, since the present invention is defined by the accompanying claims.

### EXAMPLES

### Example 1: Methodology

Briefly, the approach of the present inventors to developing optimal gene silencing constructs includes, (i) target sequence design, (ii) molecular cloning to create single hairpin miRNA constructs and lentiviral vector production, and (iii) gene modification of target cells and assessment of gene/protein silencing using appropriate read-out methods. Optimal target sequence(s) may then be used to create multi-hairpin miRNA constructs to similarly assess for maximal gene silencing efficiencies.

### Target Sequence Design

Target sequences for incorporation into miRNA architecture were designed using software which rationally prioritizes the selection of optimal gene silencing target sequences based on known parameters. Sequence prioritization is based on identifying conserved regions in target transcripts, individually scoring each target sequence and mitigating the risk of potential off-target gene silencing (based on sequence identity between the target sequence and the transcriptome of the target cells). The notation “TARGET NAME_T#” (e.g. TRAC_T1, B2M_T5) is used to refer to target sequences in genes, and the associated miRNA targeting those sequences.

### Molecular Cloning

Prioritized target sequences were synthesized within a mirGE backbone by a third-party manufacturer. The single mirGE sequences were cloned using LR Clonase II. The mirGE pENTR plasmid, an elongation factor 1 short promoter (pENTR-L4-EFs-L1R) plasmid and a lentivector destination cassette (pCWX-R4dESTR2-PC) containing the mCherry reporter gene were cloned into a single plasmid. Successful cloning of all constructs was confirmed via restriction enzyme digestion pattern and DNA sequencing.

### Lentiviral Vectors and Titration

Lentiviral vectors were produced by transfecting HEK293T cells with transfer plasmids carrying the gene silencing construct, as well as lentiviral packaging (PAX2) and envelope (VSVg) plasmids. The cell culture medium was replenished after 4-6 hours and subsequently harvested at 24 hours for viral particle collection. The culture medium was collected, filtered to remove cellular debris, and viral particles enriched using PEG-It Virus Precipitation Solution (System Biosciences), according to the manufacturer's instructions. Final aliquots of concentrated lentiviral vectors were stored at −80° C. Functional viral vector titers were assessed by transducing primary T-cells over a range of dilutions and measuring the percentage of cells expressing mCherry reporter gene.

### Cells and Cell Lines

Silencing of HLA-I, HLA-II and TCR cell surface expression was assessed in Jurkat cells and primary T-cells, which were prepared from anonymized buffy coat blood units procured from the Blood Transfusion Centre of the University Hospital of Geneva, Switzerland. The peripheral blood mononuclear cells (PBMCs) were isolated using Ficoll separation, after which T-cells were separated using Miltenyi CD4/CD8 microbeads and cryopreserved in aliquots in liquid nitrogen. HEK293T cells were also used to test some miRNA constructs.

### Lentiviral Vector Transduction

Cryopreserved T-cells were thawed, cultured overnight in T-cell medium (Advanced RPMI, 10% FBS), and activated the following day using CD3/CD28 Dynabeads at a ratio of 1:1. Activated T-cells were transduced 48 hours later with lentiviral vectors carrying the miRNA gene silencing constructs. Transductions were performed in high density volumes (2 million cells per mL), and the medium replenished after 18-24 hours and every other day thereafter for T-cell maintenance at a cell density 1 million/mL. For testing in HEK293T cells, multiplicity of infections (MOIs) of 0.2 and 2.0 were used derived cells with a range of HLA class I silencing. Following transduction, the HEK293T cells were maintained in appropriate media for a minimum of five days prior to assessing silencing of HLA-ABC cell surface expression. For assessment in Jurkat cells, target cells were transduced and maintained in appropriate media for a minimum of five days prior to assessing silencing of target gene expression.

### Flow Cytometry and Assessment of Gene Silencing

Flow cytometry was performed at 5-7 days post-transduction of T-cells. Cells were harvested, washed, resuspended in PBS solution, and stained for 20-30 min with the appropriate antibodies for assessment of cell surface expression. Following staining, cells were washed with PBS, resuspended in FACS buffer (Ca/Mg2+ Free PBS, 2 mM EDTA, 0.5% BSA), and cell surface expression assessed via flow cytometry. Captured data were exported to FlowJo for analysis. To calculate the level of gene silencing, changes in both the percentage of cells positive for the target and median florescence intensity (MFI) were assessed. Normalization includes expression levels within samples (modified vs unmodified cells), after which expression levels relative to the control-transduced cells were calculated.

### Mixed Lymphocyte Reactions

miRNA constructs were used to transduce primary T-cells and tested for functional silencing in mixed lymphocyte reactions (MLRs) with unmatched PBMCs (stimulator cells). Unmodified T-cells were expected to be alloreactive against stimulator cells, while TCR-silenced T-cells were expected to not be activated when co-cultured with unmatched T-cells. Stimulator cells were irradiated and labelled with PKH26 prior to co-culture. Responder cells (TCR-silenced cells) were generated and brought to resting state over 12 days of cell culture and the removal of IL-2. The cells were then co-cultured at a ratio of 1:1 and T-cell activation assessed via flow cytometry for the expression of CD137 in CD8+ T-cells. A positive control group activated with CD3/CD28 microbeads (1:1) was also included. Responder cells were then brought to resting state again and similarly re-stimulated.

### Example 2: HLA-I Down-Regulation and B2M Expression Tuning with miRNA

The major histocompatibility complex (MHC), or human leukocyte antigen (HLA) class I receptors—namely, HLA-A, HLA-B and HLA-C—are expressed on the surface of all nucleated cells in the body. Together with HLA class II, they play a central role in the presentation of peptide antigens to the immune system, which is recognized by the T-cell receptor (TCR) on T-cells. Peptides presented by HLA class I and class II receptors are recognized by CD8+ and CD4+ T-cells, respectively. Beta-2 microglobulin (B2M) is a common protein subunit of all HLA class I molecules.

In the context of allogeneic chimeric antigen receptor (CAR) T-cell therapies, besides the need to silence or knockout the TCR to prevent alloreactivity of CAR T-cells, it is also important to prevent these donor-derived CAR T-cells from being rejected by the recipient's immune system. HLA class I expression is up-regulated in activated CAR T-cells and may lead to rejection directed via CD8+ T-cells. However, cells which do not express HLA class I may lead to rejection directed via NK cells.

The present inventors proposed a strategy of using miRNA to down-regulate B2M, and thus all HLA class I molecules, using miRNA. This strategy was proposed to improve persistence of allogeneic CAR T-cells by avoiding CD8+ T-cell mediated cytotoxicity. However, the present inventors further surprisingly found that their miRNA-based approach could be used to “tune” B2M expression, thereby maintain a beneficial low level of HLA class I. Thus, this miRNA-based approach also improves persistence of allogeneic CAR T-cells by avoiding the possibility of NK cell mediated cytotoxicity. In addition, the miRNA construct used to silence HLA class I could also be used for creating universal donor cells from various sources, including induced pluripotent stem cells (iPSCs).

With typical gene editing approaches, HLA-I is completely deleted and thus donor cells are prone to depletion by NK cells. To prevent this, non-classical HLA-I molecules (HLA-E or HLA-G) may be added to be co-expressed and to improve persistence. However, the miRNA-based approach of the present invention which achieves tunable (e.g. 70-90%) silencing of HLA in fact provides adequate or even superior persistence of allogeneic miCAR T-cells, without the need for co-expression of HLA-E/G.

### Gene Silencing in Target Cells

Target sequences were screened for based on the methodology previously described in Myburgh et al. (2014-PMID: 25350582). Target sequences were in respect of human B2M (ENSEMBL: ENSG00000166710). From this, miRNA designated as B2M_T5, B2M_T2 and B2M_T3 were obtained.

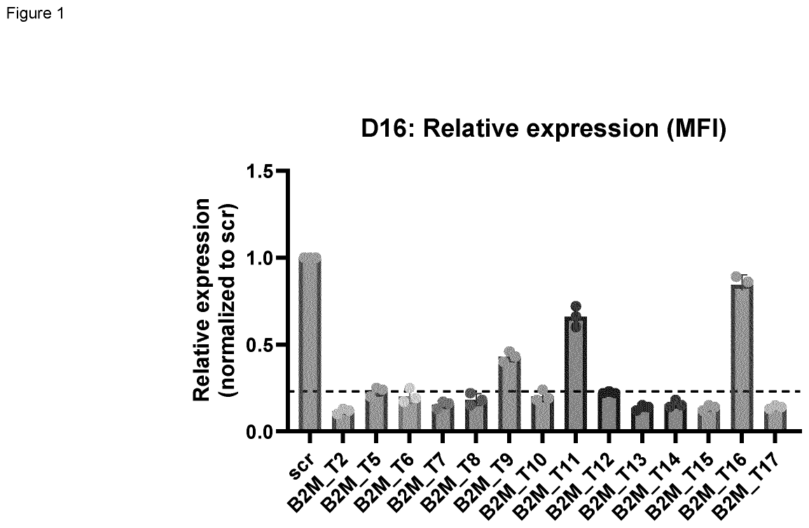

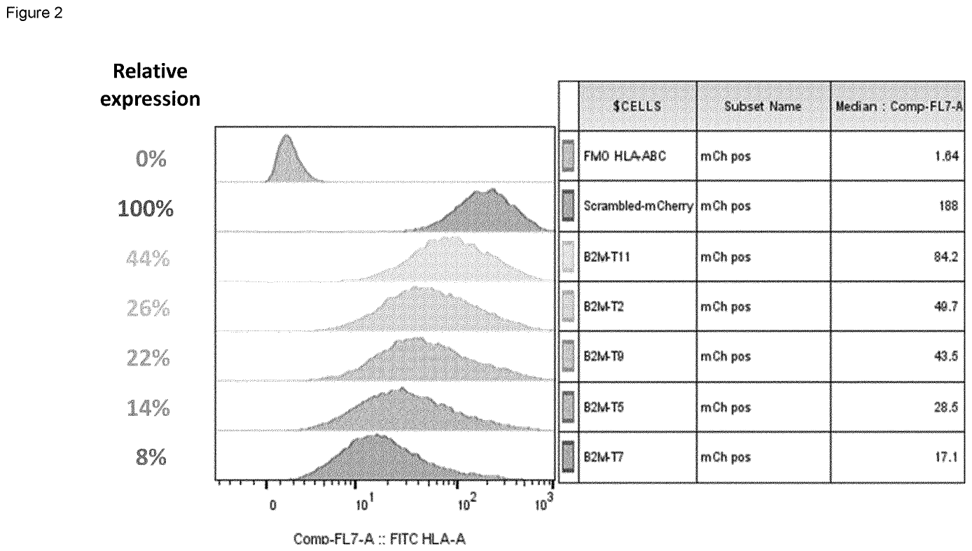

Sequences were tested based on downregulation of HLA-ABC expression in gene-modified primary T-cells. Cryopreserved T-cells were thawed, and activated using Thermo Fischer Scientific Dynabeads (at a 3:1 bead-to-cell ratio) and resuspended in T-cell media. Activated T-cells were transduced 24 hours later with lentiviral vectors carrying the miRNA gene silencing constructs. Transductions were performed in high density volumes (2 million cells per mL), and the medium replenished after 16-24 hours and every other day thereafter for T-cell maintenance at a cell density 1 million/ml. Flow cytometry was performed at 5 days post-transduction of T-cells. Results shown in FIG. 1 (down-regulation of HLA-ABC expression from a range of silencing constructs) and FIG. 2 (achieving varied HLA class I expression).

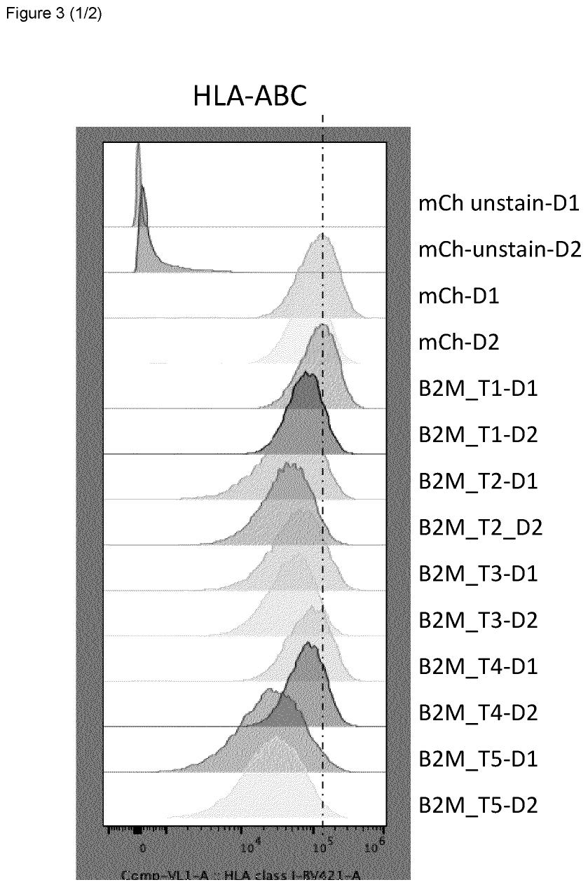

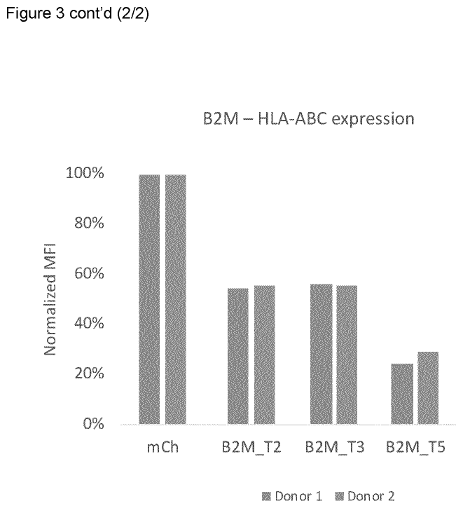

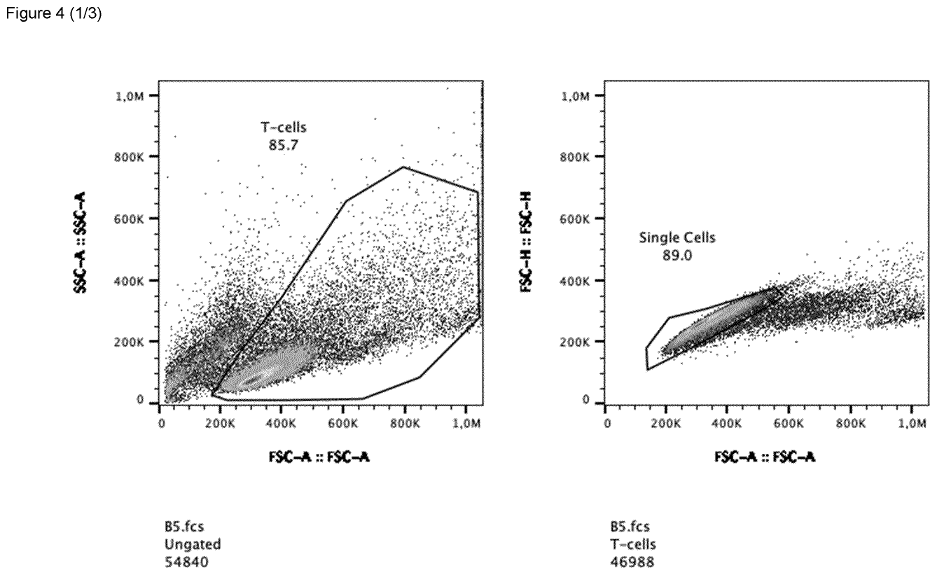

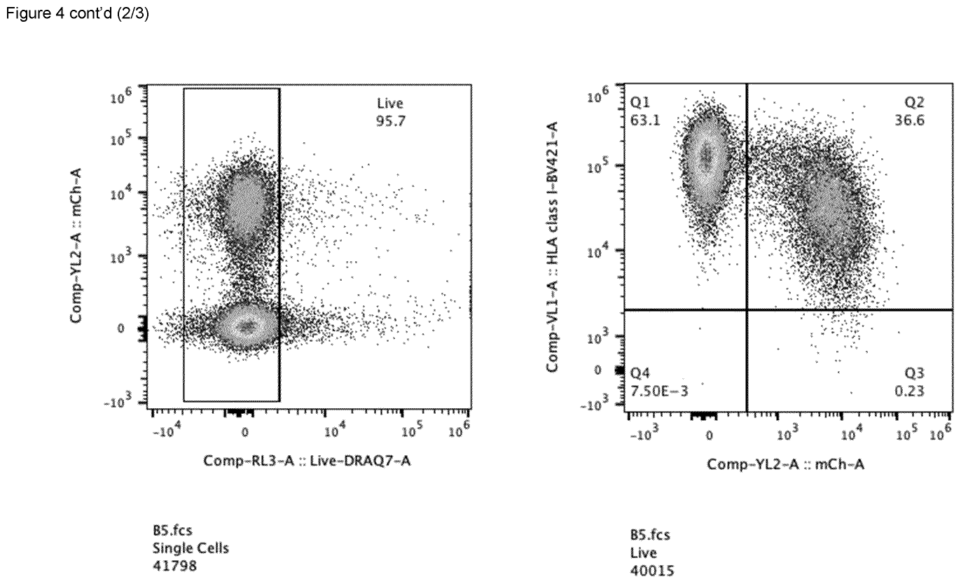

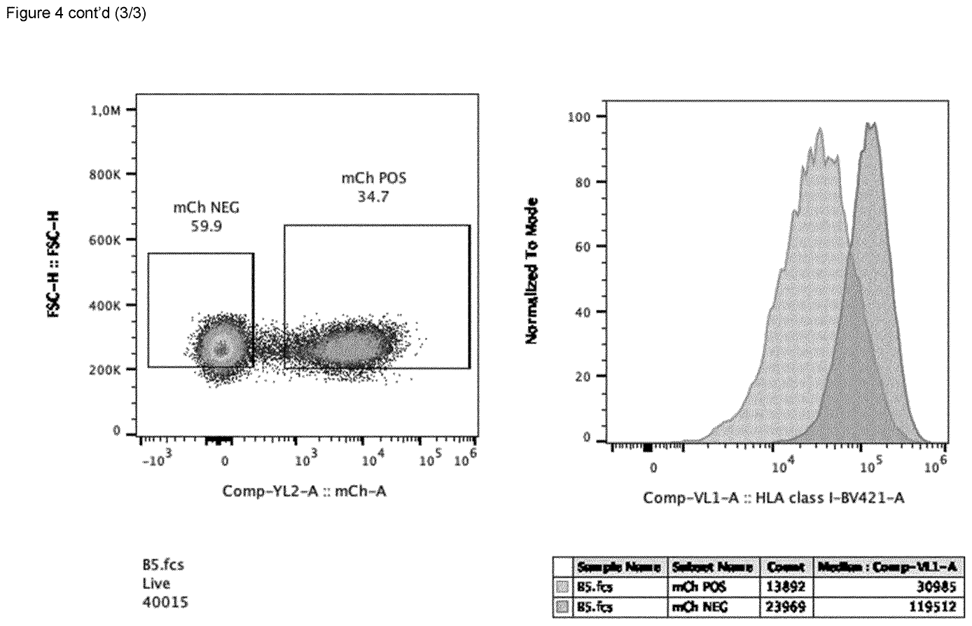

To further assess the efficiency of B2M gene silencing, primary T-cells were transduced with lentiviral vectors carrying each single hairpin miRNA construct, for which the data is shown in FIG. 3. In these experiments, T-cells from two donors were transduced. Gene silencing of HLA class I resulted in 50-75% reduced expression in gene-modified cells. All cells were transduced by 30-40%, and thus the data reflects HLA class I silencing from 1-2 vector copy numbers. Given the high efficiency HLA class I silencing achieved, future construct developments were pursued. The gating strategy for these assessments using B2M_T5 as an example is shown in FIG. 4.

### Multi-Hairpin Gene Silencing of Target in Gene-Modified Cells

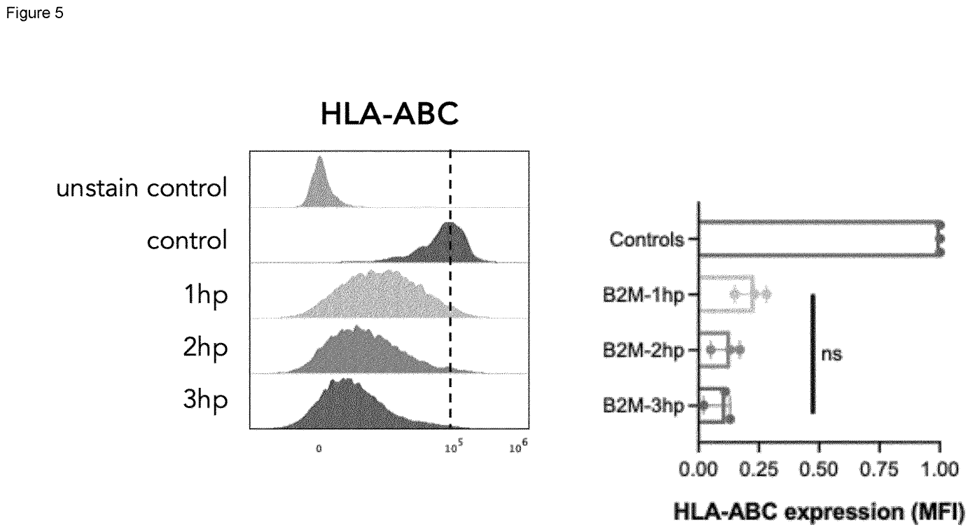

Having identified B2M_T5 as an efficient target sequence for silencing of HLA class I expression, it was next incorporated into multi-hairpin miRNA constructs (1 hp, 2 hp and 3 hp) to assess if incremental improvements in HLA class I silencing could be made. As indicated in FIG. 5, an improvement in target silencing was observed when using a 2 hp construct, while there is only a minor benefit beyond two hairpins.

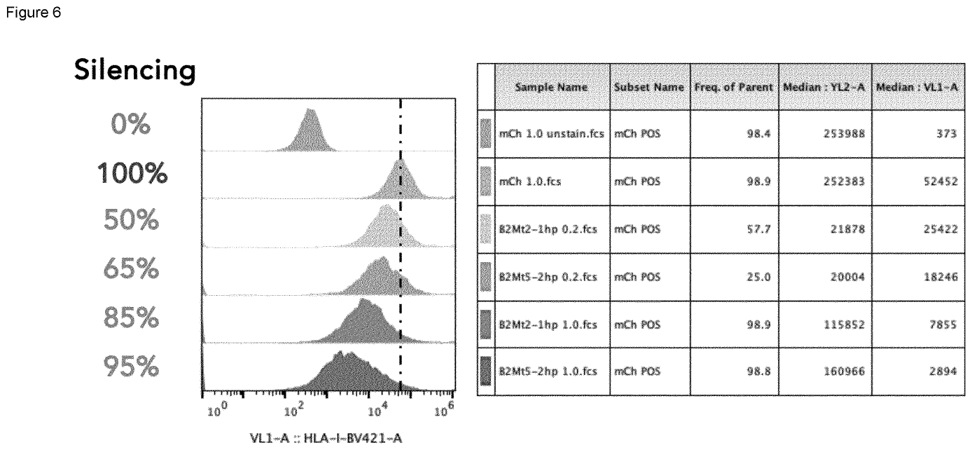

In preparation for future functional studies of CAR T-cell persistence, the ability to engineer HEK293 cells with varied HLA class I expression using different B2M gene silencing constructs was explored. These included single and dual hairpin miRNA constructs, targeting either the B2M_T2 or T5 target sequences, over 0.2 and 1.0 Mol lentiviral vector transductions. As illustrated in FIG. 6, it was possible to gene engineered HEK293 cells with HLA class I expression over a range of 50-95%. This demonstrates the ability to uniquely regulate expression levels of a target gene of choice using this miRNA gene silencing technology.

### Conclusions

Efficient gene silencing of HLA class I molecules via a miRNA construct targeting B2M was determined. With a single hairpin construct and low copy number transduction, it was possible to silence HLA class I by 50-75%. If the intention is to silence HLA class I expression by >90%, a 2 hp configuration, targeting multiple transcript sequences and/or higher transduction rates (60-70%) to increase miRNA copy numbers can be used. This technology offers a unique solution to “tune” HLA class I silencing to an intended amount between 50% and 90%.

### Example 3: TCR Down-Regulation and TRAC/CD3z Silencing with miRNA

The present inventors developed a highly effective miRNA gene construct capable of high efficiency gene silencing. Using this approach, the present inventors developed TCR-deficient T-cells, and demonstrated functional loss of alloreactivity in both in vitro and in vivo models.

TRAC is the constant region of T-cell receptor (TCR) alpha chain (TCRa) (PubMed: 24600447). Alpha-beta T-cell receptors are antigen specific receptors, which are essential to the immune response and are present on the cell surface of T-lymphocytes. Together with CD3, the TCR-CD3 complex is the definitive receptor of T-cells. The TCR-complex is formed through non-covalent association of eight subunits, namely one each of TCRa, TCRb, CD3g and CD3d, and two each of CD3e and CD3z. When antigen presenting cells (APCs) activate T-cell receptor (TCR), TCR-mediated signals are transmitted across the cell membrane via the CD3 complex. This is a prerequisite for efficient T-cell adaptive immunity against pathogens (PubMed: 25493333).

Cluster of differentiation 37 (CD3z), or CD247, is one of the components of the CD3 complex. All CD3 chains contain immunoreceptor tyrosine-based activation motifs (ITAMs) in their cytoplasmic domain. Upon TCR engagement, these motifs become phosphorylated by Src family protein tyrosine kinases LCK and FYN, resulting in the activation of downstream signaling pathways (PubMed: 2470098, PubMed: 7509083). CD3z ITAM phosphorylation creates multiple docking sites for the protein kinase ZAP70 leading to ZAP70 phosphorylation and its conversion into a catalytically active enzyme (PubMed: 7509083). CD3z also plays an important role in intra-thymic T-cell differentiation.

In the context of allogeneic CAR T-cell therapy, it is advantageous to limit alloreactivity of donor-derived CAR T-cells for the risk of GvHD. Alloreactivity is mediated via the TCR, and thus gene silencing of the TCR was investigated for the provision allogenic CAR T-cells. In this regard, the present inventors proposed to investigate the use of miRNA constructs against select subunits of the TCR, including TRAC and CD3z.

TRAC Silencing with miRNA

Target sequences in respect of human TRAC (ENSEMBL: ENSG00000277734) were screened for, prioritizing sequences starting with a thymidine (T) residue. From this, miRNA against TRAC target sequences designated as TRAC_T1, TRAC_T4 and TRAC_T5 were obtained. To assess the efficiency of TRAC gene silencing, PBMCs were transduced with lentiviral vectors carrying single hairpin miRNA constructs, followed by flow cytometric analysis of TCR a/b and CD3e expression.

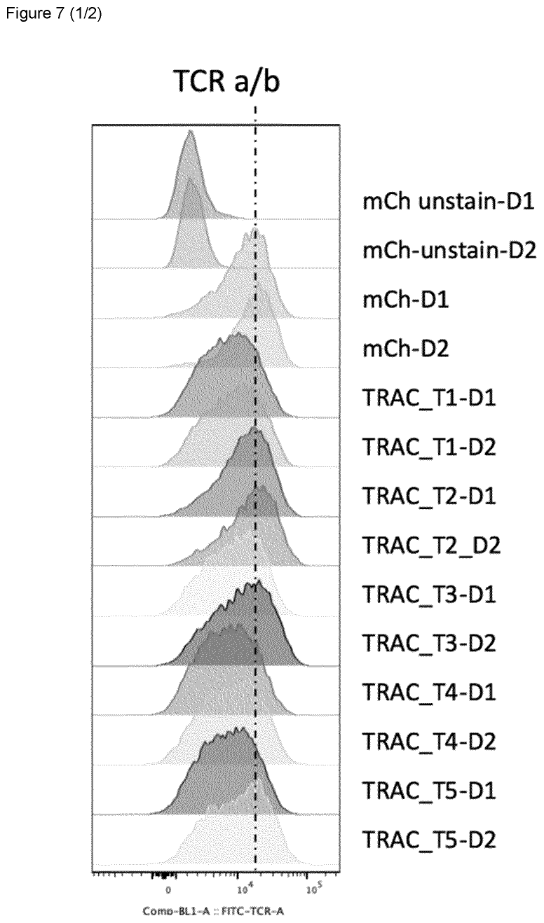

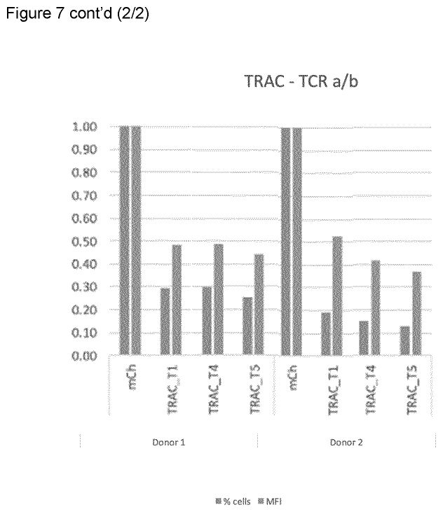

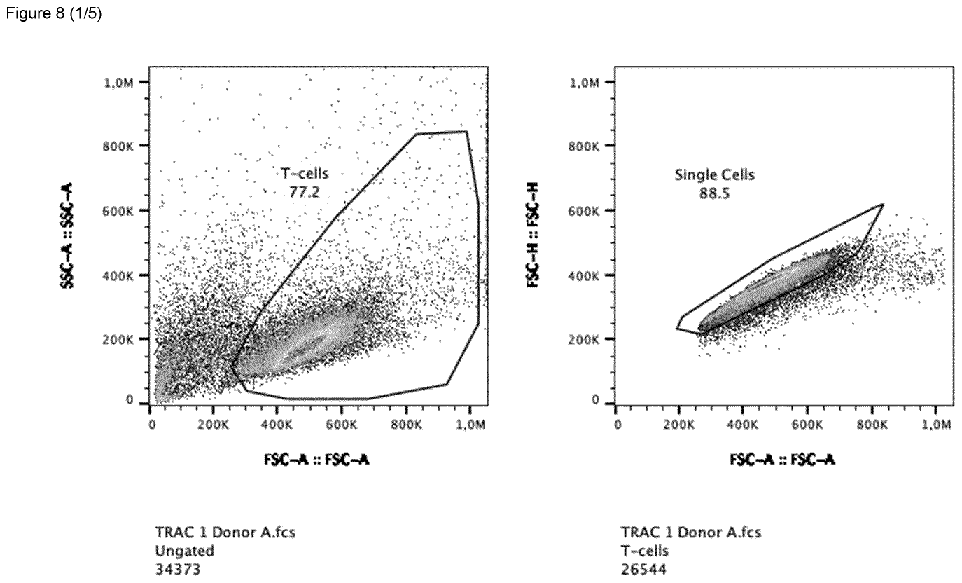

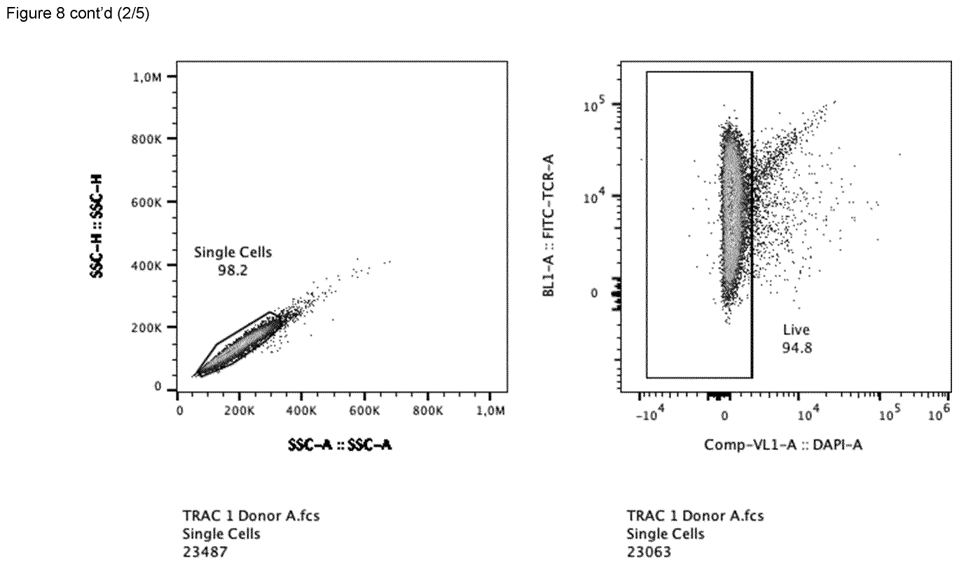

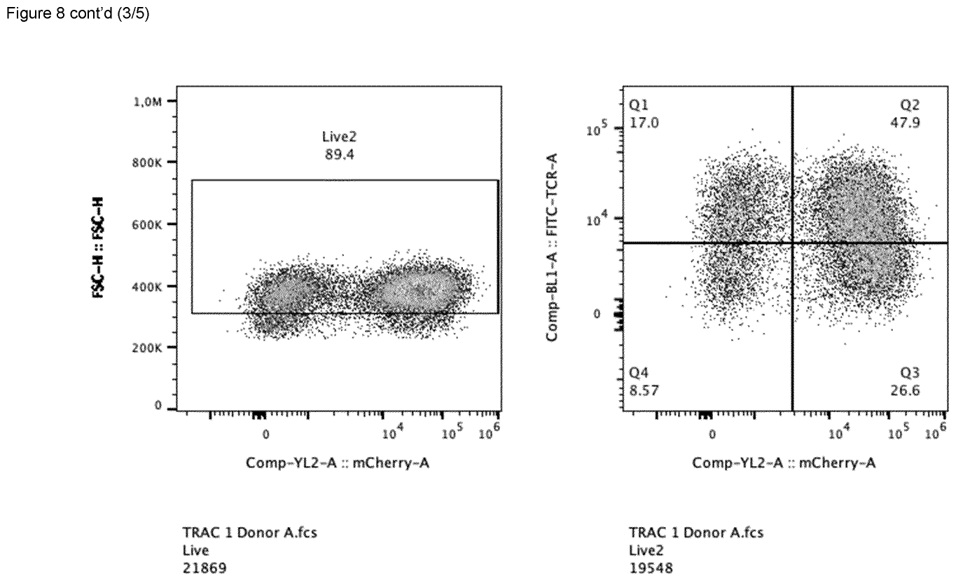

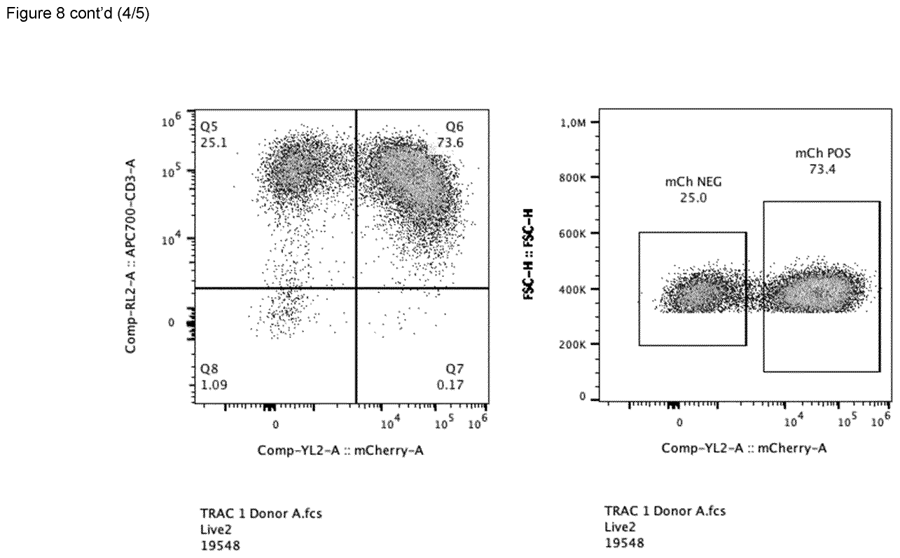

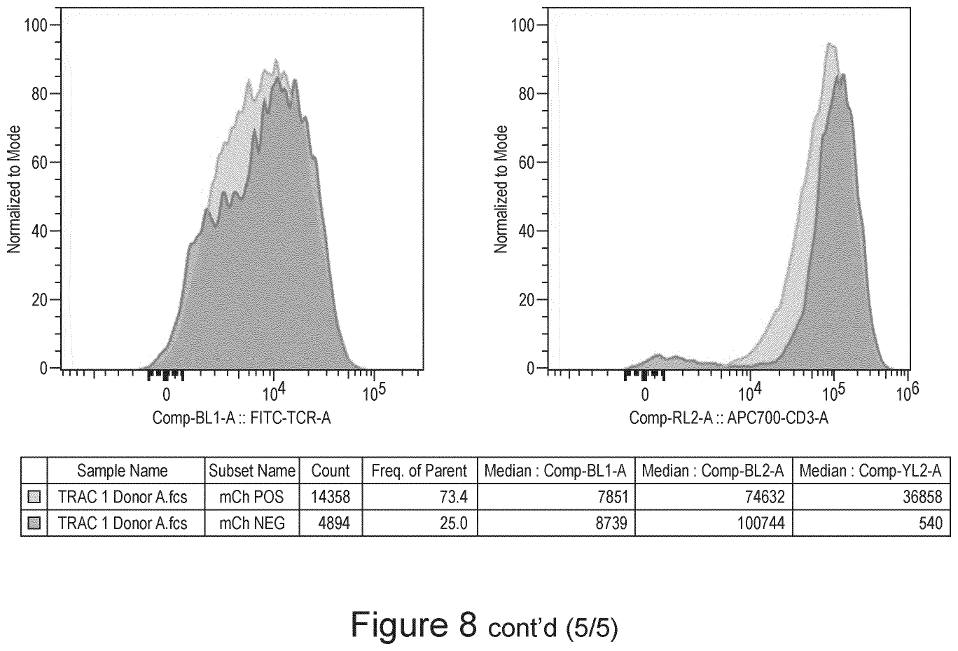

As illustrated in FIG. 7, TRAC_T1, TRAC_T4 and TRAC_T5 target sequences all resulted in a greater than 50% silencing of TCR expression (based on MFI readings normalized to the mCherry control). Moreover, there was a more than 70% decrease in the percentage of cells expressing TCR. Notably, the trend in gene silencing from the different TRAC targeting constructs remained consistent between both Donor 1 and 2 samples, with the efficiencies being slightly higher in Donor 2. All samples were transduced equally in a range of 55-65%, which reflects 2-3 vector copy numbers across the majority of gene-modified T-cells. The gating strategy for assessing TCR silencing is presented in FIG. 8.

In conclusion, gene silencing of TRAC was achieved using miRNA constructs. The three target sequences tested were shown to silence TCR expression by greater than 50%, which also translated to a 70% decrease in TCR-expressing cells. In the context of allogeneic T-cell therapies, and the potential risks associated graft vs host disease, it would be further advantageous to achieve >90% silencing of the TCR. This could be achieved by multiplexing miRNA hairpins against TRAC and/or against other subunits of the TCR-CD3 complex. miRNA constructs against CD3z have also been developed, for which TCR silencing data is reported below.

CD3z Silencing with miRNA

CAR T-cells commonly comprise chimeric antigen receptors that are designed to include a CD3z activation domain. The present inventors therefore identified CD3z target sequences outside of this domain to prevent the miRNA constructs from silencing CAR expression. Target sequences were based on the encoding human CD247 (ENSEMBL: ENSG00000198821). Three miRNA targeting CD3z, designated CD3z_T1, CD3z_T2 and CD3z_T3, were obtained in this manner.

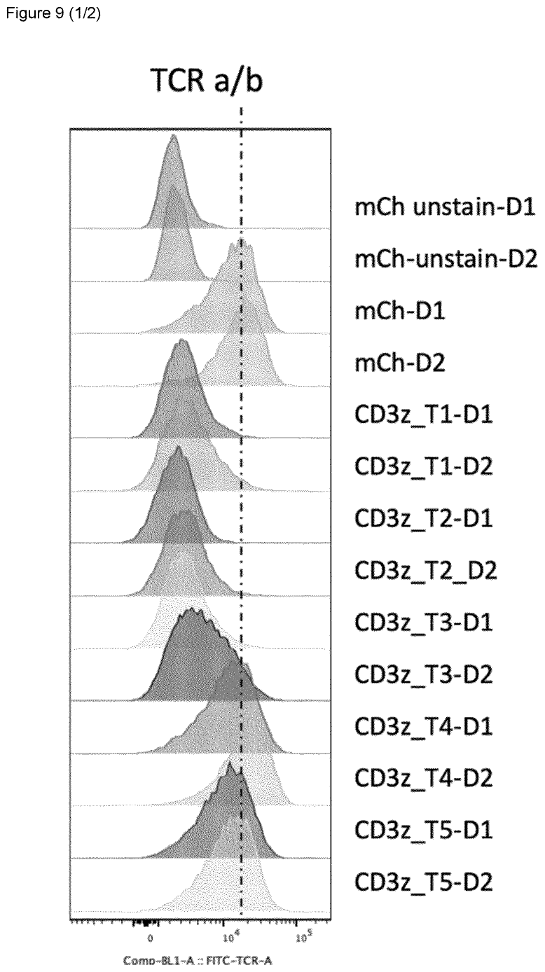

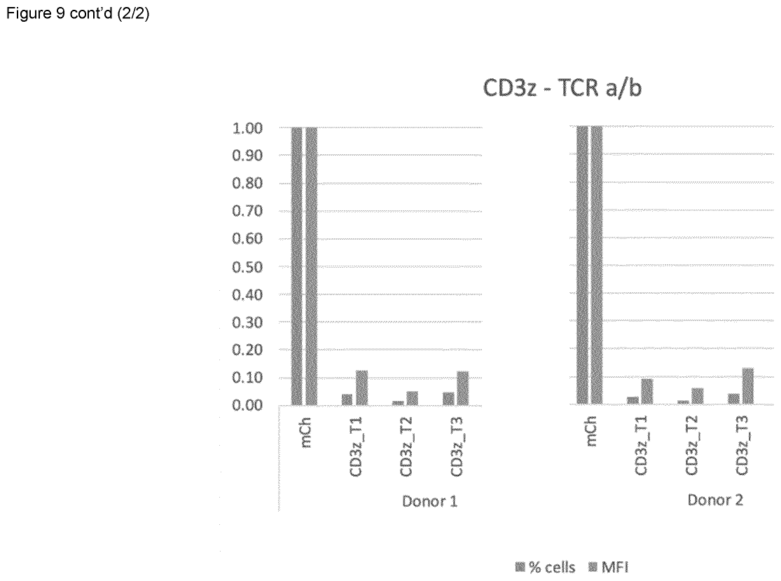

To assess the efficiency of CD3z gene silencing, PBMCs were transduced with lentiviral vectors carrying single hairpin miRNA constructs, followed by flow cytometric analysis of TCR a/b and CD3e expression. Data from an experiment using PBMCs from two healthy donors is presented in FIG. 9.

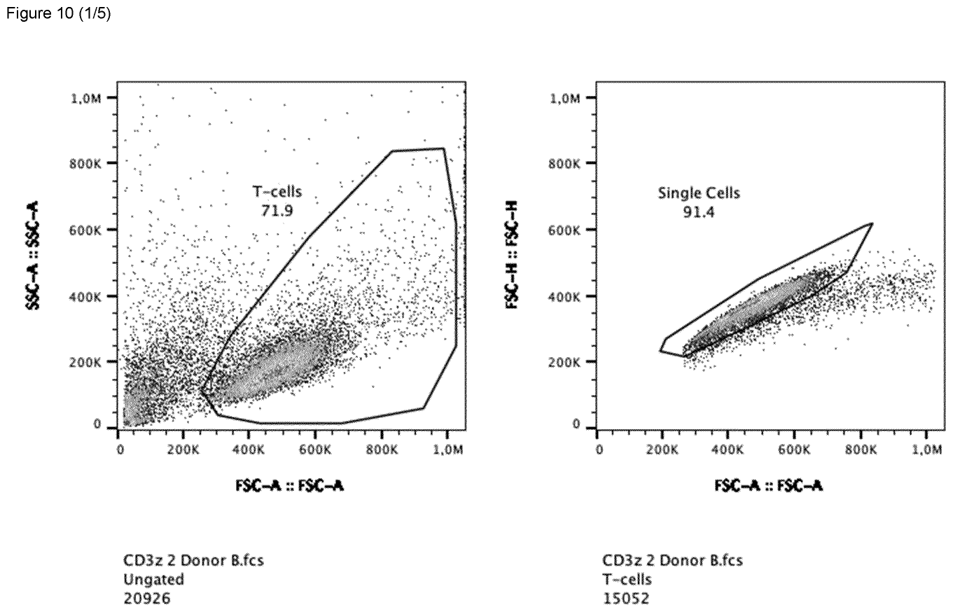

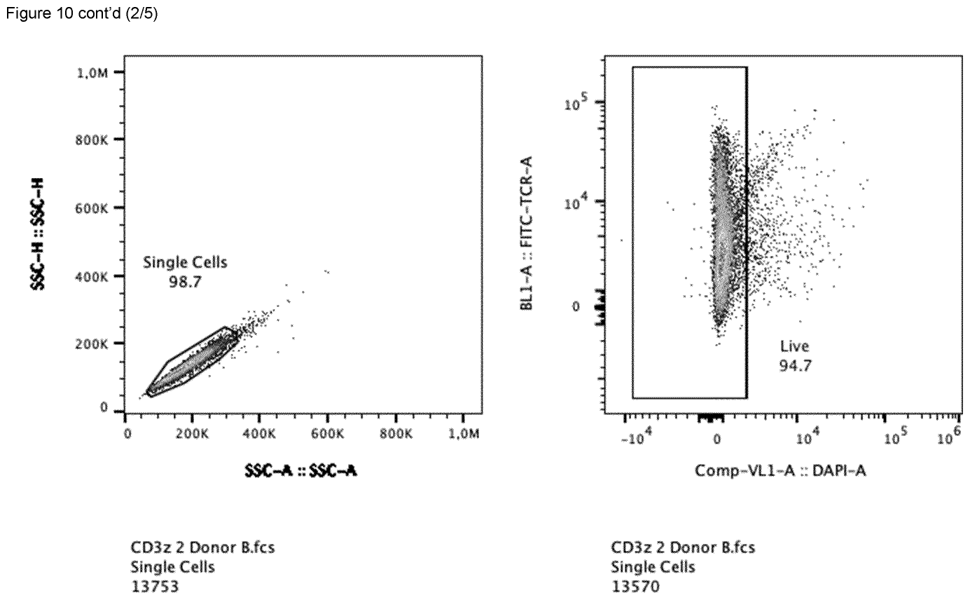

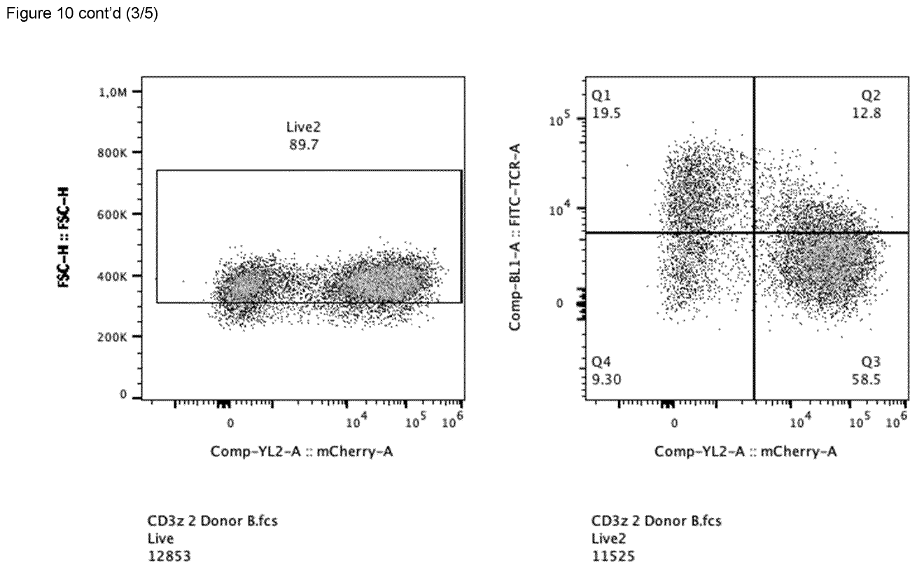

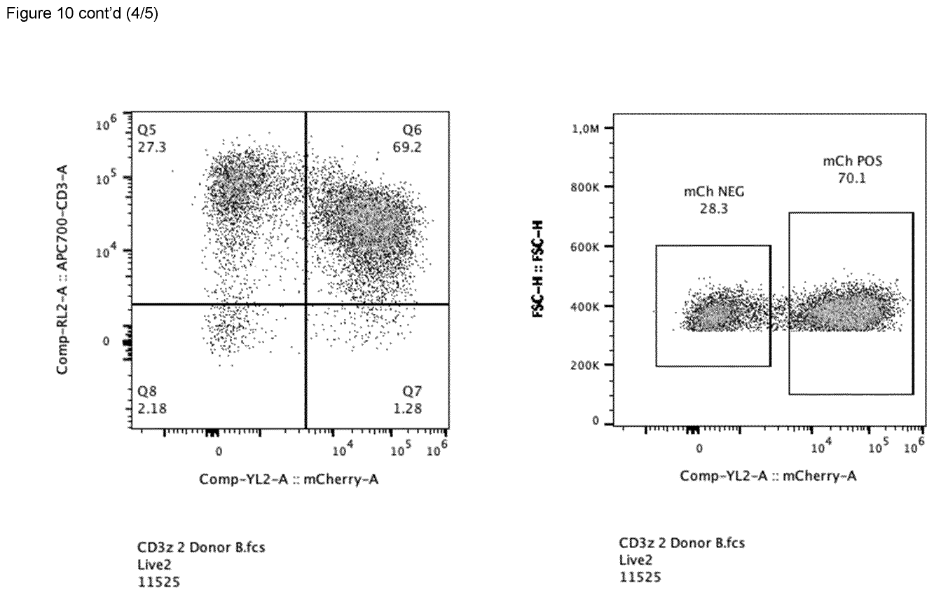

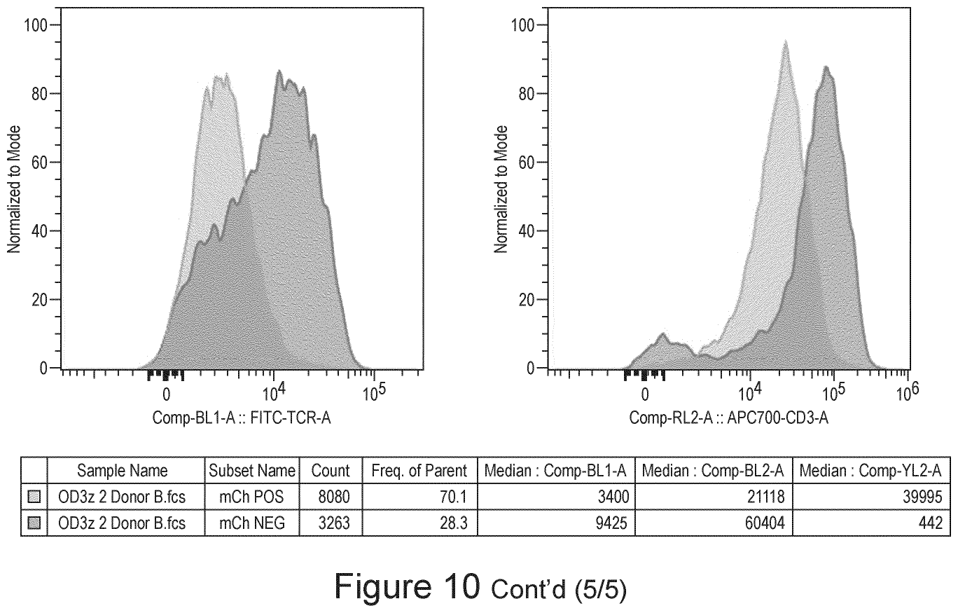

CD3z_T1, CD3z_T2 and CD3z_T3 all resulted in high efficiency silencing of TCR expression. The CD3z_T2 sequence appeared to be the most efficient with >95% silencing of TCR expression and a similar decrease in the percentage of TCR-expressing cells. The trend in gene silencing was consistent between both Donor 1 and 2 samples. The gating strategy for assessing TCR silencing is presented in FIG. 10.

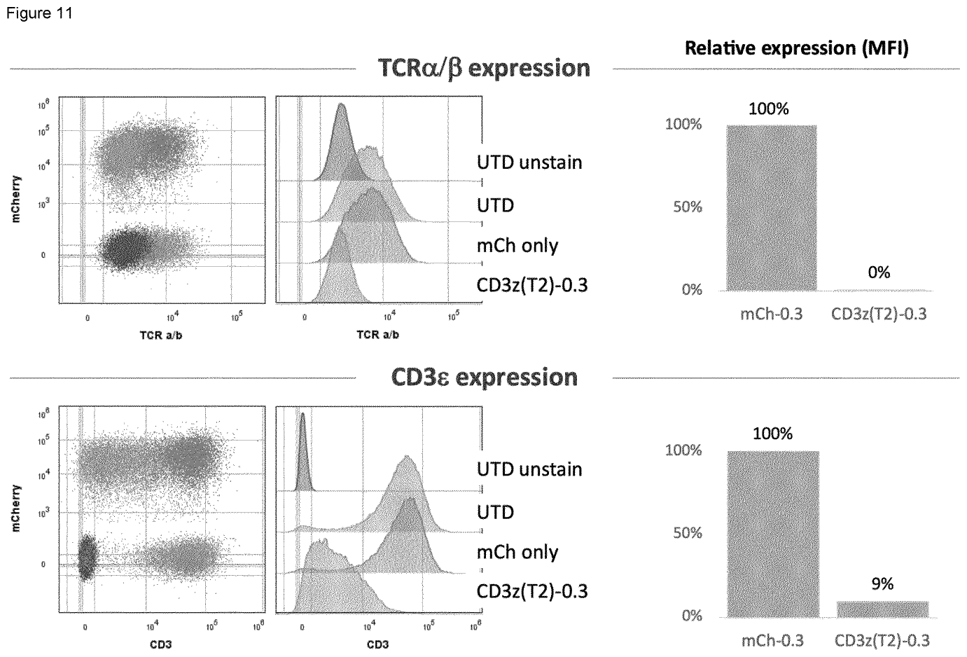

Given the high efficiency TCR silencing achieved with CD3z miRNA, future construct developments were pursued. As a next step, Jurkat cells were transduced to confirm the efficiency of TCR silencing. As shown in FIG. 11, Jurkat cells modified with a CD3z_T2 construct at a MOI of 0.3 completely ablated TCR a/b expression.

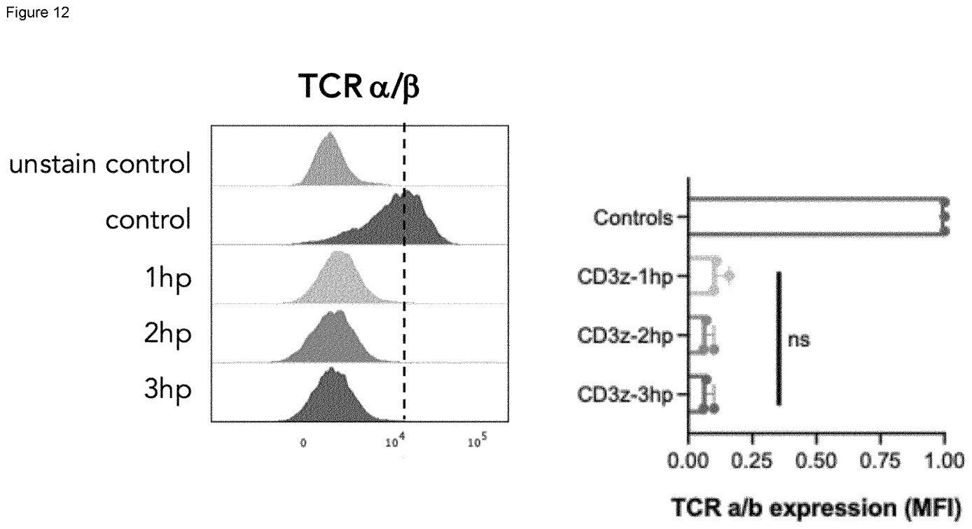

Having identified an efficient target for silencing of TCR expression, CD3z miRNA was next incorporated into multi-hairpin miRNA constructs (1 hp, 2 hp and 3 hp) to assess if incremental improvements in TCR silencing could be made. As indicated in FIG. 12, an increase in TCR silencing was observed when using a 2 hp construct against the same CD3z_T2 sequence.

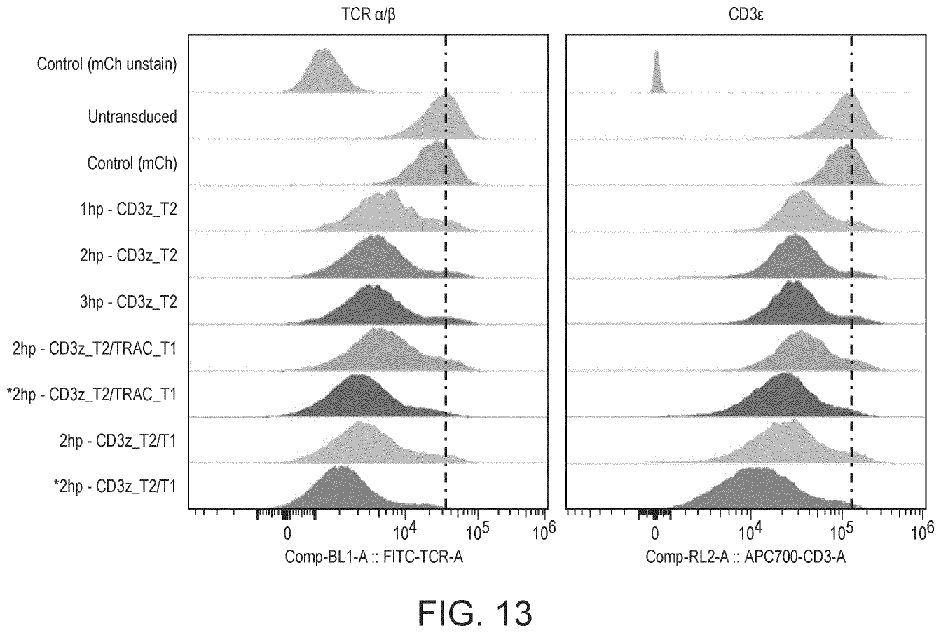

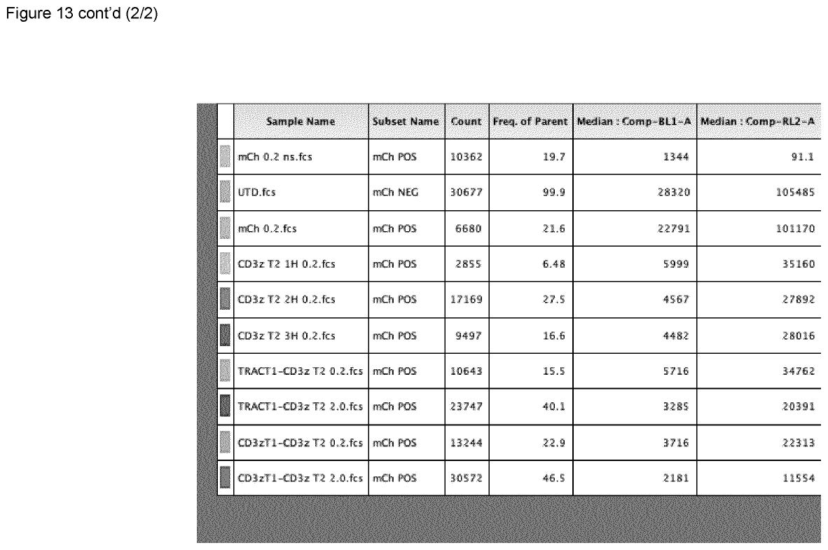

In addition to the multiple “hairpin dosage” testing of 1-3 hp of CD3z_T2 in the above experiments, two dual-hairpin constructs targeting different transcript regions were also created. In the first construct, a combination of CD3z_T2 and CD3z_T1 targeting miRNAs was used. A second construct targeting CD3z_T2 and TRAC_T1 was also created. To assess the efficiency of these novel constructs primary T-cells were transduced and both TCR a/b and CD3e expression were evaluated (FIG. 13). Also included were the 1-3 hp constructs against CD3z_T2. Based on the histograms and median fluorescence intensities (MFI) provided in FIG. 13, it is evident that the dual hairpin construct with two different CD3z sequence targets (CD3z_T2/T1) was the most efficient at silencing cell surface expression of the TCR-CD3 complex. Even when compared to a 3 hp CD3z_T2 construct, a visible decrease in both TCR a/b and CD3e expression was observed. This indicates that a miRNA construct comprising two miRNA hairpins targeting different sequences on CD3z is able to provide increased levels of gene silencing over a construct comprising two miRNA hairpins that only target a single (the same) CD3z sequence. Notably, at a higher transduction rate of 46.5% (denoted by the asterisk), >95% of transduced cells were completely negative for TCR expression. Also, the CD3z_T2/TRAC_T1 construct performed better than 3 hp CD3z_T2 when cells were transduced at 40.1%. These data demonstrate successful TCR silencing with a range of miRNA constructs, including complete silencing of TCR expression using CD3z_T2/T1. Any remaining cells expressing residual TCR will be removed following a TCR-based depletion.

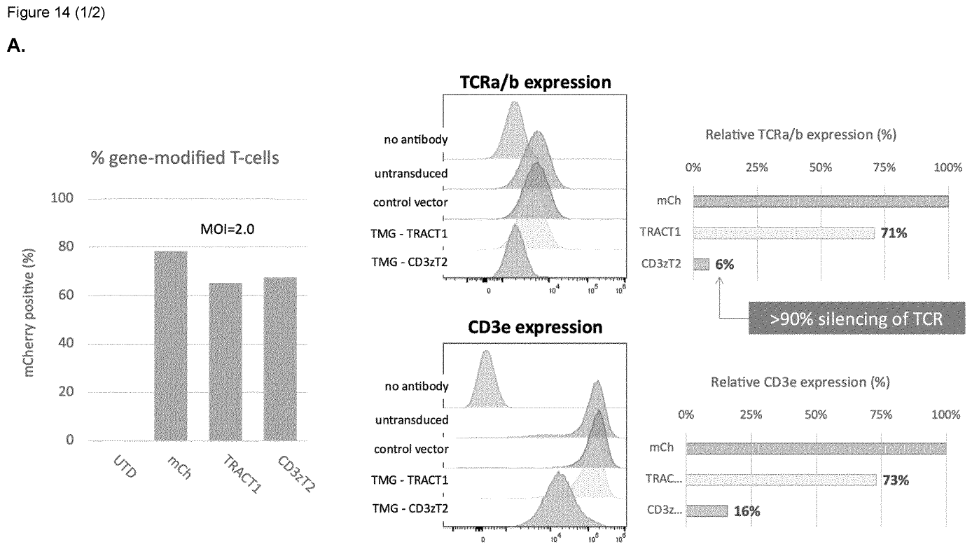

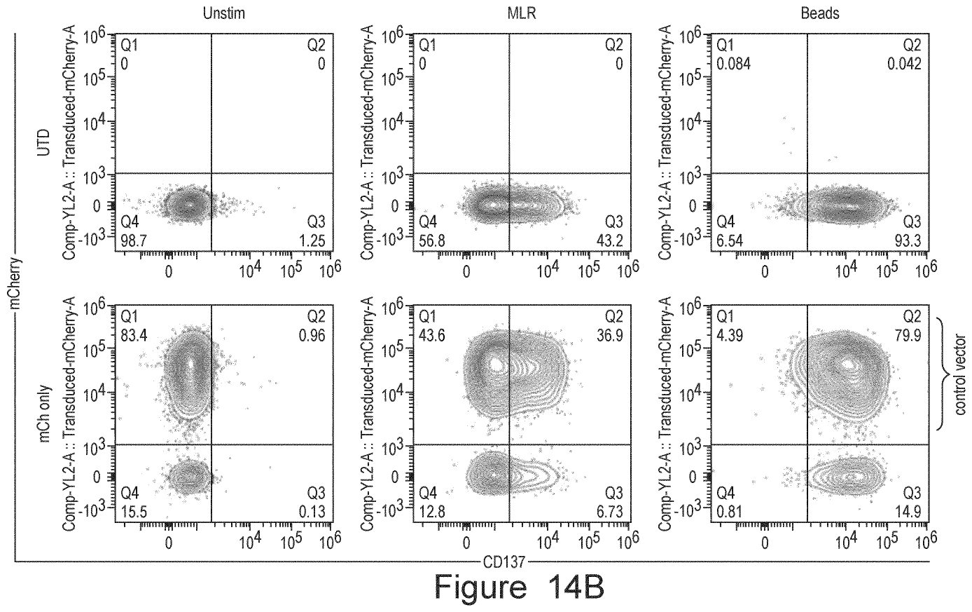

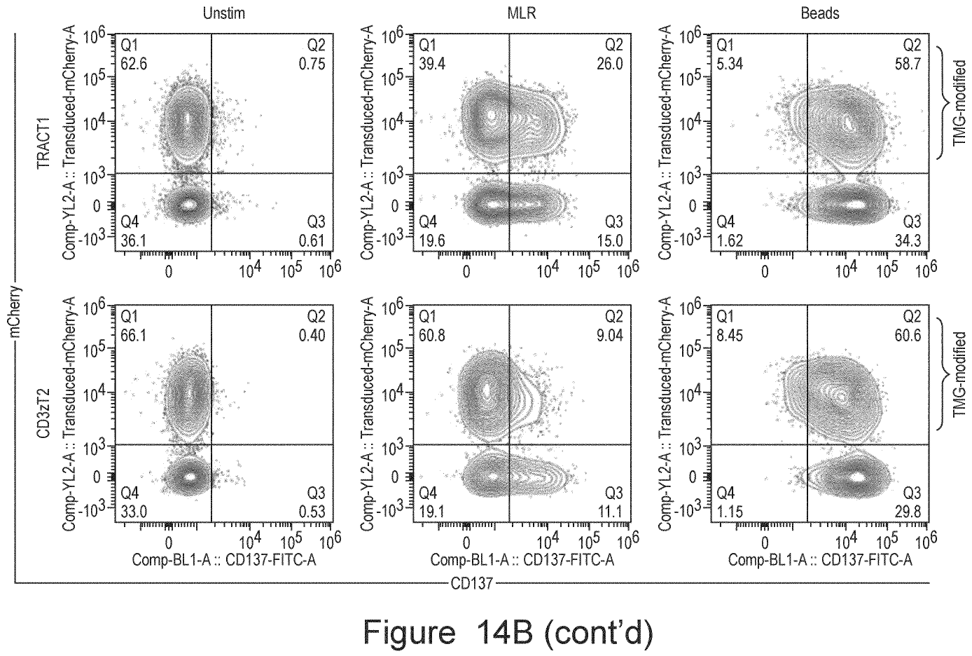

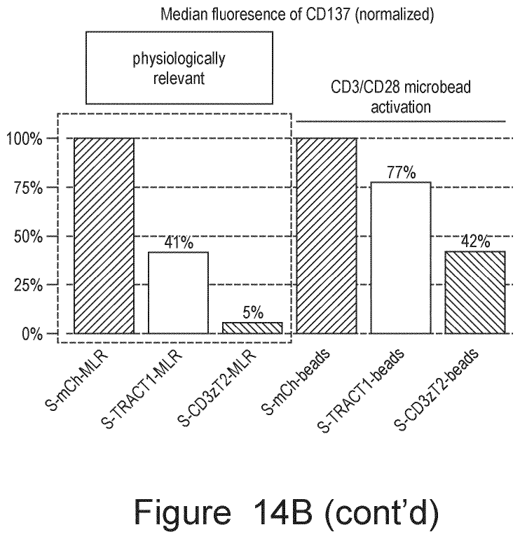

Next, mixed lymphocyte reactions were performed using two miRNA constructs to assess if they would infer a loss of alloreactivity when co-cultured with unmatched T-cells. In the first case, PBMCs were transduced with a miRNA against CD3z_T2. Second, a miRNA against TRAC_T1 was used to achieve intermediate gene silencing of the TCR. Upon lentivector transduction, T-cells were generated with 95% and 30% TCR silencing using the previously mentioned constructs (FIG. 14A). After allowing these TCR-deficient T-cells to come to resting state (12 days post activation), the cells were co-cultured with irradiated stimulator T-cells at a ratio of 1:1. At 24 hours post co-culture, cells were harvested and assessed the gene-modified CD8+ T-cells (mCherry positive) for expression of the CD137 activation marker. A 60% decrease in CD137 was observed in T-cells transduced with a construct against TRAC_T1 target sequence. This was a notable decrease for these T-cells having 30% TCR silencing. Further, in CD8+ T-cells with 95% TCR silencing (miRNA against CD3z_T2), negligible CD137 expression was observed (FIG. 14B). This confirmed that TCR-deficient T-cells, including those silenced with the CD3z_T2 construct, were not alloreactive against genetically unmatched stimulator T-cells.

In conclusion, target sequences against CD3z were identified as being efficient at TCR silencing. Some differences were observed when using two and three hairpin constructs with this same sequence. Moreover, CD8+ T-cells modified with a single hairpin miRNA construct showed negligible alloreactivity in mixed lymphocyte reactions.

### Results, Conclusions and In Vivo Studies

Novel gene silencing constructs against TRAC and CD3z were developed, determined as high performers based on TCR silencing efficiency and selected for deep characterization. The constructs were delivered to primary T-cells via lentiviral vector transduction, followed by expansion in G-Rex cell culture plates. Gene-modified T-cells were purified via depletion of TCR-expressing cells and assessed for loss of alloreactivity in mixed lymphocyte reactions (MLRs). In summary, functional silencing of the TCR and the successful development of non-alloreactive T-cells have been demonstrated.

### Example 4: HLA-II Down-Regulation Via CIITA Silencing

Class II transactivator (CIITA) is a transcription factor essential for transcriptional activity of the human leukocyte antigen (HLA) class II promoter. CIITA acts in a coactivator-like fashion through protein-protein interactions bringing together factors binding to the proximal HLA class II promoter and the transcription machinery. It may also activate HLA class II transcription by modifying proteins that bind to the promoter. The promoter element requirements for CIITA-mediated transcription are distinct from those of constitutive HLA class I transcription.

CIITA is expressed and regulates the expression of HLA class II molecules in human activated T-cells, thus controlling the response to foreign antigens and the maintenance of tolerance. CIITA expression is upregulated in response to inflammatory stimuli. In vitro, T-cell malignancies exhibit CIITA-dependent HLA class II—deficient phenotype, hence preventing cell death (PMID: 11207239).

In the context of engineered allogeneic cell therapies, engineered T-cell therapies, and particularly allogeneic chimeric antigen receptor (CAR) T-cells, HLA class II expression is upregulated in activated CAR T-cells and hence prone to rejection by CD4+ T-cells of the host's immune system. Gene silencing of HLA class II is thus a possible solution to limit this rejection. Additionally, a construct silencing HLA-I could also be used for creating universal donor cell therapies from various sources, including induced pluripotent stem cells (iPSCs).

### Gene Silencing in Target Cells

Target sequences were identified and prioritised for screening. Target sequences were identified in respect of human CIITA (ENSEMBL: ENSG00000179583). Where necessary with identified target sequences, the first nucleotide of the guide strand can be changed, e.g. from a cytidine (C), to thymine (T), in order to promote guide strand incorporation into the RISC (RNA-induced silencing complex).

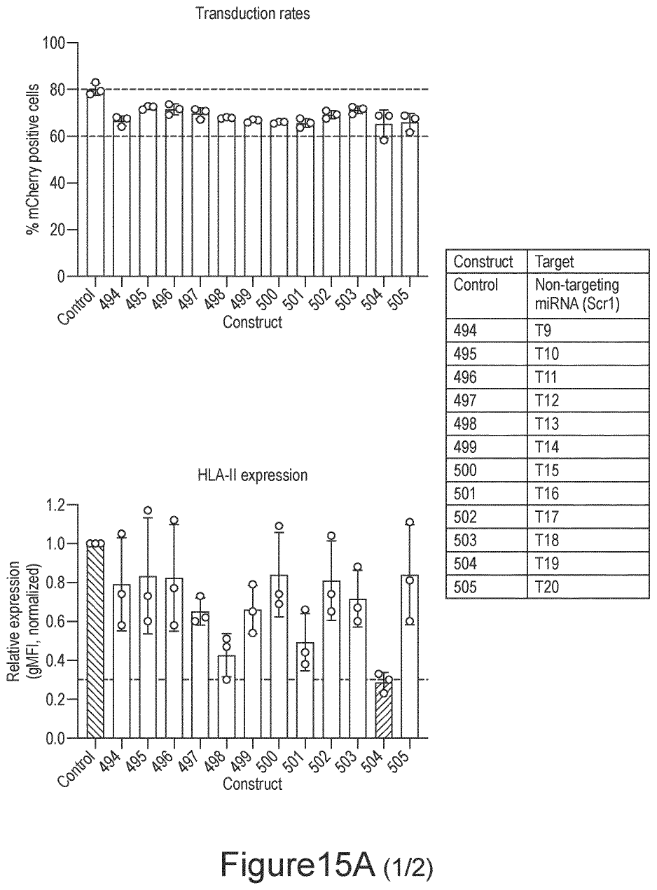

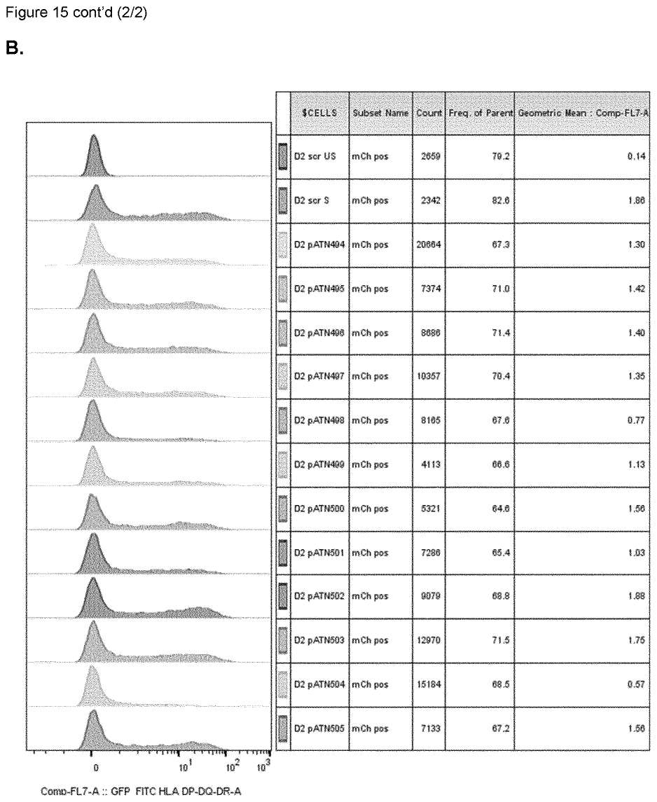

Screening experiments with target sequences designed to silence CIITA were carried out in primary T-cells, for which the data is shown in FIG. 15. From this second screen, three constructs (pATN498, pATN501 and pATN504) resulted in evident downregulation of HLA class II. In particular, pATN504 achieved consistent HLA class II silencing over a range of 65-75% across three donor T-cell products.

### Conclusion

Novel miRNA gene constructs capable of silencing CIITA were screened for and successfully identified. This translated to highly efficient silencing of HLA class II cell surface expression. The target sequence CIITA_T19 (in construct pATN504) performed most efficiently and consistently across three different T-cell donor products.

### Example 5: Bimodal Constructs for Simultaneous HLA-I and TCR Down-Regulation

The present inventors developed a novel bimodal gene construct for simultaneous CAR expression and microRNA-mediated gene silencing (miCAR), which not only facilitates highly efficient multiplex gene silencing, but also “tunable” silencing of target genes. Using this approach, the present inventors developed allogeneic CAR T-cells with simultaneous CAR expression and functional silencing of the TCR and HLA-I. More specifically, this approach was able to completely silence TCR expression, while optimizing the level of HLA-I silencing to strike a balance between immune rejection by both CD8+ T-cells and NK cells.

Although ablation of HLA-I expression on graft CAR T-cells may protect against rejection by host CD8+ T-cells, it conversely renders the cells prone to NK cell rejection. One solution to overcome this is to additionally co-express an inhibitory molecule of NK cells, typically a non-classical HLA-I molecule, such as HLA-E, HLA-G or HLA-F. It has also been shown that CD47 co-expression protects from NK cell mediated rejection. An alternative solution is to silence HLA-I expression to a range that not only protects graft cells from host CD8+ T-cells, but also allows for sufficient protection against host NK cells, without the need for co-expression of additional receptors to avert NK cell mediated rejection.

### Methods

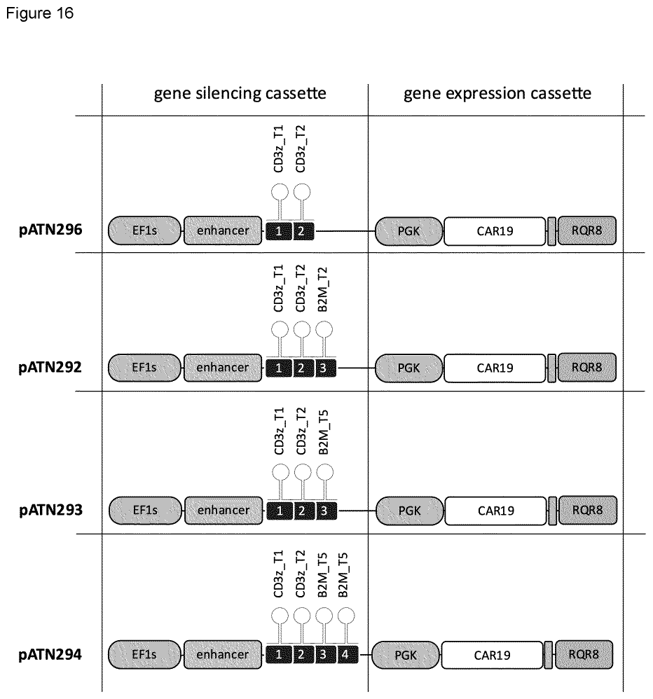

Gene constructs were first created to silence HLA-I to varying levels, each of which were cloned into a previously optimized miCAR construct expressing an anti-CD19 CAR (CAR19) and miRNA that silences TCR expression with high efficiency (a first miRNA hairpin targeting CD3z_T1 and a second miRNA hairpin targeting CD3z_T2). A schematic overview of some of the constructs created is presented in FIG. 16.

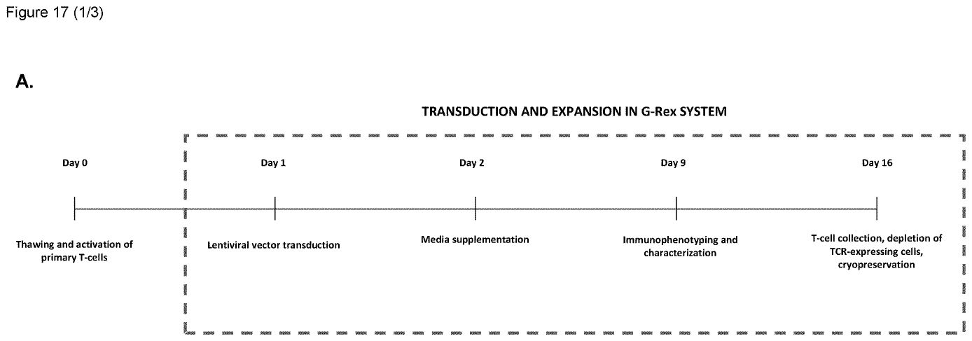

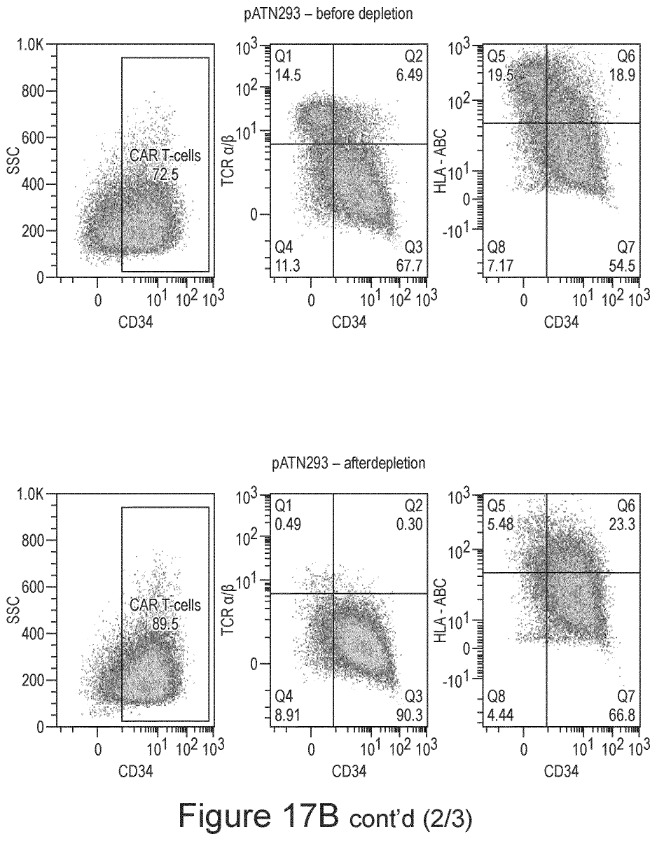

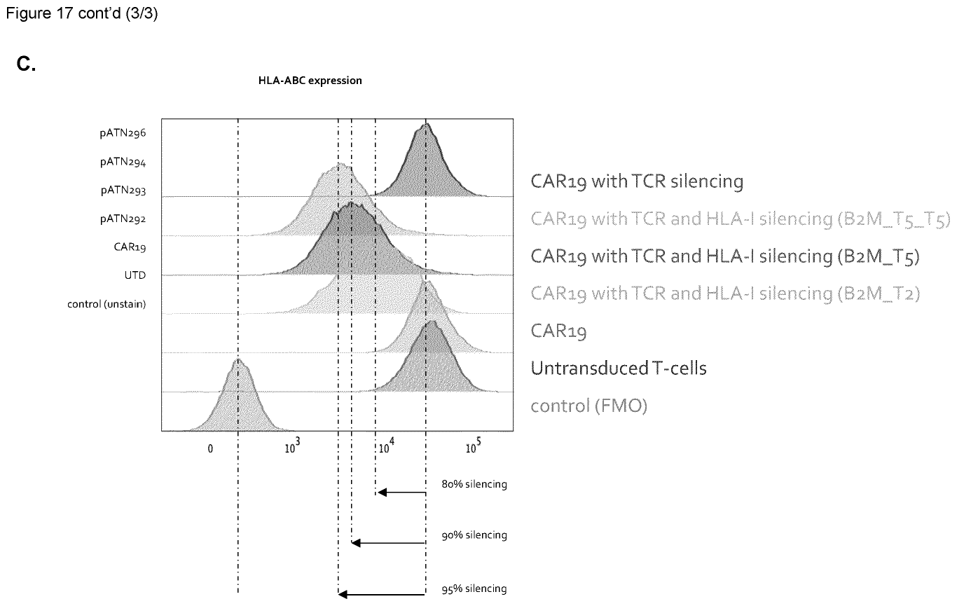

Primary T-cells were modified via lentiviral vector transduction, expanded in G-Rex cell culture plates, and purified by depletion of TCR-expressing cells. In vitro characterization included FACS immunophenotyping, cytotoxicity of CD19-expressing cells, and hypoimmunogenicity testing with unmatched T-cells and NK cells in mixed lymphocyte reactions (MLRs). FIG. 17 illustrates this process and its results.

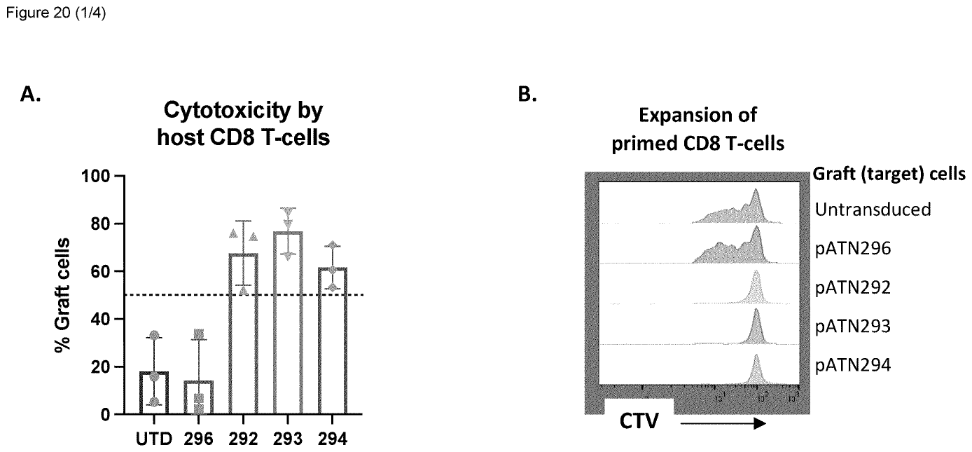

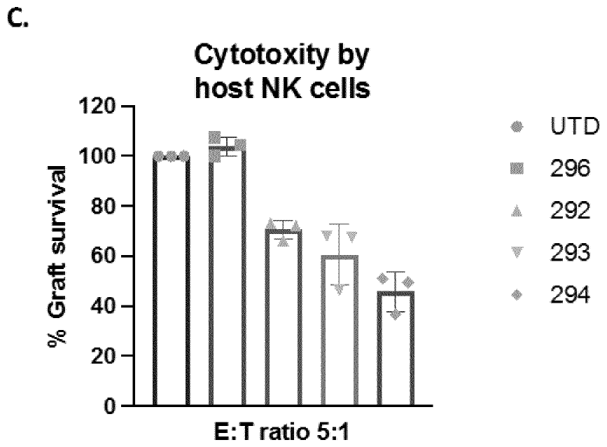

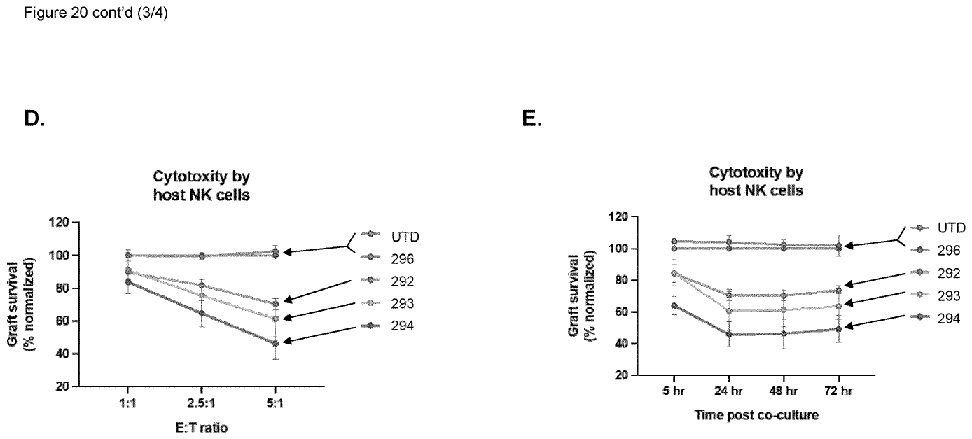

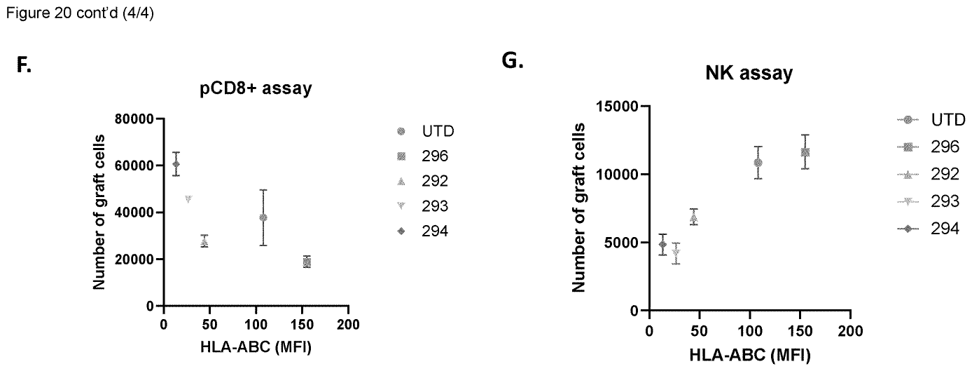

For mixed lymphocyte reactions with unmatched CD8+ T-cells and NK cells using allogeneic and hypoimmunogenic miCAR19 T-cells (as shown in FIG. 20F), host PBMCs were primed with mitomycin treated graft donor cells (CAR19 T-cells), after which CD8 positive T-cells were isolated and labelled with CellTrace Violet (CTV) dye. Primed CD8+ T-cells (effector, E) were then co-cultured with graft miCAR19 T-cells (target cells, T) at an E:T ratio of 1:1. Six days post plating the co-culture, cells were analyzed by means of flow cytometry. Similarly, for the MLRs with Nk cells (FIG. 20G), host NK cells (effector cells, E) were co-cultured with graft miCAR19 T-cells (target cells, T) at an E:T ratio of 5:1. After 48 hours, cells were analyzed by means of flow cytometry to assess for the proportions of NK cells and T-cells based on CD56 and CD5 expression, respectively.

### Results

Multiplex engineered, allogeneic miCAR19 T-cells were successfully created, all of which were completely silenced for TCR and with tuned silencing of HLA-I over a range of 70-90% (FIG. 17). While CAR functionality was maintained in recursive cytotoxic assays against tumor cells, the developed novel miCAR 19 T-cells were also protected from both CD8+ T-cell and NK cell mediated cytotoxicity in MLR assays, with rejection of CD19 CAR T cells by primed CD8 T or

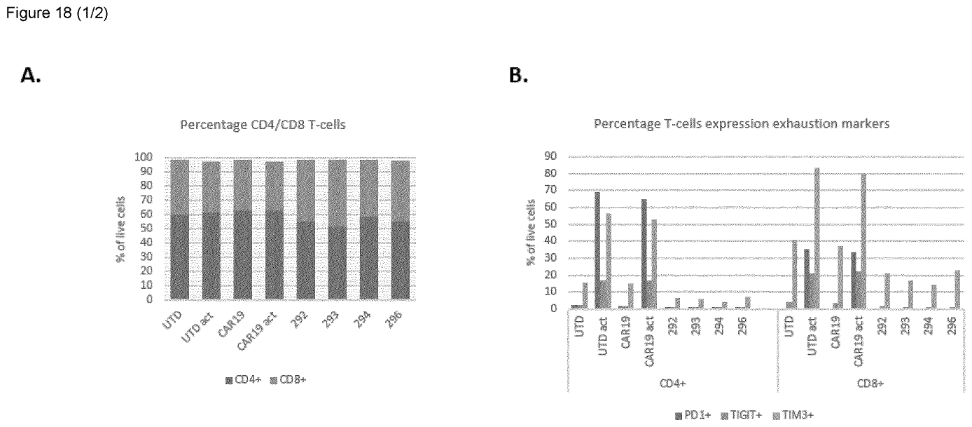

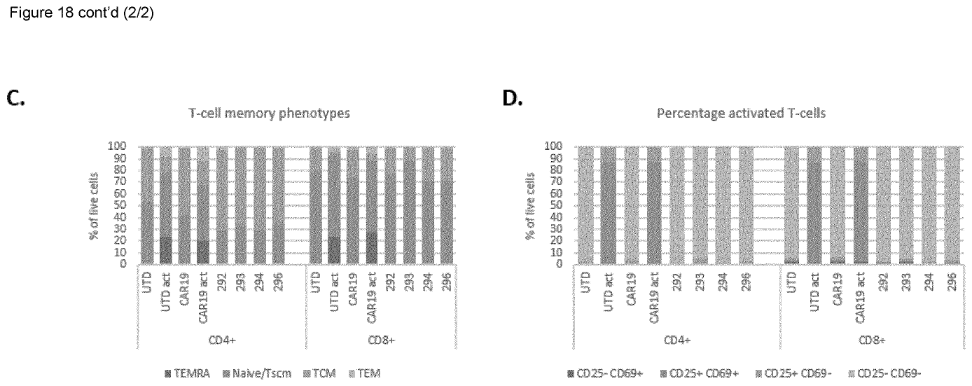

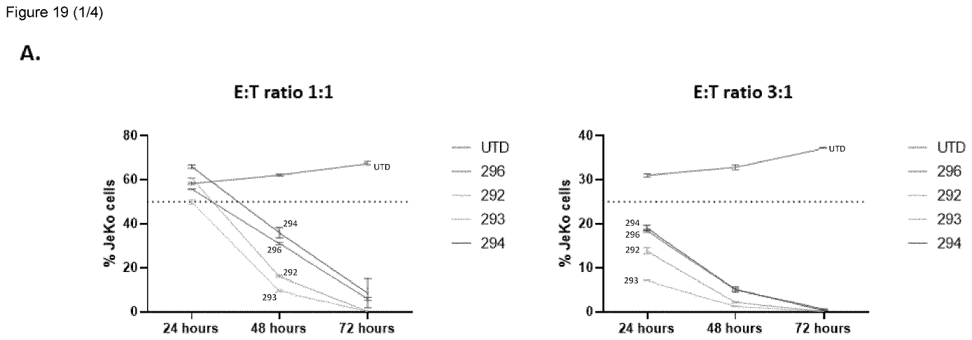

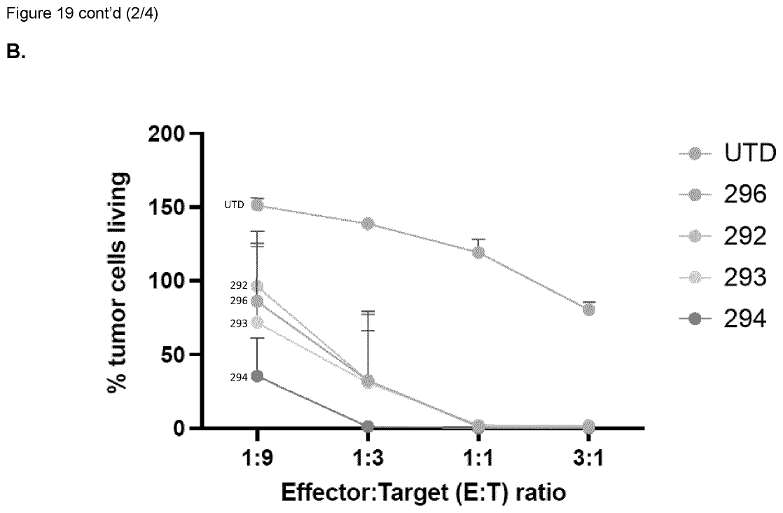

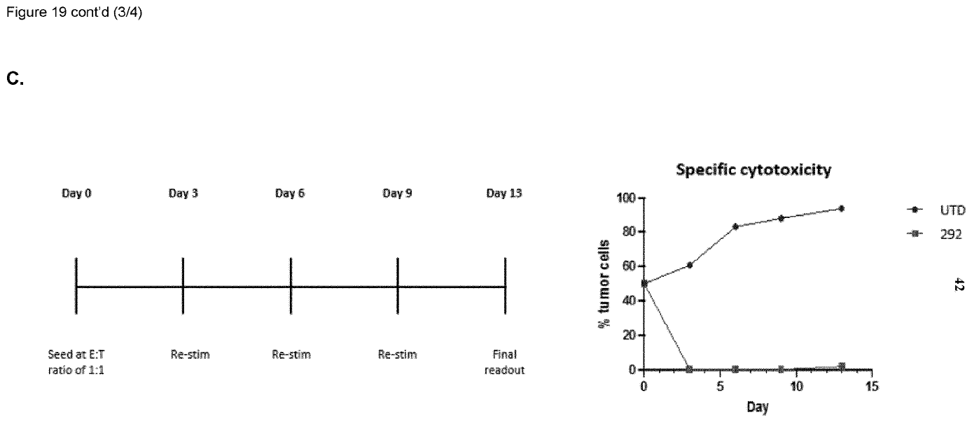

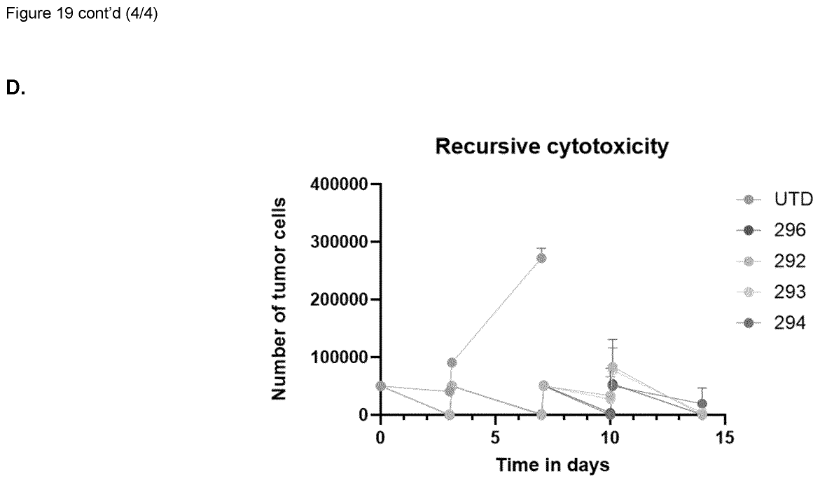

NK cells also correlated with HLA-ABC levels, in vitro (FIGS. 18, 19, and 20). Thus, an efficient approach to multiplex engineering of allogeneic and hypoimmunogenic CAR T-cells, with functional silencing of the TCR and HLA-I, has been demonstrated.

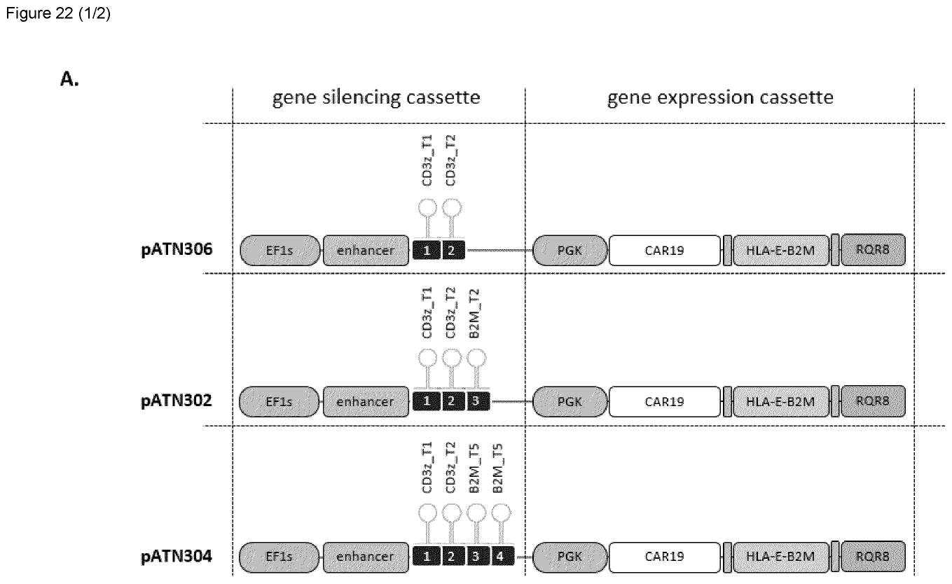

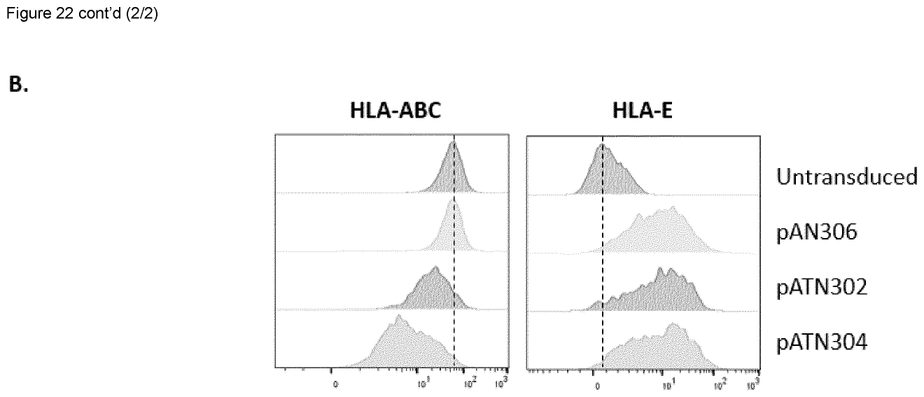

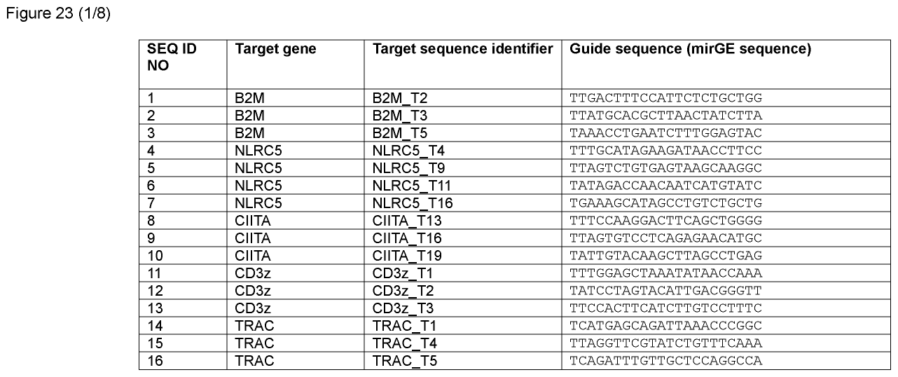

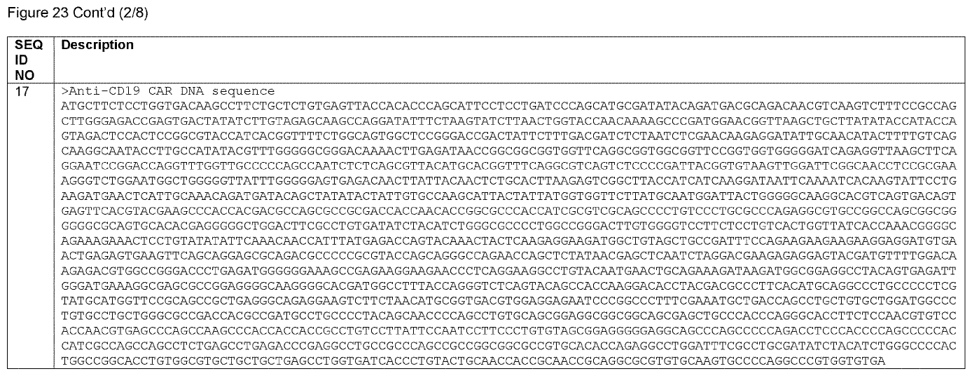

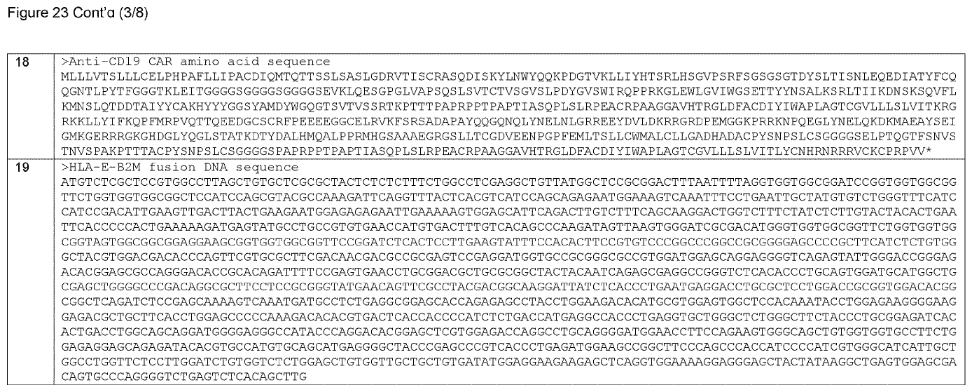

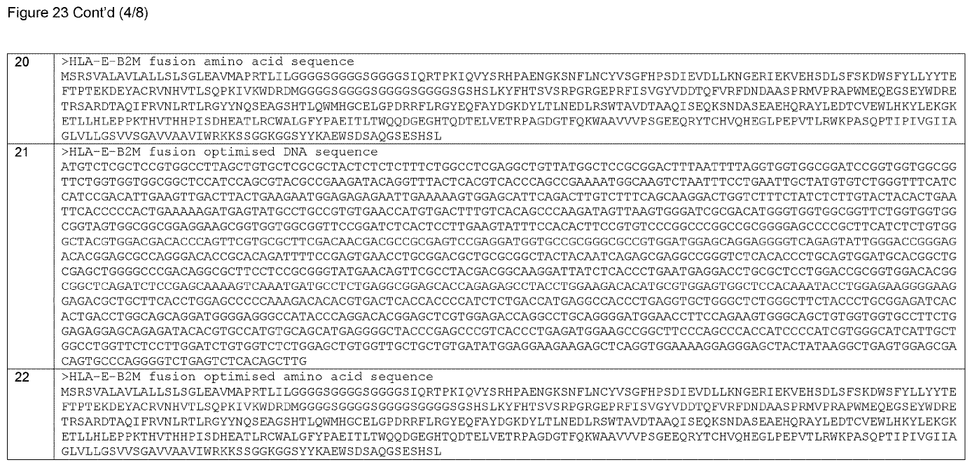

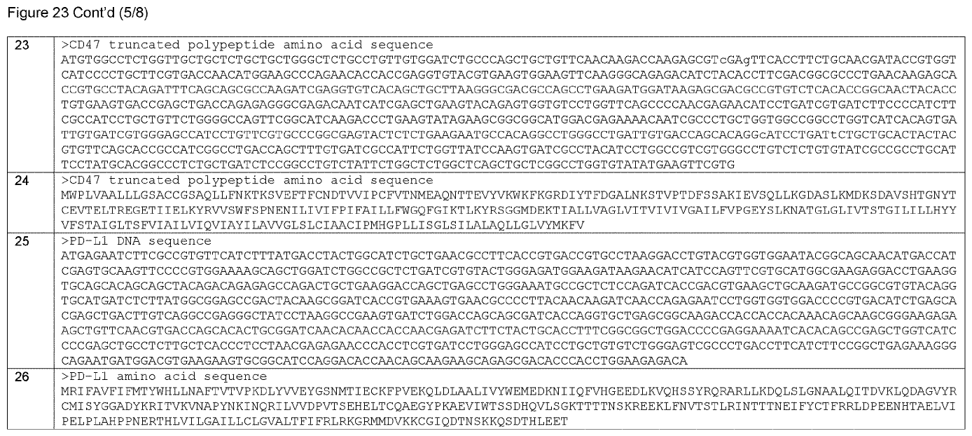

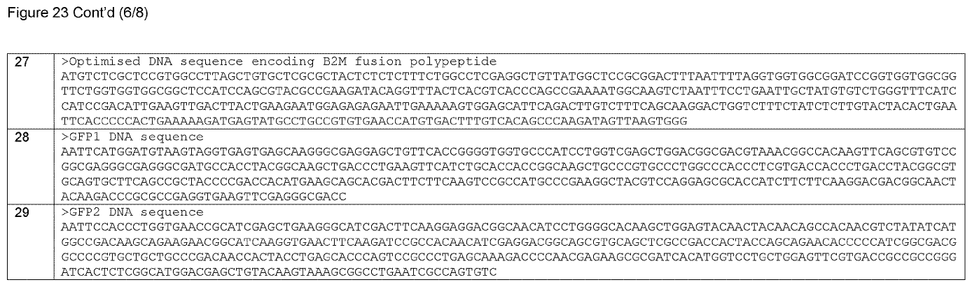

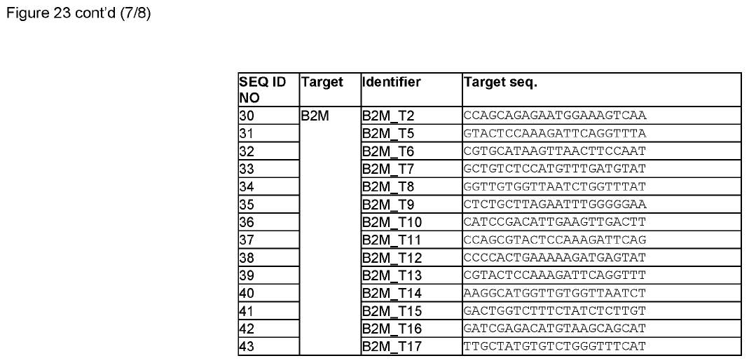

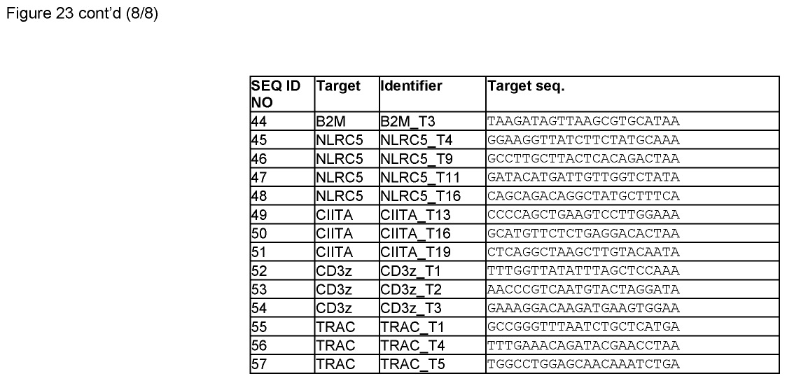

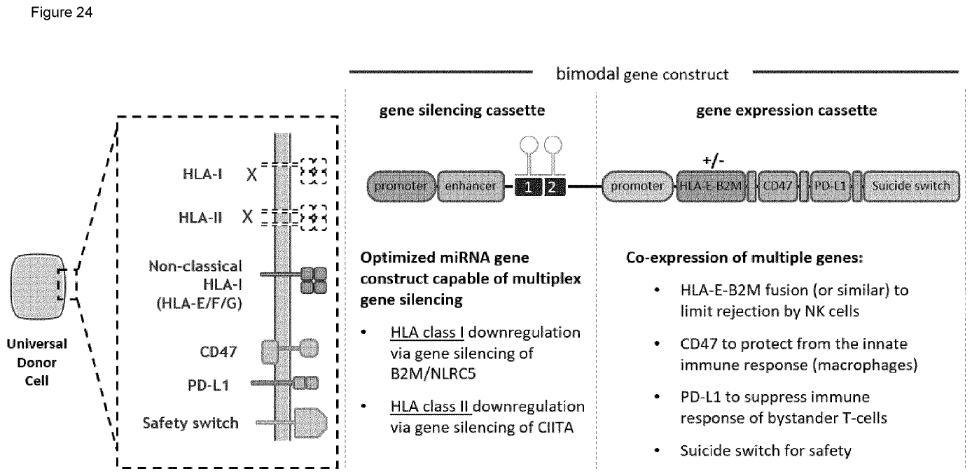

Moreover, a complementary approach of efficient HLA-I knockdown to avoid CD8+ T-cell killing combined with non-classical HLA-B2M fusion protein expression to avoid NK cell killing was validated (constructs shown in FIG. 22). Approximately 60-90% silencing of HLA-ABC (classical HLA-I) was achieved from the use of miRNA constructs targeting B2M, whilst HLA-E expression was maintained (FIG. 22).

### Example 6: Further Characterising of Cells with Simultaneous HLA-I and TCR Down-Regulation

Allogeneic CAR T-cells with simultaneous CAR expression and functional silencing of the TCR and HLA-I were further characterised in view of their response to CD3 stimulation, and separately to IL-3 or IL-15 cytokines.

### Methods

The same gene constructs as in Example 5 were used to silence HLA-I to varying levels, each of which were cloned into a previously optimized miCAR construct expressing an anti-CD19 CAR (CAR19) and miRNA that silences TCR expression with high efficiency (a first miRNA hairpin targeting CD3z_T1 and a second miRNA hairpin targeting CD3z_T2).

CD3 stimulation assay: Engineered CAR T-cells were stimulated with anti-CD3 antibody (OKT3) over a concentration range of 0-17.5 ug/mL, and 24 hours later assessed for expression levels of the CD137/CD69 activation markers.

Cytokine outgrowth assay: Engineered CAR T-cells were cultured either with or without IL-7 and IL-15 over a period of 13 days. Every 3-4 days, cells were counted, and dead cells excluded by using Trypan Blue.

### Results

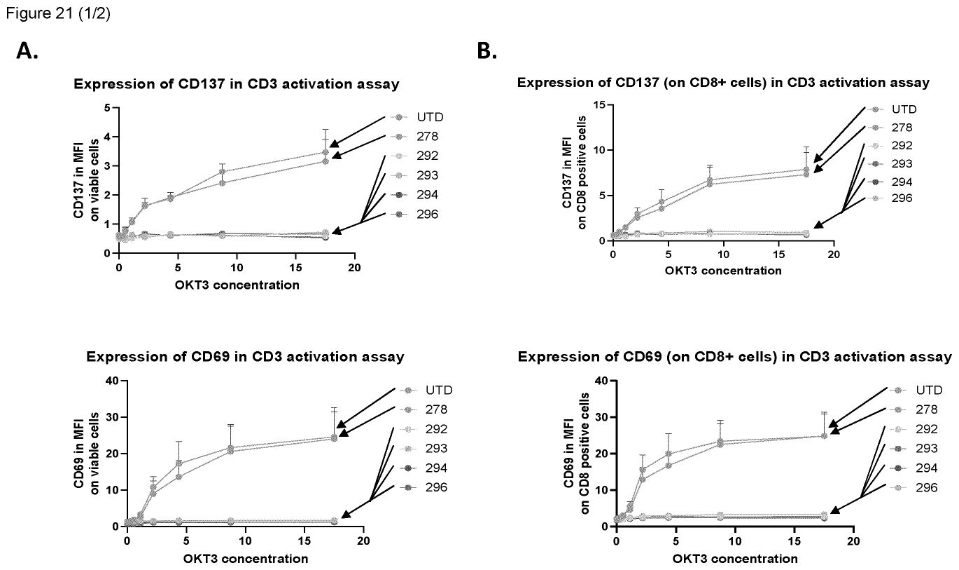

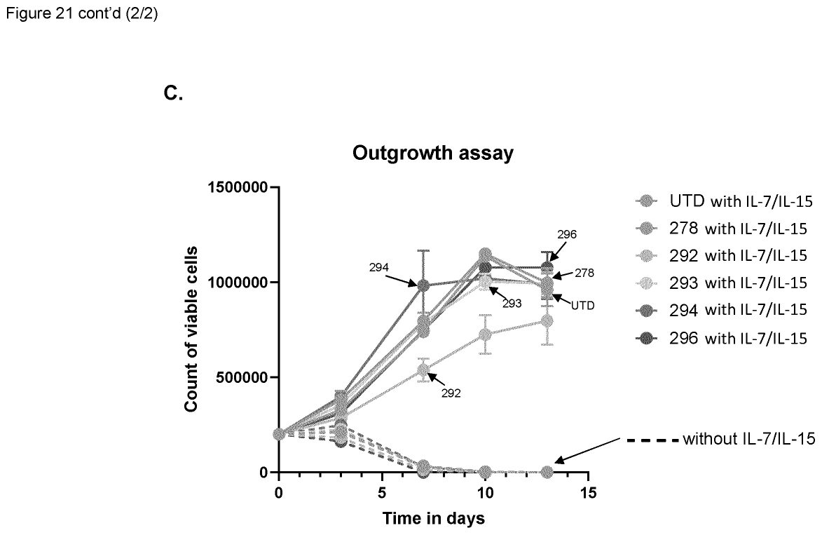

CD3 stimulation assay: Untransduced T-cells and control CAR T-cells (278) were shown to be activated by OKT3 from a concentration of 0.54 ug/mL and increased equivalently, as shown in FIGS. 21A and 21B. Notably, activation marker expression remained unchanged over this same concentration range for TCR-silenced CAR T-cell populations, confirming the loss of TCR functionality upon silencing of the receptor.

Cytokine outgrowth assay: Cell survival was observed in all cell populations in the presence of cytokines, while no outgrowth was reported for cells in their absence, as shown in FIG. 21C.

### Example 7: Preclinical Scale Production of TCR-Silenced T-Cells

### Methods

Primary T-cells were activated with TransAct (Miltenyi Biotec) according to the manufacturer's instructions and transduced two days later with lentiviral vectors carrying miRNA gene constructs for silencing of CD3z expression. Following transduction, T-cells were seeded in G-Rex cell culture plates and expanded for seven days in TexMACS medium with IL-7 and IL-15, after which the cells were harvested and analysed via flow cytometry.

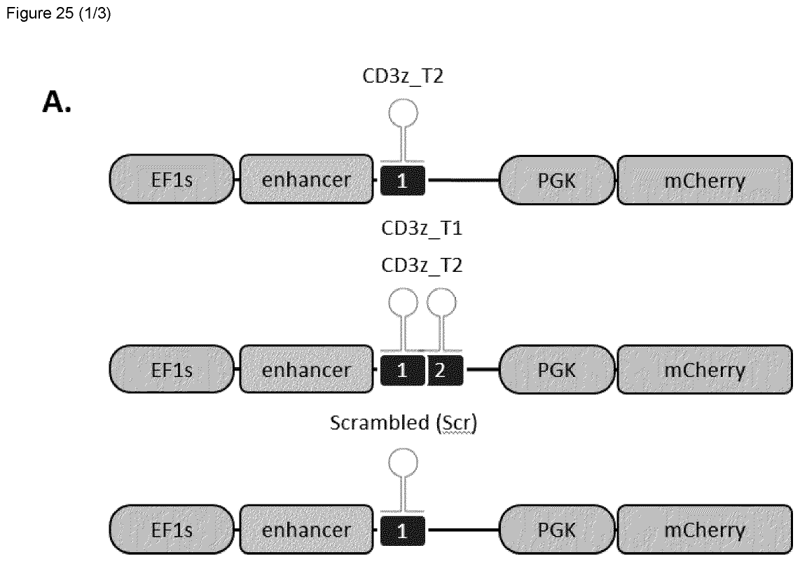

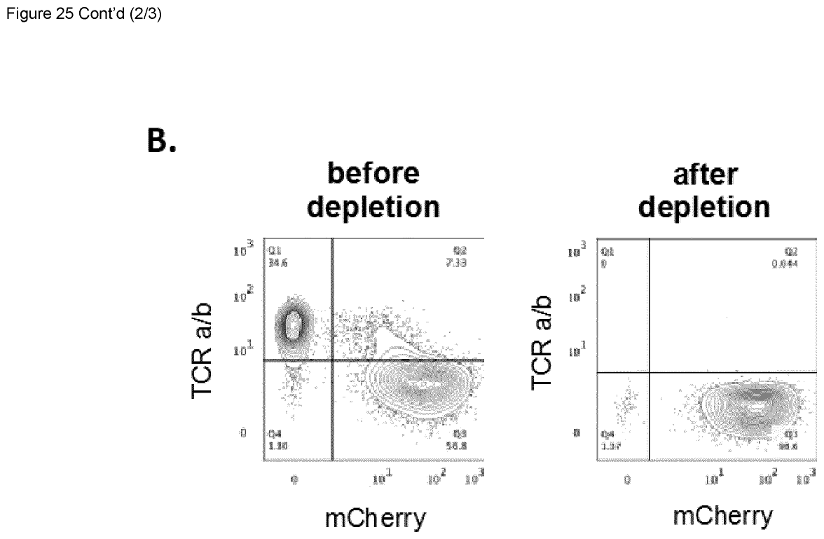

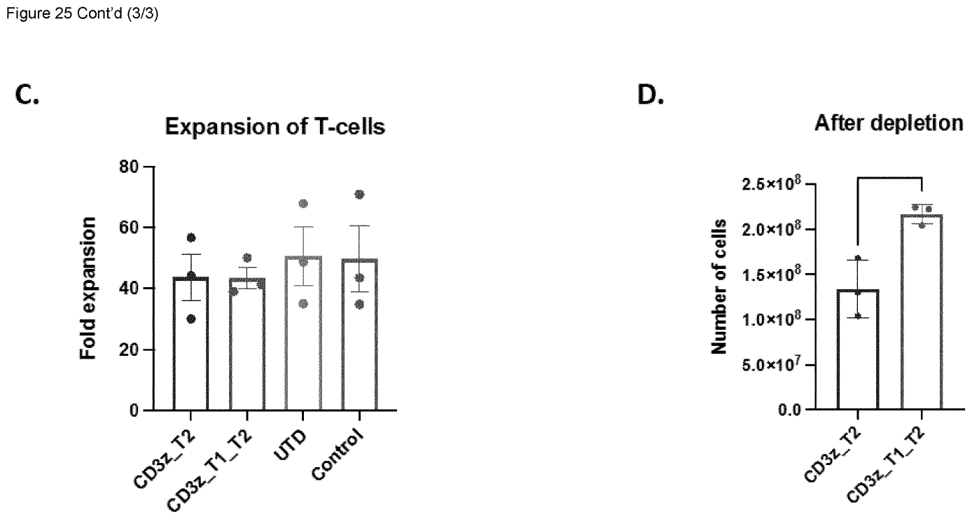

As shown in FIG. 25A. Three constructs were used in this experiment, namely (i) a single hairpin (1 hp) miRNA targeting CD3z (T2), (ii) a dual-hairpin (2 hp) miRNA targeting two different regions of the CD3z transcript (T1_T2), and (iii) a non-targeting miRNA (with scrambled guide strand sequence).

### Results

As shown in FIG. 25B, cells from n=3 donors for each construct were transduced in excess of 65% (assessed by mCherry positivity). Following depletion of the remaining TCR positive cells in conditions transduced with constructs to silence TCR, a pure population of TCR-mCh+ cells remained.

As shown in FIG. 25C, on average, 40-50 fold expansion of T-cells was observed over the 9 days of production, with no statistically significant differences in fold expansion being reported across the batches (Kruskal-Wallis ANOVA, p=0.975). D. Notably, although equivalent numbers of cells were harvested for each condition, the yield of TCR-silenced T-cells (engineered from the dual-miRNA CD3z_T1_T2) was nearly double when compared to TCR-silenced cells engineered from the single miRNA gene construct targeting CD3z_T2 (Unpaired T-test, p=0.0136). Thus, the miRNA constructs of the present invention that comprise two miRNA hairpins down-regulating TCR, and the cells comprising those constructs, are capable of providing an improved yield of TCR-silenced cells.

### Example 8: Generation of Universal Donor Cells

Induced pluripotent stem cells (iPSCs) can be used for the generation of universal donor cells (UDCs). Thus, UDC constructs were used to silence and over-express respective targets as shown in Table 4, in iPSCs. Further validation for cells expressing HLA-II molecules was then conducted using T-cells transduced with a UDC construct.

TABLE 4

| Construct | | | | | | |
|---|---|---|---|---|---|---|
| number | Silencing | | Over-expression | | | |
| 1692 | β2M_T5 | CIITA_T19 | CD47 | PDL1 | RQR8 | |
| 1689 | β2M_T5 | CIITA_T19 | CD47 | PDL1 | RQR8 | β2M- |
| | | | | | | HLAE |
| 1690 | scr48 | | CD47 | PDL1 | RQR8 | β2M- |
| | | | | | | HLAE |

### Methods

iPSCs transduced with UDC constructs: iPSCs were plated in mTeSR complete medium containing CloneR2 (StemCell technologies). The same day, after they attached, cells were transduced by adding lentiviruses directly to the medium (MOI 5). The medium was exchanged for fresh medium after 24 h. FACS was performed 6 days after transduction to assess for protein overexpression and silencing of targets of interest.

T-cells transduced with UDC constructs: Cryopreserved T-cells were thawed and activated using CD3/CD28 Dynabeads at a ratio of 3:1. Activated T-cells were transduced 24 hours later with lentiviral vectors carrying the miRNA gene silencing constructs. Transductions were performed in high density volumes (2 million cells per mL), and the medium replenished after 18-24 hours and every other day thereafter for T-cell maintenance at a cell density of 1 million/mL.

### Results

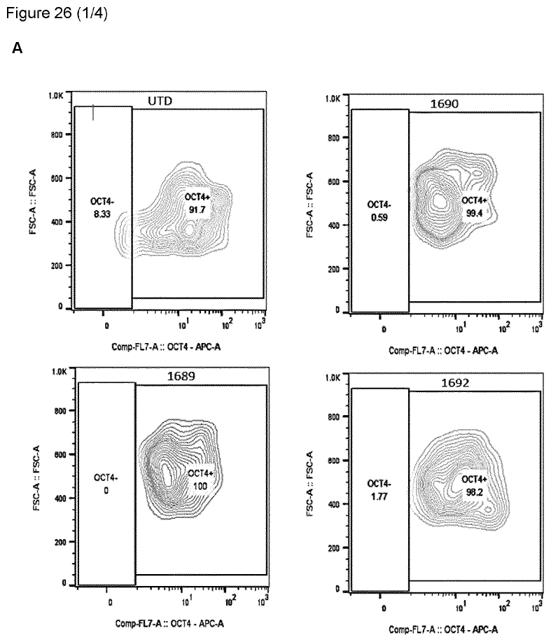

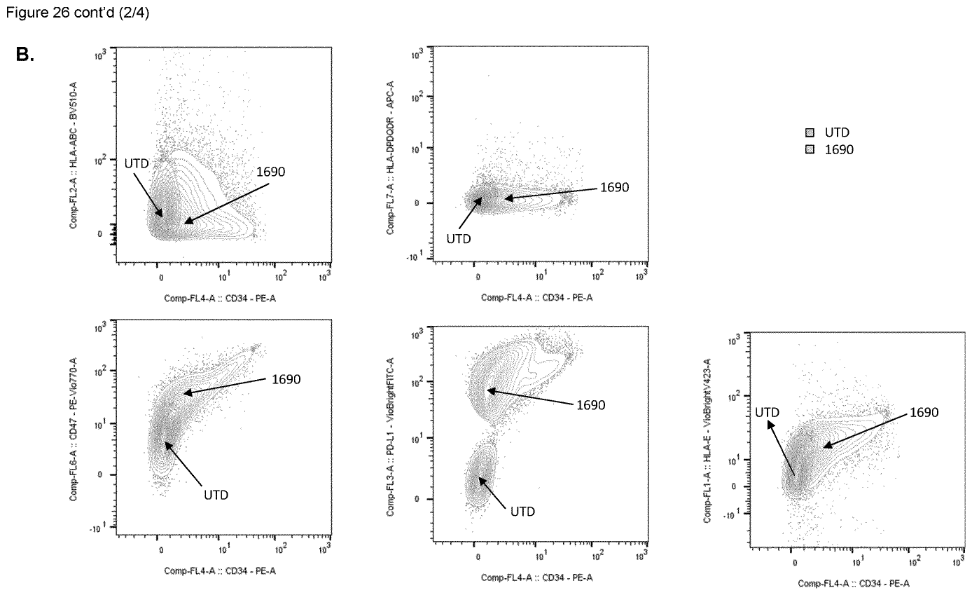

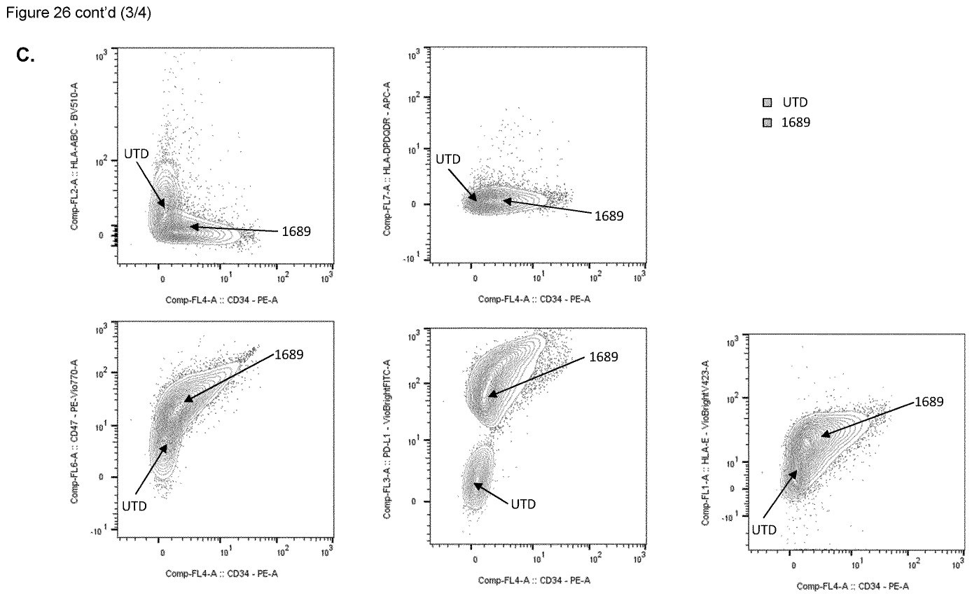

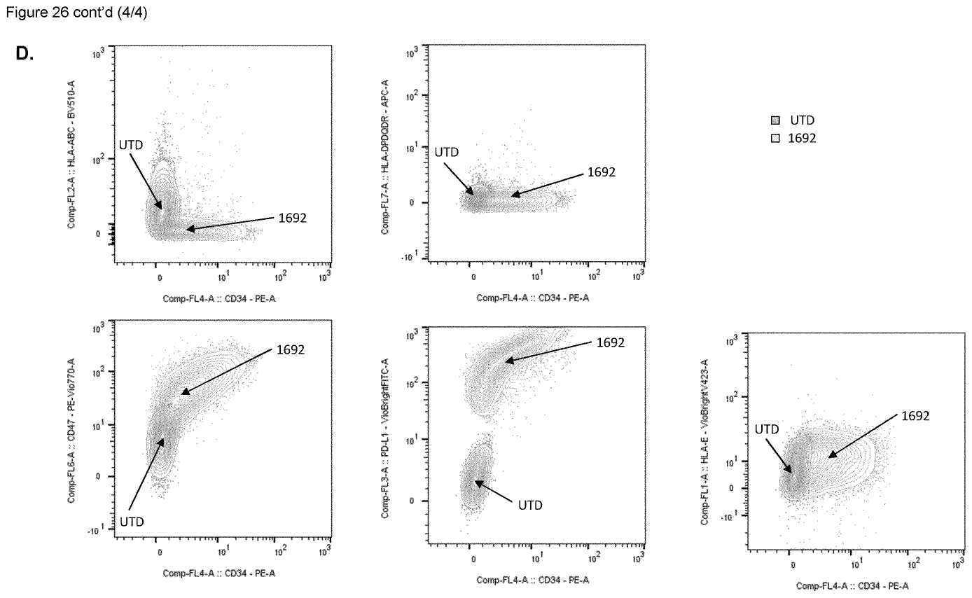

The iPSCs used all showed >90% expression of OCT4, which is associated with an undifferentiated phenotype, confirming that the cells used present a stem cell phenotype (FIG. 26A). iPSCs can be transduced with a UDC construct that leads to the overexpression of CD47, PD-L1 and HLA-E, as well as CD34 (FIG. 26B). In addition, iPSCs can also be transduced with a UDC construct that allows concomitant silencing of HLA-ABC and CD47, PD-L1, HLA-E and CD34 overexpression (FIG. 26C). Since HLA-E overexpression might not be mandatory for the generation of universal donor cells, it is also shown that iPSCs can be transduced with a UDC construct that allow for overexpression of CD47, PD-L1 and CD34, as well as silencing of HLA-ABC (FIG. 26D).

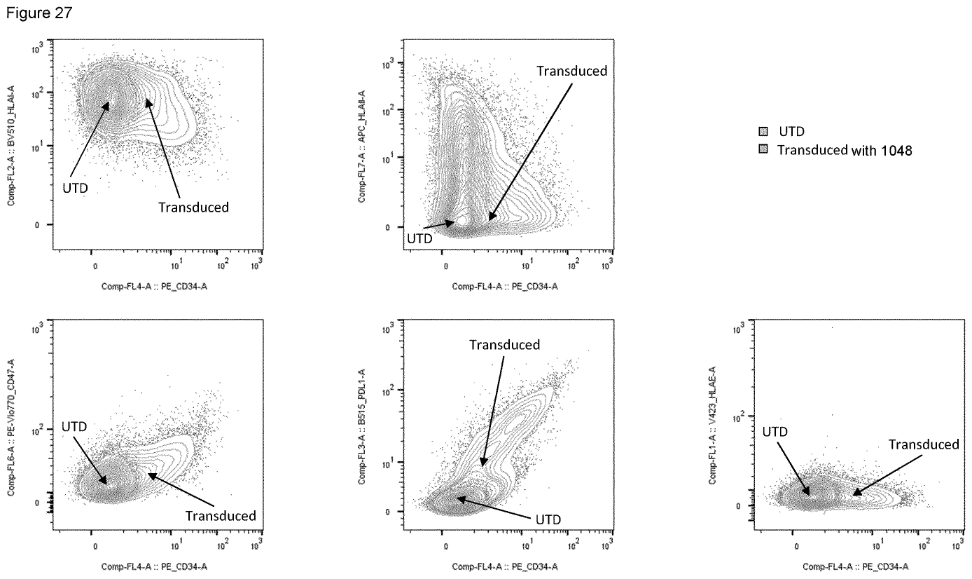

As iPSCs do not express HLA-II molecules, T-cells-being a more differentiated cell type that does express HLA-II-were also transduced with the UDC construct, proving that the constructs lead to both HLA-I and HLA-II silencing, and at the same time overexpression of CD47, PD-L1 and CD34 (FIG. 27).

Thus, in a preferred embodiment of the universal donor cell aspects of the present invention, B2M and CIITA are downregulated, preferably by miRNA, preferably by B2M_T5 CIITA_T19 or miRNA sequences defined with reference thereto herein. In a further preferred embodiment, CD47, PDL1 and RQR8 are upregulated or overexpressed. In a further preferred embodiment, β2M-HLAE is upregulated or overexpressed.

### Example 9: In Vivo Study of TCR-Silenced Cells in Immunodeficient Mice

For in vivo studies, immunodeficient mice were irradiated prior to infusion of 20 million TCR-silenced cells and assessed over 100 days for GvHD onset. Novel miRNAs resulting in >50% TCR silencing were identified, which were multiplexed in various combinations for selection of optimal constructs. Depletion of TCR-expressing cells resulted in a >99% pure population of gene-modified, TCR negative T-cells as evidenced by co-expression of mCherry reporter gene. In MLR assays, negligible activation of TCR-silenced cells was observed. When infused in immunodeficient mice, control-transduced T-cells resulted in substantial weight loss and GvHD onset, with less than 50% of mice surviving by day 58. However, mice receiving TCR-silenced T-cells remained healthy with all surviving to day 100 (data not shown). In summary, functional silencing of the TCR and the successful development of non-alloreactive T-cells have been demonstrated.

### Methods

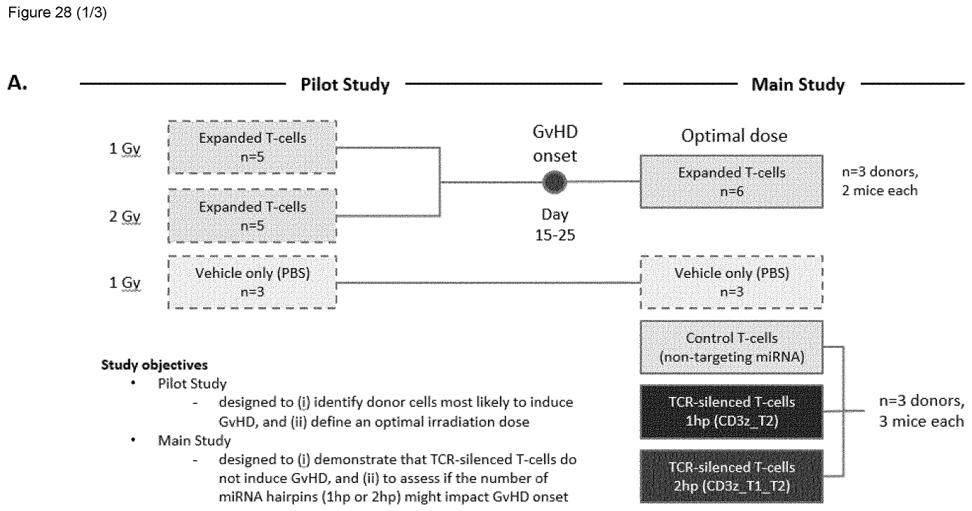

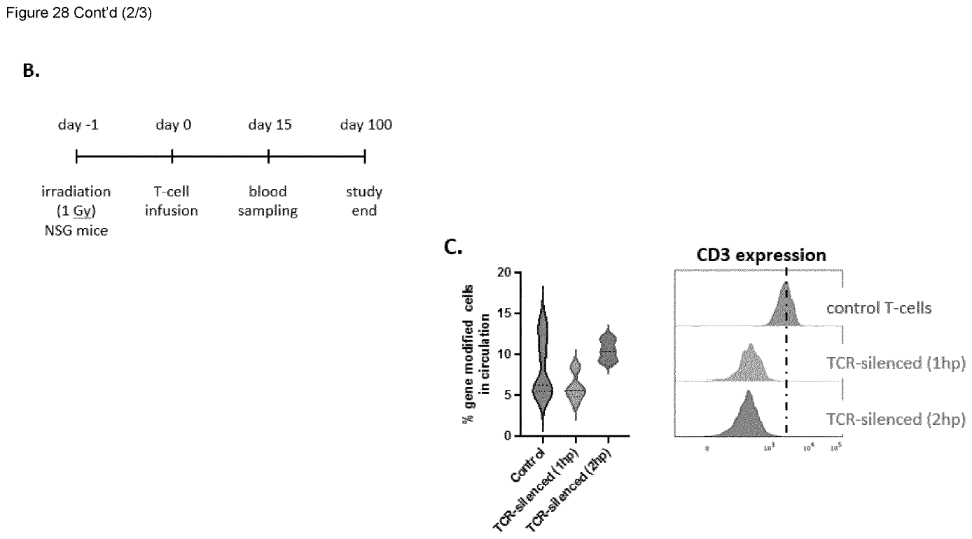

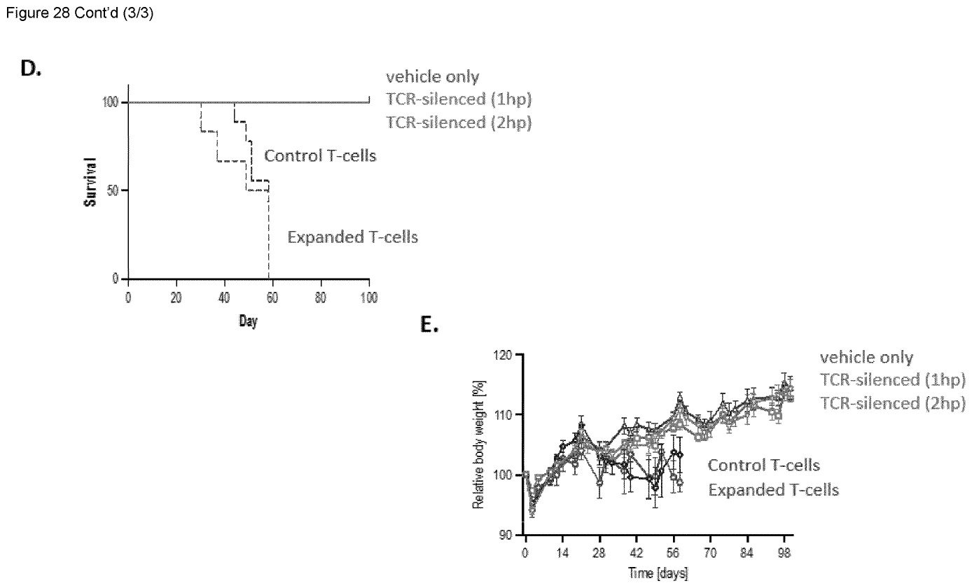

NOD SCID gamma (NSG) mice were infused with TCR silenced T-cells to assess if these cells remain non-alloreactive in vivo, and thus do not result in the onset of graft vs host disease (GvHD). FIG. 28A illustrates the overall study design, including pilot and main studies. FIG. 28B illustrates the main study design, where NSG mice were irradiated with a 1 Gy dose, infused with 20 million T-cells 24 hours later, and followed up over 100 days.

### Results

As shown in FIG. 28C, on day 15, blood was collected for flow cytometric analysis to confirm engraftment of modified cells (based on mCherry reporter gene expression). As indicated, >5% of cells in circulation were gene modified, confirming T-cell engraftment. Moreover, sustained TCR/CD3 silencing was shown in mice receiving T-cells gene modified with CD3z-targeting miRNA. As shown in FIG. 28D, survival curves illustrated complete survival of mice receiving TCR silenced T-cells through 100 days (irrespective of whether the cells were gene modified with 1 hp or 2 hp miRNAs against CD3z). Notably all mice dosed with control-transduced or expanded T-cells died due to GvHD onset by day 58. As shown in FIG. 28E, correspondingly, the relative body weight percentages of the latter mice started to trend downwards from approximately day 14, while mice receiving vehicle only or TCR silenced T-cells continued to gain weight over the 100 day study period.

### Example 10: In Vivo Study of “Tuned” HLA-I and TCR/CD3-Silenced Cell Persistence in Immunodeficient Mice

Immunodeficient mice were irradiated prior to infusion of host T-cells and Raji-luc cells followed by “tuned” HLA-I and TCR/CD3-silenced cells at one of three levels (no HLA-I silencing, 80% and 90% silencing) and assessed over 32 days for detectable “tuned” silencing of HLA-I. The “tuned” HLA-I silencing was clearly detectable via flow cytometry after infusion, with the differing silencing levels clearly distinguishable. This varied level of detectable HLA-I silencing was sustained through the full 32 days of the experiment, and in both blood and other sampled tissues. In summary, sustained and “tuned” silencing of TCR/CD3 and HLA-I has been demonstrated.

### Methods

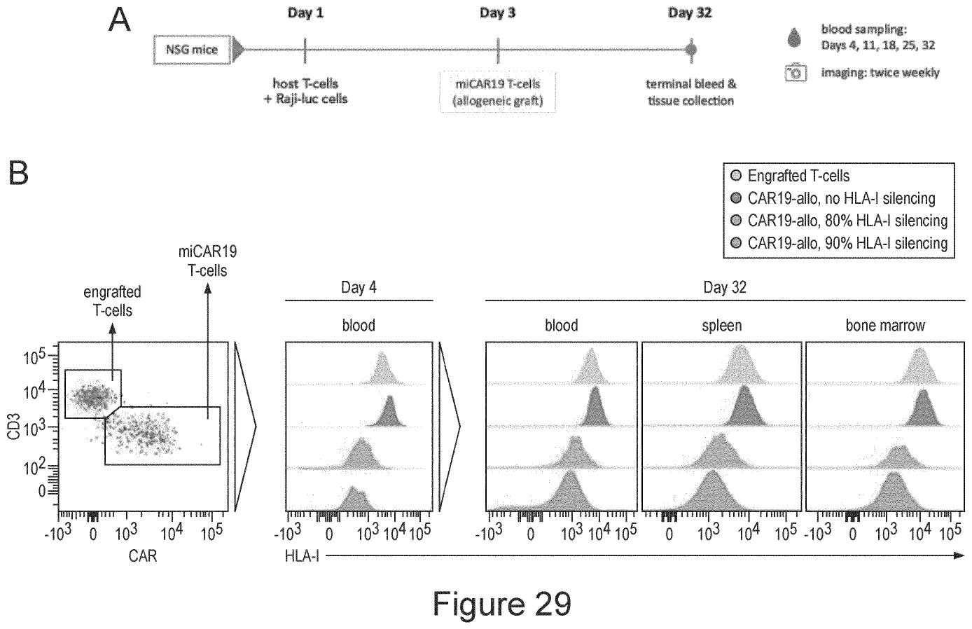

NOD SCID gamma (NSG) mice were infused with “tuned” HLA-I and TCR/CD3-silenced T-cells to assess if tuned HLA-I silencing persists in vivo. FIG. 29A illustrates the study design, where NSG mice were irradiated with a 1 Gy dose, infused with host T-cells and Raji-luc cells 24 hours later, infused with miCAR19 T-cells (allogenic graft) at 3 days, and blood sampled at days 4, 11, 18, 25 and finally 32 where tissue collection was also carried out.

### Results

As shown in FIG. 29, on day 4 (one day post CAR T-cell infusion), blood was collected for flow cytometric analysis to confirm expression of HLA-I. As indicated, “tuned” silencing of HLA-I was clearly detectable across the varying levels. This expression was sustained through to the end of blood sampling at day 32. Additionally, the expression levels of HLA-I at day 32 in the blood were mirrored in the spleen and bone marrow tissue samples also collected at the end of the experiment. FIG. 29B provides the representative histograms indicating the sustained expression of HLA-I based on the day 4 and 32 sampling.

1. An engineered donor cell with reduced rejection by the immune system of a host, wherein one or more cell surface-expressed polypeptides involved in immune signalling are functionally modulated.

2. The engineered donor cell of claim 1, wherein one or more cell surface-expressed polypeptides involved in immune signalling are functionally down-regulated.

3. The engineered donor cell of claim 2, wherein one or more of the down-regulated surface-expressed polypeptides are selected from the group consisting of HLA class I polypeptides and HLA class II polypeptides.

4. The engineered donor cell of claim 3, wherein the down-regulation of HLA class I polypeptides is achieved by miRNA inhibiting the expression of one or more of B2M, NLRC5, TAP1, TAP2, TAPBP, RFX5, RFXANK, and/or RFXAP.

5. The engineered donor cell of claim 3 or 4, wherein the down-regulation of HLA class II polypeptides is achieved by miRNA inhibiting the expression of one or more of CIITA, RFX5, RFXANK, and/or RFXAP.

6. The engineered donor cell of any one of claims 2 to 5, wherein one or more of the down-regulated surface-expressed polypeptides are T-cell receptor (TCR) polypeptides.

7. The engineered donor cell of claim 6, wherein the down-regulation of TCR polypeptides is achieved by miRNA inhibiting the expression of TCRa, TCRb, CD3d, CD3g, CD3e and/or CD3z.

8. The engineered donor cell of any one of claims 2 to 7, wherein one or more of the down-regulated surface-expressed polypeptides are a CD58 polypeptide, optionally wherein the down-regulation is achieved by miRNA targeting the expression of CD58.

9. The engineered donor cell of any one of claims 1 to 8, wherein one or more cell surface-expressed polypeptides involved in immune signalling are up-regulated.

10. The engineered donor cell of claim 9, wherein up-regulation is achieved by an expressed transcript.

11. The engineered donor cell of claim 9 or 10, wherein the up-regulated surface-expressed polypeptide involved in immune signalling is selected from the group consisting of a non-classical HLA class I polypeptide, CD47, PD-L1, and a chimeric antigen receptor (CAR).

12. The engineered donor cell of any one of claims 9 to 11, wherein a surface expressed non-classical HLA class I is up-regulated and a surface-expressed HLA class I is down-regulated.

13. The engineered donor cell of claim 11 or 12, wherein the non-classical HLA class I polypeptide is a genetically modified HLA-E, HLA-G, or HLA-F polypeptide.

14. The engineered donor cell of any one of claims 9 to 13, wherein a surface expressed chimeric antigen receptor (CAR) is up-regulated and a surface expressed T-cell receptor (TCR) is down-regulated.

15. The engineered donor cell of any one of claims 1 to 14, further expressing a safety switch gene or suicide gene.

16. A miRNA expression construct comprising one or more miRNA hairpins targeting B2M, NLRC5, RFX5, RFXANK, RFXAP, CIITA, TCRa, TCRb, CD3d, CD3g, CD3e and/or CD3z; optionally wherein the construct further comprises an expressed transcript.

17. The miRNA expression construct of claim 16, comprising at least a first and a second miRNA hairpin, wherein the first miRNA hairpin and the second miRNA hairpin target a combination of two sequences independently selected from sequences having at least 80%, 85%, 90%, 95%, 96%, 97%, 98%, 99% or 100% to any one of SEQ ID NOs: 1 to 16.

18. The miRNA expression construct of claim 16 or claim 17, wherein the expressed transcript comprises a sequence having at least 80%, 85%, 90%, 95%, 96%, 97%, 98%, 99% or 100% sequence identity to any one of SEQ ID NO: 17, 19 or 21, and/or wherein the expressed transcript encodes a protein having at least 80%, 85%, 90%, 95%, 96%, 97%, 98%, 99% or 100% sequence identity to any one of SEQ ID NO: 18, 20 or 22.

19. The miRNA expression construct of any one of claims 17 to 18, wherein there are two copies of the first miRNA hairpin and/or two copies of the second miRNA hairpin.

20. The miRNA expression construct of any one of claims 17 to 19, wherein there are three copies of the first miRNA hairpin and/or three copies of the second miRNA hairpin.

21. The miRNA expression construct of any one of claims 16 to 20, wherein the construct comprises at least two different miRNA hairpins which target different regions of the same transcript.

22. The miRNA expression construct of any one of claims 16 to 21, wherein the construct comprises at least two different miRNA hairpins which target different transcripts of the same gene.

23. The miRNA expression construct of any one of claims 16 to 22, wherein the construct comprises at least two different miRNA hairpins which target different splice variants of the same gene.

24. The miRNA expression construct of any one of claims 16 to 23 further comprising a promoter element.

25. The miRNA expression construct of claim 24, wherein the promoter element is a promoter.

26. The miRNA expression construct of claim 25, wherein the promoter is a eukaryotic promoter.

27. The miRNA expression construct of claim 26, wherein the eukaryotic promoter is a Pol II or Pol III promoter.

28. The miRNA expression construct of claim 25, wherein the promoter is an inducible promoter, a tissue-specific promoter, a cell lineage-specific promoter or a synthetic promoter.

29. The miRNA expression construct of claim 24, wherein the promoter element is selected from the promoter elements of Table 2.

30. The miRNA expression construct of claim 25, wherein the promoter is a UBI promoter.

31. The miRNA expression construct of claim 25, wherein the promoter is an EF1α promoter, a derivative of an EF1α promoter or an EF1 short promoter.

32. The miRNA expression construct of any one of claims 16 to 24, further comprising a spacer, optionally wherein the spacer comprises an enhancer, further optionally wherein the spacer is an enhancer.

33. The miRNA expression construct of claim 32, wherein the spacer is at least 50 nucleotides in length.

34. The miRNA expression construct of claim 32, wherein the spacer is between 50 and 1,000 nucleotides in length.

35. The miRNA expression construct of claim 32, wherein the spacer is between 50 and 900, 50 and 800, 100 and 800, or 50 and 800 nucleotides in length.

36. The miRNA expression construct of claim 32, wherein the spacer is at least 60, 70, 80, 90, 100, 110, 120, 130, 140, 150, 160, 170, 180 or 200 nucleotides in length.

37. The miRNA expression construct of claim 32, wherein the spacer is a GFP sequence.

38. The miRNA expression construct of any one of claims 32 to 37, wherein the spacer is positioned between the promoter and the miRNA hairpins.

39. The miRNA expression construct of any one of claims 32 to 38, wherein the spacer is heterologous with respect to the promoter element.

40. The miRNA expression construct of any one of claims 32 to 39, wherein the spacer comprises an encoded open reading frame.

41. The miRNA expression construct of any one of claims 16 to 40, wherein at least two of the miRNA hairpins are separated by an intervening sequence.

42. The miRNA expression construct of any one of claims 16 to 41, wherein the expressed transcript is at least one gene selected from the group consisting of non-classical HLA class I, CD47, PD-L1 and a chimeric antigen receptor.

43. The miRNA expression construct of claim 42, wherein the expressed transcript has at least 80%, 85%, 90%, 95% or 100% sequence identity to a sequence set forth in Table 1.

44. The miRNA expression construct of any one of claims 16 to 43, wherein the construct comprises a sequence encoding a moiety for re-directing immune effector cell function.

45. The miRNA expression construct of any one of claims 16 to 44, wherein the construct comprises a sequence encoding an engineered T-cell receptor.

46. The miRNA expression construct of any one of claims 16 to 45, wherein the construct comprises a sequence encoding a chimeric antigen receptor.

47. The miRNA expression construct of claim 45 or claim 46, wherein the miRNA hairpins are under the control of a first promoter and the sequence encoding the T-cell receptor or chimeric antigen receptor is under the control of a second promoter, or wherein the miRNA hairpins and the sequence encoding the T-cell receptor or chimeric antigen receptor are under the control of a single promoter.

48. The miRNA expression construct of any one of claims 16 to 47, wherein the construct further comprises a T cell receptor sequence.

49. The miRNA expression construct of any one of claims 16 to 48, wherein the construct further comprises a selection gene.

50. The miRNA expression construct of claim 49, wherein the selection gene is LNGFR, truncated endothelial growth factor receptor (tEGFR), tCD19, CD20 or a truncated CD20 (tCD20), tCD34 or a derivative thereof.

51. The miRNA expression construct of any one of claims 16 to 50, wherein the construct further comprises a sequence encoding a suicide gene or safety switch gene.

52. The miRNA expression construct of claim 51, wherein the suicide gene or safety switch gene is selected from the group consisting of herpes simplex virus thymidine kinase (HSV-tk), inducible caspase 9 (iCasp9), truncated endothelial growth factor receptor (EGFR), RQR8, dihydrofolate reductase (DHFR), CD20 or a truncated CD20 (tCD20) and thymidylate synthase (TYMS).

53. The miRNA expression construct of any one of claims 16 to 52, wherein the construct further comprises an internal ribosome entry site (IRES).

54. A DNA molecule comprising the miRNA expression construct of any one of claims 16 to 53.

55. A plasmid comprising the miRNA expression construct or DNA molecule of any one of claims 16 to 54.

56. A vector comprising the miRNA expression construct, DNA molecule or plasmid of any one of claims 16 to 55.

57. The vector of claim 56, wherein the vector is an expression vector.

58. The expression vector of claim 57, wherein the expression vector is an adenovirus, an adeno-associated virus, a retrovirus or a lentivirus vector.

59. The expression vector of claim 57 or claim 58, further comprising at least one drug resistance marker.

60. An engineered donor cell comprising the miRNA expression construct, DNA molecule, plasmid or vector of any one of claims 16 to 59.

61. A method for down-regulating a polypeptide in a cell comprising expressing the miRNA expression construct, DNA molecule, plasmid or vector of any one of claims 16 to 59 in the cell.

62. A method for preparing an engineered donor cell comprising transfecting or transducing a cell with the miRNA expression construct, DNA molecule, plasmid or vector of any one of claims 16 to 59.

63. A method for preparing an engineered donor cell from a patient donor or healthy donor comprising:
(a) collecting a cell from the patient; and
(b) transfecting or transducing the cell with the miRNA expression construct, DNA molecule, plasmid or vector of any one of claims 16 to 59; and
(c) expressing the miRNA expression construct.

64. The method of claim 62 or 63, wherein the engineered donor cell is a T-cell.

65. The method of any one of claims 62 to 64, wherein the miRNA expression construct, DNA molecule, plasmid or vector down-regulates a TCR polypeptide and up-regulates a CAR polypeptide, wherein the engineered donor cell is a CAR T-cell.

66. The method of claim 65, wherein the chimeric antigen receptor targets HIV infected cells or tumour cells, optionally wherein the chimeric antigen receptor is an anti-CD19 chimeric antigen receptor, optionally wherein the chimeric antigen receptor is FMC63.

67. An engineered donor cell obtainable or obtained by the method of any one of claims 62 to 66.

68. The engineered donor cell of any one of claim 1 to 15, 60 or 67, wherein the engineered donor cell is a eukaryotic cell, optionally wherein the engineered donor cell is a mammalian cell.

69. The engineered donor cell of any one of claim 1 to 15, 60, 67 or 68, wherein the engineered donor cell in an immune effector cell.

70. The engineered donor cell of claim 69, wherein the immune effector cell is selected from the group comprising: alpha-beta T-cells, gamma-delta T-cells, tumour infiltrating lymphocytes (TILS), TCR-engineered T-cells, CAR T-cells, NK cells, NK/T-cells, T regulatory cells, monocytes and macrophages.

71. The engineered donor cell of claim 70, wherein the immune effector cell is a CAR T-cell.

72. The engineered donor cell of any one of claim 1 to 15, 60, 67 or 68, wherein the engineered donor cell is a stem cell or a progenitor cell.

73. The engineered donor cell of claim 72, wherein the engineered donor cell is a pluripotent stem cell, such as an embryonic and/or an induced pluripotent stem cell.

74. The engineered donor cell of claim 72, wherein the engineered donor cell is a multipotent stem cell, such as a haematopoietic stem cell, mesenchymal stem cell, neural stem cell or a muscle stem cell (satellite cell).

75. The engineered donor cell of any one of claim 1 to 15, 60, 67 or 68, wherein the engineered donor cell is a differentiated cell.

76. The engineered donor cell of claim 75, wherein the engineered donor cell is a pancreatic cell, optionally a pancreatic islet cell or pancreatic β cell.

77. A composition comprising the engineered donor cell of any one of claims 1 to 15, 60 or 67 to 76.

78. The engineered donor cell, miRNA expression construct, DNA molecule, plasmid, vector or composition of any one of claims 1 to 60 or 67 to 77, for use in therapy.

79. The engineered donor cell, miRNA expression construct, DNA molecule, plasmid, vector or composition of any one of claims 1 to 60 or 67 to 77, for use in a method of treating cancer, an infectious disease, an auto-immune disease or an inherited disorder.

80. A method of treating cancer, an infectious disease, an auto-immune disease or an inherited disorder, comprising administering the engineered donor cell, miRNA expression construct, DNA molecule, plasmid, vector or composition of any one of claims 1 to 60 or 67 to 77.

81. The engineered donor cell, miRNA expression construct, DNA molecule, plasmid, vector or composition of any one of claims 1 to 60 or 67 to 77, for use in the manufacture a medicament for the treatment of cancer, an infectious disease, an auto-immune disease or an inherited disorder.

82. The engineered donor cell, miRNA expression construct, DNA molecule, plasmid, vector or composition of any one of claims 1 to 60 or 67 to 77, for use in a method of stem cell therapy.

83. A method of stem cell therapy, comprising administering the engineered donor cell, miRNA expression construct, DNA molecule, plasmid, vector or composition of any one of claims 1 to 60 or 67 to 77.

84. The engineered donor cell, miRNA expression construct, DNA molecule, plasmid, vector or composition of any one of claims 1 to 60 or 67 to 77, for use in the manufacture a medicament for stem cell therapy.


SEQUENCE LISTING (57 sequences)

SEQ ID NO: 1 - DNA, 22 bp, synthetic construct
  source 1..22: mol_type = other DNA
ttgactttccattctctgctgg

SEQ ID NO: 2 - DNA, 22 bp, synthetic construct
  source 1..22: mol_type = other DNA
ttatgcacgcttaactatctta

SEQ ID NO: 3 - DNA, 22 bp, synthetic construct
  source 1..22: mol_type = other DNA
taaacctgaatctttggagtac

SEQ ID NO: 4 - DNA, 22 bp, synthetic construct
  source 1..22: mol_type = other DNA
tttgcatagaagataaccttcc

SEQ ID NO: 5 - DNA, 22 bp, synthetic construct
  source 1..22: mol_type = other DNA
ttagtctgtgagtaagcaaggc

SEQ ID NO: 6 - DNA, 22 bp, synthetic construct
  source 1..22: mol_type = other DNA
tatagaccaacaatcatgtatc

SEQ ID NO: 7 - DNA, 22 bp, synthetic construct
  source 1..22: mol_type = other DNA
tgaaagcatagcctgtctgctg

SEQ ID NO: 8 - DNA, 22 bp, synthetic construct
  source 1..22: mol_type = other DNA
tttccaaggacttcagctgggg

SEQ ID NO: 9 - DNA, 22 bp, synthetic construct
  source 1..22: mol_type = other DNA
ttagtgtcctcagagaacatgc

SEQ ID NO: 10 - DNA, 22 bp, synthetic construct
  source 1..22: mol_type = other DNA
tattgtacaagcttagcctgag

SEQ ID NO: 11 - DNA, 22 bp, synthetic construct
  source 1..22: mol_type = other DNA
tttggagctaaatataaccaaa

SEQ ID NO: 12 - DNA, 22 bp, synthetic construct
  source 1..22: mol_type = other DNA
tatcctagtacattgacgggtt

SEQ ID NO: 13 - DNA, 22 bp, synthetic construct
  source 1..22: mol_type = other DNA
ttccacttcatcttgtcctttc

SEQ ID NO: 14 - DNA, 22 bp, synthetic construct
  source 1..22: mol_type = other DNA
tcatgagcagattaaacccggc

SEQ ID NO: 15 - DNA, 22 bp, synthetic construct
  source 1..22: mol_type = other DNA
ttaggttcgtatctgtttcaaa

SEQ ID NO: 16 - DNA, 22 bp, synthetic construct
  source 1..22: mol_type = other DNA
tcagatttgttgctccaggcca

SEQ ID NO: 17 - DNA, 2025 bp, synthetic construct
  source 1..2025: mol_type = other DNA
atgcttctcctggtgacaagccttctgctctgtgagttaccacacccagcattcctcctgatcccagcatgcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcacgtacgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtatgcatggttccgcagccgctgagggcagaggaagtcttctaacatgcggtgacgtggaggagaatcccggccctttcgaaatgctgaccagcctgctgtgctggatggccctgtgcctgctgggcgccgaccacgccgatgcctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtagcggagggggaggcagcccagcccccagacctcccaccccagcccccaccatcgccagccagcctctgagcctgagacccgaggcctgccgcccagccgccggcggcgccgtgcacaccagaggcctggatttcgcctgcgatatctacatctgggccccactggccggcacctgtggcgtgctgctgctgagcctggtgatcaccctgtactgcaaccaccgcaaccgcaggcgcgtgtgcaagtgccccaggcccgtggtgtga

SEQ ID NO: 18 - protein, 674 aa, synthetic construct
MLLLVTSLLLCELPHPAFLLIPACDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRTKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMHGSAAAEGRGSLLTCGDVEENPGPFEMLTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 19 - DNA, 1500 bp, synthetic construct
  source 1..1500: mol_type = other DNA
atgtctcgctccgtggccttagctgtgctcgcgctactctctctttctggcctcgaggctgttatggctccgcggactttaattttaggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttg

SEQ ID NO: 20 - protein, 500 aa, synthetic construct
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 21 - DNA, 1500 bp, synthetic construct
  source 1..1500: mol_type = other DNA
atgtctcgctccgtggccttagctgtgctcgcgctactctctctttctggcctcgaggctgttatggctccgcggactttaattttaggtggtggcggatccggtggtggcggttctggtggtggcggctccatccagcgtacgccgaagatacaggtttactcacgtcacccagccgaaaatggcaagtctaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcacggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttg

SEQ ID NO: 22 - protein, 500 aa, synthetic construct; identical to SEQ ID NO: 20

SEQ ID NO: 23 - DNA, 876 bp, synthetic construct
  source 1..876: mol_type = other DNA
atgtggcctctggttgctgctctgctgctgggctctgcctgttgtggatctgcccagctgctgttcaacaagaccaagagcgtcgagttcaccttctgcaacgataccgtggtcatcccctgcttcgtgaccaacatggaagcccagaacaccaccgaggtgtacgtgaagtggaagttcaagggcagagacatctacaccttcgacggcgccctgaacaagagcaccgtgcctacagatttcagcagcgccaagatcgaggtgtcacagctgcttaagggcgacgccagcctgaagatggataagagcgacgccgtgtctcacaccggcaactacacctgtgaagtgaccgagctgaccagagagggcgagacaatcatcgagctgaagtacagagtggtgtcctggttcagccccaacgagaacatcctgatcgtgatcttccccatcttcgccatcctgctgttctggggccagttcggcatcaagaccctgaagtatagaagcggcggcatggacgagaaaacaatcgccctgctggtggccggcctggtcatcacagtgattgtgatcgtgggagccatcctgttcgtgcccggcgagtactctctgaagaatgccacaggcctgggcctgattgtgaccagcacaggcatcctgattctgctgcactactacgtgttcagcaccgccatcggcctgaccagctttgtgatcgccattctggttatccaagtgatcgcctacatcctggccgtcgtgggcctgtctctgtgtatcgccgcctgcattcctatgcacggccctctgctgatctccggcctgtctattctggctctggctcagctgctcggcctggtgtatatgaagttcgtg

SEQ ID NO: 24 - protein, 292 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 25 - DNA, 870 bp, synthetic construct
  source 1..870: mol_type = other DNA
atgagaatcttcgccgtgttcatctttatgacctactggcatctgctgaacgccttcaccgtgaccgtgcctaaggacctgtacgtggtggaatacggcagcaacatgaccatcgagtgcaagttccccgtggaaaagcagctggatctggccgctctgatcgtgtactgggagatggaagataagaacatcatccagttcgtgcatggcgaagaggacctgaaggtgcagcacagcagctacagacagagagccagactgctgaaggaccagctgagcctgggaaatgccgctctccagatcaccgacgtgaagctgcaagatgccggcgtgtacaggtgcatgatctcttatggcggagccgactacaagcggatcaccgtgaaagtgaacgccccttacaacaagatcaaccagagaatcctggtggtggaccccgtgacatctgagcacgagctgacttgtcaggccgagggctatcctaaggccgaagtgatctggaccagcagcgatcaccaggtgctgagcggcaagaccaccaccacaaacagcaagcgggaagagaagctgttcaacgtgaccagcacactgcggatcaacacaaccaccaacgagatcttctactgcacctttcggcggctggaccccgaggaaaatcacacagccgagctggtcatccccgagctgcctcttgctcaccctcctaacgagagaacccacctcgtgatcctgggagccatcctgctgtgtctgggagtcgccctgaccttcatcttccggctgagaaagggcagaatgatggacgtgaagaagtgcggcatccaggacaccaacagcaagaagcagagcgacacccacctggaagagaca

SEQ ID NO: 26 - protein, 290 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 27 - DNA, 418 bp, synthetic construct
  source 1..418: mol_type = other DNA
atgtctcgctccgtggccttagctgtgctcgcgctactctctctttctggcctcgaggctgttatggctccgcggactttaattttaggtggtggcggatccggtggtggcggttctggtggtggcggctccatccagcgtacgccgaagatacaggtttactcacgtcacccagccgaaaatggcaagtctaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggg

SEQ ID NO: 28 - DNA, 375 bp, synthetic construct
  source 1..375: mol_type = other DNA
aattcatggatgtaagtaggtgagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacc

SEQ ID NO: 29 - DNA, 394 bp, synthetic construct
  source 1..394: mol_type = other DNA
aattccaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggcctgaatcgccagtgtc

SEQ ID NO: 30 - DNA, 22 bp, synthetic construct
  source 1..22: mol_type = other DNA
ccagcagagaatggaaagtcaa

SEQ ID NO: 31 - DNA, 22 bp, synthetic construct
  source 1..22: mol_type = other DNA
gtactccaaagattcaggttta

SEQ ID NO: 32 - DNA, 22 bp, synthetic construct
  source 1..22: mol_type = other DNA
cgtgcataagttaacttccaat

SEQ ID NO: 33 - DNA, 22 bp, synthetic construct
  source 1..22: mol_type = other DNA
gctgtctccatgtttgatgtat

SEQ ID NO: 34 - DNA, 22 bp, synthetic construct
  source 1..22: mol_type = other DNA
ggttgtggttaatctggtttat

SEQ ID NO: 35 - DNA, 22 bp, synthetic construct
  source 1..22: mol_type = other DNA
ctctgcttagaatttgggggaa

SEQ ID NO: 36 - DNA, 22 bp, synthetic construct
  source 1..22: mol_type = other DNA
catccgacattgaagttgactt

SEQ ID NO: 37 - DNA, 22 bp, synthetic construct
  source 1..22: mol_type = other DNA
ccagcgtactccaaagattcag

SEQ ID NO: 38 - DNA, 22 bp, synthetic construct
  source 1..22: mol_type = other DNA
ccccactgaaaaagatgagtat

SEQ ID NO: 39 - DNA, 22 bp, synthetic construct
  source 1..22: mol_type = other DNA
cgtactccaaagattcaggttt

SEQ ID NO: 40 - DNA, 22 bp, synthetic construct
  source 1..22: mol_type = other DNA
aaggcatggttgtggttaatct

SEQ ID NO: 41 - DNA, 22 bp, synthetic construct
  source 1..22: mol_type = other DNA
gactggtctttctatctcttgt

SEQ ID NO: 42 - DNA, 22 bp, synthetic construct
  source 1..22: mol_type = other DNA
gatcgagacatgtaagcagcat

SEQ ID NO: 43 - DNA, 22 bp, synthetic construct
  source 1..22: mol_type = other DNA
ttgctatgtgtctgggtttcat

SEQ ID NO: 44 - DNA, 22 bp, synthetic construct
  source 1..22: mol_type = other DNA
taagatagttaagcgtgcataa

SEQ ID NO: 45 - DNA, 22 bp, synthetic construct
  source 1..22: mol_type = other DNA
ggaaggttatcttctatgcaaa

SEQ ID NO: 46 - DNA, 22 bp, synthetic construct
  source 1..22: mol_type = other DNA
gccttgcttactcacagactaa

SEQ ID NO: 47 - DNA, 22 bp, synthetic construct
  source 1..22: mol_type = other DNA
gatacatgattgttggtctata

SEQ ID NO: 48 - DNA, 22 bp, synthetic construct
  source 1..22: mol_type = other DNA
cagcagacaggctatgctttca

SEQ ID NO: 49 - DNA, 22 bp, synthetic construct
  source 1..22: mol_type = other DNA
ccccagctgaagtccttggaaa

SEQ ID NO: 50 - DNA, 22 bp, synthetic construct
  source 1..22: mol_type = other DNA
gcatgttctctgaggacactaa

SEQ ID NO: 51 - DNA, 22 bp, synthetic construct
  source 1..22: mol_type = other DNA
ctcaggctaagcttgtacaata

SEQ ID NO: 52 - DNA, 22 bp, synthetic construct
  source 1..22: mol_type = other DNA
tttggttatatttagctccaaa

SEQ ID NO: 53 - DNA, 22 bp, synthetic construct
  source 1..22: mol_type = other DNA
aacccgtcaatgtactaggata

SEQ ID NO: 54 - DNA, 22 bp, synthetic construct
  source 1..22: mol_type = other DNA
gaaaggacaagatgaagtggaa

SEQ ID NO: 55 - DNA, 22 bp, synthetic construct
  source 1..22: mol_type = other DNA
gccgggtttaatctgctcatga

SEQ ID NO: 56 - DNA, 22 bp, synthetic construct
  source 1..22: mol_type = other DNA
tttgaaacagatacgaacctaa

SEQ ID NO: 57 - DNA, 22 bp, synthetic construct
  source 1..22: mol_type = other DNA
tggcctggagcaacaaatctga